SEQUENCE LISTING (27 sequences)

SEQ ID NO: 1 - DNA, 192 bp, Artificial Sequence
  Synthetic
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 2 - DNA, 3000 bp, Artificial Sequence
  Synthetic
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 3 - RNA, 6 nt, Artificial Sequence
  Synthetic
  repeat_unit (1)..(6): may repeat up to 100,000 times or more
ggggcc

SEQ ID NO: 4 - protein, 4 aa, Artificial Sequence
  Synthetic
LPAC

SEQ ID NO: 5 - protein, 4 aa, Artificial Sequence
  Synthetic
QAGR

SEQ ID NO: 6 - protein, 8 aa, Artificial Sequence
  Synthetic
FTPLSLPV

SEQ ID NO: 7 - protein, 8 aa, Artificial Sequence
  Synthetic
LLPSPSRC

SEQ ID NO: 8 - protein, 8 aa, Artificial Sequence
  Synthetic
YSPLPPGV

SEQ ID NO: 9 - protein, 8 aa, Artificial Sequence
  Synthetic
HREGEGSK

SEQ ID NO: 10 - protein, 8 aa, Artificial Sequence
  Synthetic
TGRERGVN

SEQ ID NO: 11 - protein, 6 aa, Artificial Sequence
  Synthetic
PGGRGE

SEQ ID NO: 12 - protein, 8 aa, Artificial Sequence
  Synthetic
GRQRGVNT

SEQ ID NO: 13 - protein, 8 aa, Artificial Sequence
  Synthetic
GSKHREAE

SEQ ID NO: 14 - DNA, 20 bp, Artificial Sequence
  Synthetic
ggcaccatgtctggtttgtt

SEQ ID NO: 15 - DNA, 20 bp, Artificial Sequence
  Synthetic
agactggtcggtgatctcgt

SEQ ID NO: 16 - DNA, 18 bp, Artificial Sequence
  Synthetic
agaacaggacaagttgcc

SEQ ID NO: 17 - DNA, 19 bp, Artificial Sequence
  Synthetic
aacacacacctcctaaacc

SEQ ID NO: 18 - DNA, 21 bp, Artificial Sequence
  Synthetic
agttgggtccatgctcaacaa

SEQ ID NO: 19 - DNA, 20 bp, Artificial Sequence
  Synthetic
actgttctaggtaccgggct

SEQ ID NO: 20 - DNA, 20 bp, Artificial Sequence
  Synthetic
gcccacgtaaaagatgacgc

SEQ ID NO: 21 - DNA, 20 bp, Artificial Sequence
  Synthetic
cctcctaaacccacacctgc

SEQ ID NO: 22 - DNA, 19 bp, Artificial Sequence
  Synthetic
agtcgctagaggcgaaagc

SEQ ID NO: 23 - DNA, 19 bp, Artificial Sequence
  Synthetic
cgagtgggtgagtgaggag

SEQ ID NO: 24 - DNA, 20 bp, Artificial Sequence
  Synthetic
tctccagctgttgccaagac

SEQ ID NO: 25 - DNA, 21 bp, Artificial Sequence
  Synthetic
tccattctctctgtgccttct

SEQ ID NO: 26 - DNA, 20 bp, Artificial Sequence
  Synthetic
tcgtgcgtgacatcaaagag

SEQ ID NO: 27 - DNA, 20 bp, Artificial Sequence
  Synthetic
gatcttcatggtgctaggag

(19) United States
(12) Patent Application Publication
(10) Pub. No.: US 2023/0304088 A1
(43) Pub. Date: Sep. 28, 2023

(54) COMPOSITIONS AND METHODS OF DETECTION OF PRE-SYMPTOMATIC ALS

(71) Applicant: University of Florida Research Foundation, Incorporated, Gainesville, FL (US)

(72) Inventors: Laura Ranum, Gainesville, FL (US); Amrutha Pattamatta, Gainesville, FL (US); Eric Tzy-Shi Wang, Gainesville, FL (US); Hailey Olafson, Gainesville, FL (US)

(73) Assignee: University of Florida Research Foundation, Incorporated, Gainesville, FL (US)

(21) Appl. No.: 17/925,054

(22) PCT Filed: May 14, 2021
(86) PCT No.: PCT/US2021/032488

§ 371 Date: Nov. 14, 2022

Related U.S. Application Data
(60) Provisional application No. 63/025,446, filed on May 15, 2020.

(51) Int. Cl. C12Q 1/6883; G01N 33/68

(52) U.S. Cl. CPC C12Q 1/6883; G01N 33/6875; C12Q 2600/118; C12Q 2600/156; C12Q 2600/158

(57) ABSTRACT

Aspects of the disclosure relate to methods and compositions (e.g., biomarkers) useful for diagnosing pre- symptomatic amyotrophic lateral sclerosis (ALS) and/or frontotemporal dementia (FTD), and for monitoring the progression of ALS/FTD in subjects diagnosed with the disease. Methods of treating neurodegenerative diseases (e.g., ALS/FTD) are also described.

### RELATED APPLICATION

This application claims the benefit under 35 U.S.C. § 119(e) of the filing date of U.S. Provisional Application Serial No. 63/025,446, filed May 15, 2020 and entitled “COMPOSITIONS AND METHODS OF DETECTION OF PRE-SYMPTOMATIC ALS”, the entire contents of which are incorporated herein by reference.

### FEDERALLY SPONSORED RESEARCH

This invention was made with government support under grant number R01 NS098819, awarded by the National Institutes of Health. The government has certain rights in the invention.

### STATEMENT OF NON-FEDERAL FUNDING

This invention was made in whole or in part from funding received under contract number AGR00012010, received from the Amyotrophic Lateral Sclerosis Association.

### BACKGROUND

Expansion of a GGGGCC hexanucleotide sequence within the intron of the human C9orf72 gene is the most commonly known genetic cause of amyotrophic lateral sclerosis (ALS) and frontotemporal dementia (FTD) in humans. However, ascertainment bias, technical difficulties in measuring repeat length, and somatic instability have made understanding the effects of repeat length as a modifier of C9orf72 ALS/FTD difficult.

### SUMMARY

In some aspects, the disclosure relates to methods for the diagnosis and treatment of amyotrophic lateral sclerosis (ALS) and frontotemporal dementia (FTD). The disclosure is based, in part, on the discovery that certain isoforms of certain genes as described herein undergo alternative splicing changes that are detectable in a biological sample (e.g., blood, serum, or tissue) of a subject having or at risk of having pre-symptomatic ALS/FTD.

Aspects of the disclosure relate to detection (e.g., quantification) of an increase or decrease (e.g., a psi (Ψ) score) in the level of certain isoforms of certain genes as described herein that are known to be altered in C9-positive acute ALS/FTD in subjects with C9-negative ALS/FTD or pre-symptomatic C9-positive ALS/FTD can be used as an early biomarker for the disease.

Accordingly, in some aspects, the disclosure provides for a method for identifying a subject as having pre-symptomatic amyotrophic lateral sclerosis (ALS), the method comprising:
(i) detecting (e.g., quantifying) levels of two or more isoforms of one or more genes selected from: Nek6, Pphln1, Pdgfc, Pomt1, Sorbs1, Ssfa2, Rps6kb2, Irak1, Cpeb4, Calu, Gtpbp10, Pard3, Ranbp3, Prx, Rad18, 2810474O19Rik, Mtdh, Sema6d, Arl6, Tbp, Slx4, Abi1, Osbpl1a, Pex7, Camklg, Idnk, Zfp963, Firre, Grik1, Ktn1, Nmbr, Sec14l2, Gm21992, Mpnd, Elavl2, Mtmr1, Six3os1, Tenm4, Zyx, Camsap3, Brat1, Hdac5, Fmnl3, Pisd, Atp11c, Lair1, Ccnc, Nnat, Fam111a, Mettl4, D130020L05Rik, Etfrf1, Chd2, Ikzf4, Phkb, A730017C20Rik, Tnrc6a, Itga3, Gprasp1, Ptprf, Cd200, Dync1i2, Huwe1, Aplp2, Ctbs, Mink1, Dpy19l4, Ccdc85a, Asph, Nr3c2, Kif2a, Dennd5a, Pip5k1c, Plxna3, Arhgef9, Iqsec2, Fyttd1, Smyd4, Tmem234, Aif1l, Adgrl3, 0610037L13Rik, 1600014C10Rik, 1700001L05Rik, 3010001F23Rik, 6430573F11Rik, 9030617003Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcrl, Camkk2, Ccdc43, Ccdc66, Cd180, Cdk14, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Dcaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4g1, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Galns, Glt8d1, Gm20319, Gm28042, Gng12, Golgb1, Gphn, Gpm6b, Gprin1, Gramd3, Gsn, H13, H2-Q7, H2-T22, Histh2bq, Hist1h2br, Hnrnpa2b1, Hrasls, Hrhl, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcntl, Klc4, Las1l, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf4l2, Morn1, Mpv17l2, Mthfsl, Myole, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pcdh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfial, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassf2, Rbm10, Rbm12, Rbm39, Rbms3, Rcbtb2, Retreg3, Rffl, Rhobtb1, Rhoj, Ripor2, Rpain, Rpgr, Rps24, Rps6kal, Rtell, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1, Snap23, Snrpa1, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd52l2, Trp53i13, Trpc1, Ttc17, Ttc21b, Ttll5, Tysnd1, Ube2cbp, Ubn2, Ubxn7, Uhrf1bp1, Unc13b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Spp1, Plek, Col5a3, Slpr3, Anxa2, Mt1, Sbno2, Csf1, Ch25h, Mt2, Serpine1, Vim, Col16a1, Neat1, Cd109, Dysf, C3ar1, Pros1, Tnfrsf1a, Tgm1, Socs3, Rplp0, Osmr, Cebpd, Tspan7, Srxn1, Capg, Ahnak, Fgf13, S100a10, Arpc1b, Slc6a1, Lgals3, Bcl3, Plin2, Csf2rb2, Clic1, Gfap, Aspg, Emp1, and/or Msr1 in a biological sample (e.g., blood, serum, or tissue) obtained from a subject;
(ii) calculating a psi (Ψ) score for each of the one or more genes based on the detecting of (i);
(iii) comparing each Ψ score of (ii) to a Ψ score of the same gene from a control sample to produce a delta psi (ΔΨ) score; and,
(iv) identifying the subject as having pre-symptomatic ALS if the absolute value of the ΔΨ score is not zero (0).

In some aspects, the disclosure provides a method for monitoring disease progression in a subject by calculating the ΔΨ in two or more isoforms of one or more genes, calculated as described herein, in the same subject over a period of time. In some embodiments, this method comprises:
(i) detecting (e.g., quantifying) in a first biological sample obtained from a subject (e.g., a subject that is genopositive for a C9orf72 expansion repeat) a first psi (Ψ) score of one or more genes selected from: Pphln1, Pdgfc, Pomt1, Sorbs1, Ssfa2, Rps6kb2, Irak1, Cpeb4, Calu, Gtpbp10, Pard3, Ranbp3, Prx, Rad18, 2810474O19Rik, Mtdh, Sema6d, Arl6, Tbp, Slx4, Abi1, Osbpl1a, Pex7, Camklg, Idnk, Zfp963, Firre, Grik1, Ktn1, Nmbr, Sec14l2, Gm21992, Mpnd, Elavl2, Mtmr1, Six3os1, Tenm4, Zyx, Camsap3, Brat1, Hdac5, Fmnl3, Pisd, Atp11c, Lair1, Ccnc, Nnat, Fam111a, Mettl4, D130020L05Rik, Etfrf1, Chd2, Ikzf4, Phkb, A730017C20Rik, Tnrc6a, Itga3, Gprasp1, Ptprf, Cd200, Dync1i2, Huwe1, Aplp2, Ctbs, Mink1, Dpy19l4, Ccdc85a, Asph, Nr3c2, Kif2a, Dennd5a, Pip5k1c, Plxna3, Arhgef9, Iqsec2, Fyttd1, Smyd4, Tmem234, Aif1l, Adgrl3, 0610037L13Rik, 1600014C10Rik, 1700001L05Rik, 3010001F23Rik, 6430573F11Rik, 9030617003Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcrl, Camkk2, Ccdc43, Ccdc66, Cd180, Cdk14, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Dcaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4g1, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Galns, Glt8d1, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcnt1, Klc4, Las1l, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf4l2, Morn1, Mpv17l2, Mthfsl, Myo1e, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pcdh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfial, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassf2, Rbm10, Rbm12, Rbm39, Rbms3, Rcbtb2, Retreg3, Rffl, Rhobtb1, Rhoj, Ripor2, Rpain, Rpgr, Rps24, Rps6ka1, Rtel1, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50al, Smoc1, Snap23, Snrpa1, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd52l2, Trp53i13, Trpc1, Ttc17, Ttc21b, Ttll5, Tysnd1, Ube2cbp, Ubn2, Ubxn7, Uhrf1bp1, Unc13b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Spp1, Plek, Col5a3, S1pr3, Anxa2, Mt1, Sbno2, Csf1, Ch25h, Mt2, Serpine1, Vim, Co116a1, Neat1, Cd109, Dysf, C3ar1, Pros1, Tnfrsf1a, Tgm1, Socs3, Rplp0, Osmr, Cebpd, Tspan7, Srxn1, Capg, Ahnak, Fgfl3, S100a10, Arpc1b, Slc6a1, Lgals3, Bcl3, Plin2, Csf2rb2, Clic1, Gfap, Aspg, Emp1, and/or Msr1;
(ii) detecting (e.g., quantifying) in a second biological sample obtained from the subject a second psi (ψ) score of one or more genes selected from: Nek6, Pphln1, Pdgfc, Pomt1, Sorbs1, Ssfa2, Rps6kb2, Irak1, Cpeb4, Calu, Gtpbp10, Pard3, Ranbp3, Prx, Rad18, 2810474019Rik, Mtdh, Sema6d, Arl6, Tbp, Slx4, Abi1, Osbpl1a, Pex7, Camklg, Idnk, Zfp963, Firre, Grik1, Ktn1, Nmbr, Sec1412, Gm21992, Mpnd, Elavl2, Mtmr1, Six3osl, Tenm4, Zyx, Camsap3, Brat1, Hdac5, Fmnl3, Pisd, Atp11c, Lair1, Ccnc, Nnat, Fam111a, Mett14, D130020L05Rik, Etfrf1, Chd2, Ikzf4, Phkb, A730017C20Rik, Tnrc6a, Itga3, Gprasp1, Ptprf, Cd200, Dyncli2, Huwe1, Aplp2, Ctbs, Mink1, Dpy19l4, Ccdc85a, Asph, Nr3c2, Kif2a, Dennd5a, Pip5klc, Plxna3, Arhgef9, Iqsec2, Fyttd1, Smyd4, Tmem234, Aif1l, Adgrl3, 0610037L13Rik, 1600014C10Rik, 1700001L05Rik, 3010001F23Rik, 6430573FIIRik, 9030617003Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgej7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcrl, Camkk2, Ccdc43, Ccdc66, Cd180, Cdk14, Celj2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Deaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4g1, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrs11, Fbxo34, Fcgr2b, Fgfrlop2, Flna, Fn1, Fxr1, Fyn, G3bp2, Galns, Glt8d1, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcnt1, Klc4, Lasl1, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf4l2, Morn1, Mpv1712, Mthfs1, Myole, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pedh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfia1, Ppplr16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassf2, Rbm10, Rbm12, Rbm39, Rbms3, Rcbtb2, Retreg3, Rffl, Rhobtb1, Rhoj, Ripor2, Rpain, Rpgr, Rps24, Rps6kal, Rtel1, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1, Snap23, Snrpa1, Snx21, Spint2, Spire1, Ssr1, St3gal3, St3gal6, Stardl3, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd5212, Trp53il3, Trpc1, Ttc17, Ttc21b, Ttll5, Tysnd1, Ube2cbp, Ubn2, Ubxn7, Uhrf1bp1, Uncl3b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Spp1, Plek, Col5a3, Slpr3, Anxa2, Mt1, Sbno2, CSf1, Ch25h, Mt2, Serpine1, Vim, Col16a1, Neat1, Cd109, Dysf, C3arl, Pros1, Tnfrsf1a, Tgm1, Socs3, RplpO, Osmr, Cebpd, Tspan7, Srxn1, Capg, Ahnak, Fgf13, S100a10, Arpc1b, Slc6a1, Lgals3, Bcl3, Plin2, Csf2rb2, Clic1, Gfap, Aspg, Emp1, and/or Msr1; and
(iii) diagnosing the subject as having progressed from a pre-symptomatic ALS disease state to an acute ALS disease state if the value of the second Ψ score is increased or decreased relative to the first Ψ score.

In further aspects, the disclosure provides a method for treating pre-symptomatic ALS in a subject, the method comprising administering to the subject a therapeutic agent, wherein the subject has been characterized as having pre-symptomatic ALS by the methods described herein.

In some embodiments of methods described by the disclosure, the biological sample is blood, serum, tissue (e.g., tissue from the central nervous system of the subject), or cerebrospinal fluid (CSF). In some embodiments, the levels of two or more isoforms of one or more genes selected from: Nek6, Pphln1, Pdgfc, Pomt1, Sorbs1, Ssfa2, Rps6kb2, Irak1, Cpeb4, Calu, Gtpbp10, Pard3, Ranbp3, Prx, Rad18, 2810474019Rik, Mtdh, Sema6d, Arl6, Tbp, Slx4, Abi1, Osbpl1a, Pex7, Camklg, Idnk, Zfp963, Firre, Grik1, Ktn1, Nmbr, Sec14l2, Gm21992, Mpnd, Elavl2, Mtmr1, Six3osl, Tenm4, Zyx, Camsap3, Brat1, Hdac5, Fmnl3, Pisd, Atp11c, Lair1, Ccnc, Nnat, Fam111a, Mettl4, D130020L05Rik, Etfrf1, Chd2, Ikzf4, Phkb, A730017C20Rik, Tnrc6a, Itga3, Gprasp1, Ptprf, Cd200, Dyncli2, Huwe1, Aplp2, Ctbs, Mink1, Dpy19l4, Ccdc85a, Asph, Nr3c2, Kif2a, Dennd5a, Pip5kle, Plxna3, Arhgej9, Iqsec2, Fyttd1, Smyd4, Tmem234, Aif1l, Adgrl3, 0610037L13Rik, 1600014C10Rik, 1700001L05Rik, 3010001F23Rik, 6430573F11Rik, 9030617003Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4bl, Ap4el, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcr1, Camkk2, Ccdc43, Ccdc66, Cd180, Cdk14, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Dcaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4gl, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Gains, Glt8dl, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcntl, Klc4, Las1l, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf412, Morn1, Mpv17l2, Mthfsl, Myo1e, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pedh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfia1, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassf2, Rbm10, Rbm12, Rbm39, Rbms3, Rcbtb2, Retreg3, Rffl, Rhobtb1, Rhoj, Ripor2, Rpain, Rpgr, Rps24, Rps6ka1, Rtel1, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, S1c31a2, Slc38a10, Slc39a13, Slc39a9, Slc4a7, Slc50a1, Smoc1, Snap23, Snrpa1, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd52l2, Trp53i13, Trpc1, Ttc17, Ttc21b, Ttll5, Tysndl, Ube2cbp, Ubn2, Ubxn7, Uhrf1bp1, Unc13b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Spp1, Plek, Col5a3, S1pr3, Anxa2, Mt1, Sbno2, Csf1, Ch25h, Mt2, Serpine1, Vim, Col16a1, Neat1, Cd109, Dysf, C3ar1, Pros1, Tnfrsfla, Tgm1, Socs3, Rplp0, Osmr, Cebpd, Tspan7, Srxn1, Capg, Ahnak, Fgf13, S100a10, Arpc1b, Slc6a1, Lgals3, Bcl3, Plin2, Csf2rb2, Clic1, Gfap, Aspg, Emp1, and/or Msr1 are detected (e.g., quantified) by performing RNA sequencing (RNAseq) on the biological sample (e.g., RNA isolated from the biological sample). In some embodiments, the psi (Ψ) score for each of the one or more detected (e.g., quantified) genes is calculated by performing a mixture of isoforms (MISO) analysis.

In some embodiments, the subject is characterized as being a candidate for having pre-symptomatic ALS/FTD based on the presence, in a biological sample obtained from the subject, of a C9orf72 expansion repeat greater than 30 repeats (e.g., 31, 32, 33, 34, 35, 50, 75, 100, or more repeats) or wherein the subject expresses one or more RAN proteins from a C9orf72 expansion repeat. In some embodiments, the control sample, to which the psi (Ψ) score calculated from the sample taken from the subject (e.g., a human or a mouse) having a C9orf72 expansion repeat greater than 30 repeats (e.g., 31, 32, 33, 34, 35, 50, 75, 100, or more repeats) and/or RAN proteins translated from a C9orf72 expansion repeat is compared, is a biological sample obtained from a healthy subject (e.g., a human or a mouse without disease). In some embodiments, the control sample is a biological sample obtained from the same subject at an earlier point in time.

In some embodiments, a therapeutic agent (e.g., a peptide, protein, nucleic acid, small molecule) is administered to a subject identified as having pre-symptomatic or acute ALS/FTD using the methods described herein. In some embodiments, the peptide is a peptide vaccine that targets a RAN protein. In some embodiments, the protein is an antibody. In some embodiments, the antibody is an anti-RAN protein antibody. In some embodiments, the anti-RAN protein antibody binds to a di-amino acid repeat region of a RAN protein. In some embodiments, the therapeutic agent targets a DNA repair pathway gene or gene product. In some embodiments, the therapeutic agent targets one or more gene(s) identified by the method of the present disclosure. In some embodiments, the therapeutic agent targets a protein or RNA which is expressed or encoded by one or more gene(s) identified by the method of the present disclosure.

In some embodiments, the subject identified as having pre-symptomatic or acute ALS/FTD using the methods described herein undergoes treatment for ALS/FTD. In some embodiments, treatment comprises administering an effective amount of a known ALS therapeutic agent, such as Riluzole (Rilutek, Sanofi-Aventis), to a subject identified as having ALS. In some embodiments, treatment comprises administering an effective amount of a known FTD therapeutic agent, such as trazodone (Desyrel, Oleptro) or a selective serotonin reuptake inhibitor (SSRI), to a subject identified as having FTD. In some embodiments, treatment comprises administering an effective amount of a therapeutic agent, such as baclofen, diazepam, phenytoin, trihexyphenidyl and/or amitriptyline, which reduces one or more symptoms of ALS or FTD in a subject identified as having ALS or FTD.

An effective amount is a dosage of a therapeutic agent sufficient to provide a medically desirable result, such as treatment of ALS or FTD. The effective amount will vary with the age and physical condition of the subject being treated, the severity of ALS or FTD in the subject, the duration of the treatment, the nature of any concurrent therapy, the specific route of administration and the like factors within the knowledge and expertise of the health practitioner.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1E show characterization of transgene integration site, RAN protein expression and C9orf72 protein levels in C9-BAC mice. FIG. 1A shows a BAC construct containing human C9orf72 gene and 51.6 kb and 19.4 kb of upstream (left) and downstream (right) flanking sequence, respectively. FIG. 1B is a map showing breakpoint and integration of the BAC in an intergenic region on mouse chromosome 6. FIG. 1C shows a protein blot probed with α-C9ORF72 antibody. FIG. 1D shows protein blots probed with α-C90RF72 antibody. FIG. 1E shows protein blot quantification; statistical analyses were performed using one way ANOVA with Bonferroni correction for multiple comparisons, with Mean ± SEM shown, not significant (ns) = p > 0.05.

FIGS. 2A-2G show earlier onset and increased penetrance in isogenic C9 sublines with longer G₄C₂ expansions. FIG. 2A is a schematic showing the breeding strategy of expansion and contraction lines established from the C9-500 animals with spontaneous intergenerational instability. FIG. 2B shows southern blots of tail and brain DNA from F3 C9-500 mice and C9-800 and C9-50 sublines. FIG. 2C shows gait abnormalities in C9-800 mice at 3 months of age; non-transgenic (NT), n=44, C9-500, n=38, C9-800, n=24. FIG. 2D shows open field analyses comparing the percentage change in ambulatory distance in C9-50, C9-500, and C9-800 mice compared to NT mice at 24 weeks (NT, n=9; C9-50, n=10; C9-500, n=9; C9-800, n=13) and 40 weeks (NT, n=9; C9-50, n=6; C9-500, n=18; C9-800, n=10). Statistical analyses were performed using one-way ANOVA and Tukey’s multiple comparison test with mean +/- SEM shown; not significant (ns) = p > 0.05; **p<0.01; ****p <0.0001; ****p <0.0001. FIG. 2E shows open field analyses showing the percentage of time spent in the center for NT, C9-50, C9-500, and C9-800 mice (NT, n=9; C9-50, n=6; C9-500, n=18; C9-800, n=10). Statistical analyses were performed using one-way ANOVA and Tukey’s multiple comparison test with mean +/- SEM shown; not significant (ns) = p > 0.05; **p<0.01; ****p <0.0001; ****p <0.0001. FIG. 2F shows population census with percentage of female mice that are sick, healthy, and phenotypic using multifactorial scoring criteria. Chi square test. ****p<0.0001. FIG. 2G shows the Kaplan-Meier survival curves of females from NT (n=31), C9-50 (n=23), C9-500 (n=36) and C9-800 (n=52) cohorts; Gehan-Breslow-Wilcoxon test, *p < 0.05.

FIGS. 3A-3E show that molecular features of C9orf72 ALS/FTD increase with increase in repeat length. FIG. 3A depicts quantification of fluorescence in situ hybridization (FISH) detection of sense RNA foci in the dentate gyrus of the hippocampus. FIG. 3B shows quantification of fluorescence in situ hybridization (FISH) detection of antisense RNA foci in the dentate gyrus of the hippocampus. FIG. 3C shows representative images of GA and GP RAN protein aggregates (circled) in the retrosplenial cortex of 40-week old female C9-500 and C9-800 mice. FIGS. 3D and 3E show quantification of GA (FIG. 3D) and GP (FIG. 3E) RAN protein aggregates that was done in a blinded fashion and is shown as the percentage of neurons with aggregates in non-transgenic (NT), C9-50, C9-500, and C9-800 mouse cohorts. Statistical analyses were done using a one way ANOVA with a Bonferroni correction for multiple comparisons, Mean ± SEM, *p < 0.05, **p < 0.01, ****p < 0.0001.

FIGS. 4A-4D show prominent neurodegenerative and neuroinflammatory changes in acute end-stage C9-BAC mice. FIG. 4A depicts a sample-to-sample correlation plot showing increased correlation in gene expression changes between acute mice and C9(+) pre-symptomatic mice, but not between the groups. FIG. 4B shows gene ontology analyses of gene expression changes in acute vs. non-transgenic (NT) mice. FIG. 4C shows cell type enrichment analyses in NT, C9(+) pre-symptomatic, and acute mice. Statistical tests and significance are shown in Table 3. FIG. 4D shows immunohistochemistry of acute C9-BAC mice stained with neuronal marker (NeuN; top row) and microglial marker (Iba1; middle row). Inset shows zoom-in of microglial staining (bottom row). Acute middle and right columns represent earlier and later disease stages in C9-BAC mice, respectively.

FIGS. 5A-5F show abundant alternative splicing changes in C9-BAC mice. FIG. 5A depicts a Venn diagram showing a number of alternative splicing changes in acute and pre-symptomatic mice as compared to non-transgenic (NT) mice. FIG. 5B shows that alternative splicing events found in both pre-symptomatic and acute animals show delta psi values for a group of markers increase with disease severity. FIG. 5C shows Elavl2 is alternatively spliced in both the C9(+) pre-symptomatic and acute mice. FIG. 5D shows gene IDs and corresponding Δpsi values in C9(+) pre-symptomatic and acute animals. Additional information on alternative splicing events is shown in Table 4. FIG. 5E shows gene ontology categories for alternatively spliced events found only in: C9(+) pre-symptomatic mice only, both pre-symptomatic and acute animals (intersection), acute animals only, and C9-ALS patients. FIG. 5F shows a motif analysis of alternative splicing events in pre-symptomatic C9(+) mice, acute C9(+) mice, and C9-ALS patients that demonstrates enrichment of motifs.

FIG. 6 shows transcriptome changes during disease progression. Acute end-stage C9-BAC mice are characterized by neurodegeneration and gene expression changes, while pre-symptomatic mice are characterized by fewer gene expression changes and no overt neurodegeneration. In contrast, the upper panel shows that both symptomatic and pre-symptomatic mice are characterized by alternative splicing changes that increase with an increase in disease severity. Similar to acute C9-BAC mice, C9-ALS patients are also characterized by neurodegenerative changes, gene expression, and alternative splicing changes. Based on these changes and similarities between human and mouse molecular and behavioral phenotypes, biomarkers indicating an early predisposition to disease can be determined.

FIGS. 7A-7C show analysis of transgene integration sites in the C9-500/32 line. FIG. 7A is a schematic diagram of transgene integration in the C9-500/32 line. FIG. 7B shows schematic diagrams indicating the possible location of GGGGCC repeats with 500 or 32 copies (SEQ ID NOs: 2 and 1, respectively). FIGS. 7A and 7B show the human transgene sequence upstream, asterisk marked lines (*), and downstream, pound marked lines (#), of the expansion mutation. Caret marked lines (^) denote mouse chromosomal regions. FIG. 7C shows a southern blot of genomic DNA digested with BglII, which demonstrates that the larger repeat was integrated into the full-length transgene as predicted in possible integration event I (as shown in FIG. 7B).

FIGS. 8A-8D show transgene integration in C9-36/29 and C9-37 lines. FIG. 8A is a schematic diagram showing the transgene integration site in the C9-36/29 line, which contains 4 copies of BAC transgene in the Mppe1 gene. FIG. 8B is a schematic diagram showing the location of the transgene within Mppe1; qPCR shows no change in expression of the Mppe1 gene upon transgene integration. FIG. 8C depicts RNA sequencing of C9-36/29 mice, which shows no change in coverage over the Mppe1 gene. FIG. 8D is a schematic diagram showing the site of transgene integration in C9-37 mice. FIGS. 8A and 8D show the human transgene sequence upstream, asterisk marked lines (*), and downstream, pound marked lines (#), of the expansion mutation, respectively. Caret marked lines (^) denote mouse chromosomal regions.

FIGS. 9A-9C show RNA and RAN protein levels in C9-BAC transgenic mice. FIG. 9A depicts RT-qPCR showing levels of exonla containing transcripts in C9-BAC transgenic lines. FIG. 9B shows the use of an MSD immunoassay to measure levels of soluble GP in cortical brain lysates. FIG. 9C shows the use of an MSD immunoassay to measure levels of soluble GP in cerebellar brain lysates. Statistical analyses were done using a one-way ANOVA with multiple comparison, Bonferroni correction, Mean ± SEM, *p<0.05, **p<0.01, ****p <0.0001.

FIGS. 10A-10D show molecular changes in allelic series sublines. FIG. 10A shows representative images of GA and GP aggregates (circled) in the retrosplenial cortex of C9 BAC mice. FIG. 10B shows the quantification of GA aggregates measured in the retrosplenial cortex at 20 weeks of age. FIG. 10C shows quantification of GP aggregates measured in the retrosplenial cortex at 20 weeks of age. FIG. 10D shows the use of an MSD immunoassay to measure levels of soluble GP in brain lysates at 40 weeks in allelic series sublines. Statistical analyses were done using a one-way ANOVA with multiple comparison, Bonferroni correction, Mean ± SEM, *p<0.05, **p<0.01, ****p <0.0001.

FIG. 11 shows transcriptome changes in acute C9-BAC mice. Heat map shows the top 50 differentially expressed genes in acute vs. non-transgenic (NT) mice, and the relative expression of those genes in NT and C9(+) pre-symptomatic mice.

FIG. 12 shows differential gene expression changes in acute vs. non-transgenic (NT) mice, and C9-ALS patients vs. controls. The Venn diagram shows overlap between differential gene expression that was measured from acute vs. NT mice, and C9-ALS patients vs. controls. The genes from the intersection category are listed.

FIG. 13 shows that no overt neuropathology was seen in C9(+) pre-symptomatic animals. Cresyl violet staining of the hippocampus from three C9(+) pre-symptomatic animals is shown.

FIG. 14 shows motif analysis from an alternative splicing dataset obtained from transverse abdominal (TA) muscle of DM1 patients.

FIGS. 15A-15F show single-copy C9-500 mice that express higher GP levels than mixed repeat length, high-copy Baloh-Jax mice. FIG. 15A is a schematic of the transgene used to generate C9-13 Baloh-Jax C9-BAC mice. FIG. 15B is a southern blot in C9-LCL, C9-50, C9-500, non-transgenic (NT), and Baloh-Jax C9-BAC mice showing multiple copies of transgene in the Baloh-Jax mice. FIG. 15C is a map of three transgene integration events found in Baloh-Jax C9-BAC mice. FIG. 15D is a table listing the total number of copies of transgene and the pCC1BAC backbone identified by bioinformatics analyses. FIG. 15E shows RNA levels of repeat-containing sense transcripts in NT, C9-500, and Baloh-Jax mice measured by qRT-PCR; one way ANOVA with multiple comparisons, Bonferroni correction, Mean + SEM, ****p < 0.0001. FIG. 15F shows an MSD immunoassay to measure soluble GP RAN protein in cortex and cerebellum in NT, C9-500, and Baloh-Jax C9-BAC mice; one way ANOVA with multiple comparisons, Bonferroni correction, Mean + SEM, **p < 0.01, ****p < 0.0001, ns p > 0.05.

FIGS. 16A-16C show Vgll4 expression in non-transgenic (NT) animals, or animals with C9-50, 500 or 800 repeats. FIG. 16A is a schematic showing the location of the transgene in Vgll4 gene, and qRT-PCR comparing expression levels of Vgll4 in NT and C9-500 mice. FIG. 16B depicts a histogram showing coverage of reads over the first exon and intron of Vgll4 gene. The bar graph shows the normalized counts from the RNA sequencing data set (n=3 animals per group). qRT-PCR and RNA sequencing showed no apparent differences in the levels of Vgll4 in C9-500 compared to NT mice. FIG. 16C depicts qRT-PCR, which shows expression of Vgll4 relative to β-actin. qRT-PCR shows that Vgll4 expression was not different in animals with C9-50, 500 or 800 repeats.

### DETAILED DESCRIPTION

In some aspects, the disclosure relates to methods for the diagnosis and treatment of amyotrophic lateral sclerosis (ALS) and frontotemporal dementia (FTD). The disclosure is based, in part, on the recognition that isoforms of certain genes as described herein undergo alternative splicing changes that are detectable (e.g., quantifiable) in a biological sample obtained from a subject having or at risk of having pre-symptomatic ALS/FTD. In some embodiments, a biological sample comprises blood, serum, and/or tissue (e.g., cerebrospinal fluid (CSF) or central nervous system (CNS) tissue). In some embodiments, the isoforms of the certain genes are those described the present disclosure.

### Amyotrophic Lateral Sclerosis (ALS) and Frontotemporal Dementia (FTD)

Amyotrophic lateral sclerosis (ALS) is a debilitating disease with varied etiology characterized by rapidly progressing weakness, muscle atrophy, muscle spasticity, difficulty speaking (dysarthria), difficulty swallowing (dysphagia), and difficulty breathing (dyspnea). Although the order and rate of symptoms varies from person to person, eventually most subjects are not able to walk, get out of bed on their own, or use their hands and arms. Most subjects with ALS will eventually die from respiratory failure, usually within three to five years from the onset of symptoms. Riluzole (Rilutek) is a currently available treatment for ALS, but only slows progression and increases survival to a modest extent. Frontotemporal dementia (FTD) is also a devastating group of disorders resulting from atrophy or shrinkage of the frontal and temporal lobes of the brain. This shrinkage or atrophy results in severe behavioral changes.

The intronic C9orf72 GGGGCC (G₄C₂) hexanucleotide microsatellite repeat expansion mutation is a commonly known genetic cause of ALS and FTD. The 5′-GGGGCC-3′ and 5′GGCCCC-3′ hexanucleotide repeat-containing RNAs comprise a repeat nucleic acid sequence of the formula (GGGGCC)ₓ or (GGCCCC)ₓ, respectively, where X may be at least 10, at least 20, at least 25, at least 30, at least 50, at least 100, or at least 1,000, or in a range selected from 10-100,000, 10-50,000, 10-5,000, 20-1,000, 20-100,000, 20-50,000, 20-5,000, 20-1,000, 25-100,000, 25-50,000, 25-5,000, or 25-1,000 (SEQ ID NO: 3). There is remarkable clinical heterogeneity among C9orf72 expansion carriers, with clinical presentation ranging from the muscle wasting disease of ALS found in some patients to the disinhibition and cognitive deficits characteristic of FTD in others. Alternative splicing changes common to acute ALS/FTD and C9-positive pre-symptomatic ALS/FTD cases have not been thoroughly characterized.

Aspects of the disclosure relate to methods for detecting (e.g., quantifying) changes in alternative splicing of certain genes disclosed herein in a subject or in a biological sample obtained from a subject. In some embodiments, the methods described by the disclosure are useful for the diagnosis and/or treatment of subjects who have, or are suspected of having, ALS/FTD.

In some embodiments, a subject is a mammal. In some embodiments, a mammal is a mouse, rat, non-human primate, or human. In some embodiments, one or more changes in alternative splicing are detected (e.g., quantified) in a cell or a culture of cells.

In some embodiments, the subject is a healthy subject. As used herein, a healthy subject is a subject that does not exhibit signs or symptoms of ALS/FTD. In some embodiments, a healthy subject has a psi score of zero (0) for certain isoforms of certain genes as described herein (e.g., as shown in Table 4). In some embodiments, a healthy subject is a subject that has 25 or fewer GGGGCC (SEQ ID NO: 3) hexanucleotide repeats within a C9orf72 gene.

In some embodiments, a subject of the present disclosure has, and/or has been diagnosed as having, the acute form of ALS/FTD. The acute form of ALS/FTD can be C9-positive or C9-negative. As used herein, “subjects having the acute form of ALS/FTD” (C9-positive or C9-negative) refers to those subjects that exhibit one or more visible, noticeable, or otherwise phenotypic symptoms of ALS/FTD (e.g., muscle twitching, cramping, stiffness, or weakness, difficulty speaking and/or breathing, memory loss, etc.). Thus, in some embodiments, the subject is diagnosed as having the acute form of ALS/FTD (C9-positive or C9-negative) based upon the presence of one or more visible, noticeable, or otherwise phenotypic symptoms of ALS/FTD.

In some embodiments, the subject is suspected of having the acute form of ALS/FTD (C9-positive or C9-negative). As used herein, “subjects suspected of having the acute form of ALS/FTD” refers to those subjects who have not yet been formally diagnosed as having ALS/FTD (e.g., by a medical doctor), but who are nonetheless suspected of having the disease. In some embodiments, the subject is suspected of having the acute form of ALS/FTD (C9-positive or C9-negative) based upon an established family history of ALS/FTD. In some embodiments, a subject suspected of having the acute form of ALS/FTD exhibits one or more signs or symptoms associated with ALS/FTD, and/or has one or more mutations in a gene associated with ALS/FTD, e.g., C90rf72, SOD1, etc. In some embodiments, a subject suspected of having the acute form of ALS/FTD does not exhibit one or more signs or symptoms associated with ALS/FTD.

In some embodiments, a subject of the present disclosure has, or has been diagnosed as having, the pre-symptomatic form of C9-positive ALS/FTD. As used herein, “subjects having the pre-symptomatic form of C9-positive ALS/FTD” refers to those subjects that are not yet exhibiting visible, noticeable, or otherwise phenotypic symptoms of C9-positive ALS/FTD, but who, in the absence of phenotypic symptoms, have been diagnosed with C9-positive ALS/FTD (e.g., by a medical doctor) using certain genetic markers known in the art to be associated with C9-positive ALS/FTD. In some embodiments, the subject is suspected of having the pre-symptomatic form of C9-positive ALS/FTD. In some embodiments, the subject is suspected of having the pre-symptomatic form of C9-positive ALS/FTD based upon an established family history of ALS/FTD.

As used herein, “a subject having, or suspected of having, C9-positive ALS/FTD” (acute or pre-symptomatic) refers to a subject who has one or more mutations in a gene associated with ALS/FTD, e.g., C90rf72, SOD1, etc. Thus, in some embodiments the subject is diagnosed as having the acute form or the pre-symptomatic form of C9-positive ALS/FTD based upon the detection of a heterozygous C9orf72 pathogenic GGGGCC (G₄C₂) hexanucleotide repeat expansion using molecular genetic testing techniques (e.g., genotyping, sequencing, amplifying/hybridizing, etc.). In some embodiments, the repeat expansion is greater than 30 repeats (e.g., 31, 32, 33, 34, 35, 50, 75, 100, or more repeats) (SEQ ID NO: 3). In some embodiments, the subject is diagnosed as having the acute form or the pre-symptomatic form of C9-positive ALS/FTD based upon the method of diagnosis described in US Patent No. 10,295,547, incorporated by reference herein in its entirety.

In some embodiments, the subject is diagnosed as having acute or pre-symptomatic ALS/FTD based on a level of one or more di-amino acid repeat-containing proteins in a sample (e.g., blood, serum, or tissue) obtained from a subject. In some embodiments, the di-amino acid repeat-containing proteins are selected from the group consisting of: poly-(Gly-Ala), poly-(Gly-Pro), poly-(Gly-Arg), poly-(Pro-Ala), poly-(Pro-Arg), Met ... poly-(Pro-Arg) or Met ... poly-(Gly-Pro). In some embodiments, a level of the one or more di-amino acid-repeat-containing proteins that is elevated in the sample compared to a control level indicates that the subject has C9-positive ALS or FTD (e.g., a “positive sample”). In some embodiments, a positive sample comprises a number of di-amino acid repeat-containing proteins present in the sample, for example 30, 50, 100, or 1,000 di-amino acid repeat-containing proteins. Each di-amino acid repeat-containing protein comprises a repeat amino acid sequence, which contains a di-amino acid repeat unit of the formula (YZ)ₓ, where X can be from 2-10,000, 5-10,000, 2-5,000, 5-5,000, 2-1000, 5-1000, 5-500, 5-300, 5-200, 10-500, 10-300, or 10-200.

In the absence of one or more mutations in a gene associated with ALS/FTD, e.g., C90rf72, SOD1, etc. and/or in the absence of phenotypic symptoms of ALS/FTD, diagnosing a subject as having ALS/FTD is difficult. However, early diagnosis (e.g., pre-symptomatic diagnosis) is desirable to allow for early treatment intervention, which may slow or prevent disease onset. Aspects of the instant disclosure provide methods to identify and diagnose pre-symptomatic subjects as having ALS/FTD.

Thus, in some embodiments, a subject of the present invention has, or has been diagnosed as having, the pre-symptomatic form of C9-negative ALS/FTD. As used herein, “subjects having the pre-symptomatic form of C9-negative ALS/FTD” refers to those subjects that are not yet exhibiting visible, noticeable, or otherwise phenotypic symptoms of C9-negative ALS/FTD (e.g., muscle twitching, cramping, stiffness, or weakness, difficulty speaking and/or breathing, memory loss, etc.). In some embodiments, the subject is suspected of having the pre-symptomatic form of C9-negative ALS/FTD. In some embodiments, the subject is suspected of having the pre-symptomatic form of C9-negative ALS/FTD based upon an established family history of ALS/FTD.

Accordingly, in some embodiments, the subject not exhibiting visible, noticeable, or otherwise phenotypic symptoms of ALS/FTD, and not carrying the repeat expansions or di-amino acid repeat-containing proteins associated with C9orf72 as described herein, may have pre-symptomatic C9-negative ALS/FTD.

Subjects having a pre-symptomatic form of ALS/FTD may nonetheless be carrying alternative genetic markers of the disease. However, other genetic markers (e.g., those not encompassed by the detection of a C9orf72 pathogenic GGGGCC (G₄C₂) hexanucleotide repeat expansion or the associated di-amino acid repeat containing proteins) that can reliably identify a subject as having or likely to develop ALS/FTD are as-yet uncharacterized in the art. The present disclosure surprisingly identifies genetic markers that may be useful in diagnosing a pre-symptomatic subject as having ALS/FTD.

### Isoforms

Aspects of the disclosure relate to the recognition that detecting (e.g., quantifying) changes in alternative splicing of certain genes as described herein (e.g., by detecting the presence or absence of certain isoforms of those genes, or by quantifying changes in expression levels of said isoforms) allows for identification of (1) a subject having pre-symptomatic ALS/FTD, and/or (2) the progression of pre-symptomatic ALS/FTD to symptomatic ALS/FTD in a subject. As used herein, an “isoform” means a particular variant of a gene, mRNA, cDNA, or the protein encoded thereby, distinguished from other variants by its particular sequence and/or structure. It will be understood by those skilled in the art that genes can express two or more isoforms, and that these two or more isoforms can result from alternative splicing of pre-mRNA, alternative transcription initiation and/or termination, alternative translation initiation and/or termination, and/or other disruptions that can occur in gene expression. In some embodiments, two isoforms of a gene share between about 50% and 99% identity at the amino acid level, for example 50% identity, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or any value encompassed therein, for example 50.1%, 50.01%, 50.001%, etc. identity.

In some embodiments, the disclosure relates to the detection (e.g., quantification) of an increase or decrease in the level of certain isoforms of certain genes of a subject, as described herein, that, when detected, can be used as an early biomarker for ALS/FTD. In some embodiments, isoforms of one or more genes, for example 1, 2, 3, 4, 5, 10, 20, 50, 100, 200, 500, etc. genes, are detected (e.g., quantified). In some embodiments, isoforms of one or more combinations, for example, 1, 2, 3, 4, etc. combinations, of one or more genes, for example 1, 2, 3, 4, 5, 10, 20, 50, 100, 200, 500, etc. genes, are detected (e.g., quantified).

In some embodiments, isoforms of genes, for example Nek6, Pphln1, Pdgfc, Pomt1, Sorbs1, Ssfa2, Rps6kb2, Irak1, Cpeb4, Calu, Gtpbp10, Pard3, Ranbp3, Prx, Rad18, 2810474O19Rik, Mtdh, Sema6d, Arl6, Tbp, Slx4, Abi1, Osbpl1a, Pex7, Camklg, Idnk, Zfp963, Firre, Grik1, Ktn1, Nmbr, Sec14l2, Gm21992, Mpnd, Elavl2, Mtmr1, Six3os1, Tenm4, Zyx, Camsap3, Brat1, Hdac5, Fmnl3, Pisd, Atp11c, Lair1, Ccnc, Nnat, Fam111a, Mettl4, D130020L05Rik, Etfrf1, Chd2, Ikzf4, Phkb, A730017C20Rik, Tnrc6a, Itga3, Gprasp1, Ptprf, Cd200, Dync1i2, Huwe1, Aplp2, Ctbs, Mink1, Dpy19l4, Ccdc85a, Asph, Nr3c2, Kif2a, Dennd5a, Pip5klc, Plxna3, Arhgef9, Iqsec2, Fyttd1, Smyd4, Tmem234, Aif1l, Adgrl3, 0610037L13Rik, 1600014C10Rik, 1700001L05Rik, 3010001F23Rik, 6430573F11Rik, 9030617O03Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcrl, Camkk2, Ccdc43, Ccdc66, Cd180, Cdk14, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Dcaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4g1, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Gains, Glt8d1, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcnt1, Klc4, Las1l, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf4l2, Morn1, Mpv17l2, Mthfsl, Myole, Myo6, Myrf, Myt1, N6amt1, Naa35, Nav3, Nckap5, Ncoa1, Ncor1, Ndor1, Necap2, Nemp2, Nf1, Nfasc, Nfia, Nful, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pcdh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfia1, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassf2, Rbm10, Rbm12, Rbm39, Rbms3, Rcbtb2, Retreg3, Rffl, Rhobtb1, Rhoj, Ripor2, Rpain, Rpgr, Rps24, Rps6ka1, Rtel1, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1, Snap23, Snrpa1, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd52l2, Trp53i13, Trpc1, Ttc17, Ttc21b, Ttll5, Tysnd1, Ube2cbp, Ubn2, Ubxn7, Uhrf1bp1, Unc13b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Spp1, Plek, Col5a3, Slpr3, Anxa2, Mt1, Sbno2, Csf1, Ch25h, Mt2, Serpine1, Vim, Col16a1, Neat1, Cd109, Dysf, C3ar1, Pros1, Tnfrsf1a, Tgm1, Socs3, Rplp0, Osmr, Cebpd, Tspan7, Srxn1, Capg, Ahnak, Fgƒ13, S100a10, Arpc1b, Slc6a1, Lgals3, Bcl3, Plin2, Csf2rb2, Clic1, Gfap, Aspg, Emp1, and/or Msr1 are detected (e.g., quantified).

### Methods

In some aspects, the disclosure relates to methods for detecting (e.g., quantifying) a change (e.g., an increase or decrease) in expression of the certain isoforms of one or more genes as described herein (e.g., genes associated with ALS/FTD). In some aspects, the disclosure relates to methods for detecting the presence of (e.g., the presence or absence of) certain isoforms of one or more genes as described herein (e.g., genes associated with ALS/FTD).

As used herein, “detecting” a change in expression or “detecting” a level of certain isoforms of one or more genes comprises “quantifying” an expression level or amount of a gene isoform transcript (e.g., an mRNA transcript) encoding a protein isoform(s) and/or quantifying the expression level or amount of a protein isoform directly. As used herein, “detecting” can also embrace methods of detecting the presence or absence of a gene or protein isoform of interest, with or without the additional step of quantifying the expression level of said isoform. As will be understood by the skilled artisan, detecting a change in expression of an isoform, or detecting a level of an isoform, in some embodiments requires a step of quantification. In embodiments where a change in expression levels is detected, at least two steps of quantification are performed, so as to quantify the change in expression level of the gene or protein isoform. Thus, as used herein, “detecting” may be used interchangeably to refer to detecting the presence or absence of an isoform, in some embodiments, and/or to quantifying an expression level or amount of an isoform, in some embodiments.

In some embodiments, detecting (e.g., quantifying) comprises performing RNA sequencing (RNA-seq) on the biological sample (e.g., RNA isolated from the biological sample). RNA-seq utilizes next generation sequencing (NGS) to examine the quantity and sequences of RNA that are present in a sample. Thus, in some embodiments, performing RNA-seq comprises NGS. In some embodiments, detecting (e.g., quantifying) comprises performing northern blot analysis. In some embodiments, detecting (e.g., quantifying) comprises performing a nuclease protection assay (NPA). In some embodiments, detecting (e.g., quantifying) comprises performing in situ hybridization. In some embodiments, detecting (e.g., quantifying) comprises performing reverse transcription-polymerase chain reaction (RT-PCR). In some embodiments, detecting (e.g., quantifying) comprises performing enzyme-linked immunosorbent assay (ELISA) assay. In some embodiments, detecting (e.g., quantifying) comprises performing western blot analysis. In some embodiments, detecting (e.g., quantifying) comprises performing mass spectrometry.

In some embodiments, two or more isoforms of one or more genes are detected (e.g., quantified). In some embodiments, the two or more isoforms of one or more genes are selected from: Nek6, Pphln1, Pdgfc, Pomt1, Sorbs1, Ssfa2, Rps6kb2, Irak1, Cpeb4, Calu, Gtpbp10, Pard3, Ranbp3, Prx, Rad18, 2810474019Rik, Mtdh, Sema6d, Arl6, Tbp, Slx4, Abi1, Osbpl1a, Pex7, Camklg, Idnk, Zfp963, Firre, Grik1, Ktn1, Nmbr, Sec14l2, Gm21992, Mpnd, Elavl2, Mtmr1, Six3os1, Tenm4, Zyx, Camsap3, Brat1, Hdac5, Fmnl3, Pisd, Atp11c, Lair1, Ccnc, Nnat, Fam111a, Mettl4, D130020L05Rik, Etfrf1, Chd2, Ikzƒ4, Phkb, A730017C20Rik, Tnrc6a, Itga3, Gprasp1, Ptprf, Cd200, Dyncli2, Huwe1, Aplp2, Ctbs, Mink1, Dpy19l4, Ccdc85a, Asph, Nr3c2, Kif2a, Dennd5a, Pip5k1c, Plxna3, Arhgej9, Iqsec2, Fyttd1, Smyd4, Tmem234, Aif1l, Adgrl3, 0610037L13Rik, 1600014C10Rik, 1700001E05Rik, 3010001F23Rik, 6430573F11Rik, 9030617003Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgej7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcrl, Camkk2, Ccdc43, Ccdc66, Cd180, Cdk14, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Deaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4g1, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Galns, Glt8d1, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcnt1, Klc4, Las1l, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf412, Morn1, Mpv17l2, Mthfsl, Myo1e, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pcdh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfial, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassj2, Rbm10, Rbm12, Rbm39, Rbms3, Rcbtb2, Retreg3, Rffl, Rhobtb1, Rhoj, Ripor2, Rpain, Rpgr, Rps24, Rps6ka1, Rtel1, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50al, Smoc1, Snap23, Snrpa1, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd5212, Trp53i13, Trpc1, Ttc17, Ttc21b, Ttll5, Tysnd1, Ube2cbp, Ubn2, Ubxn7, Uhrf1bp1, Unc13b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Spp1, Plek, Col5a3, S1pr3, Anxa2, Mt1, Sbno2, Csf1, Ch25h, Mt2, Serpine1, Vim, Col16a1, Neat1, Cd109, Dysf, C3ar1, Pros1, Tnfrsf1a, Tgm1, Socs3, Rplp0, Osmr, Cebpd, Tspan7, Srxn1, Capg, Ahnak, Fgƒ13, S100a10, Arpc1b, Slc6a1, Lgals3, Bcl3, Plin2, Csf2rb2, Clic1, Gfap, Aspg, Emp1, and/or Msr1.

In some embodiments, the two or more isoforms of one or more genes selected from the above list are detected (e.g., quantified) in a biological sample. In some embodiments, the biological sample is a blood sample, serum sample, or a tissue sample. In some embodiments, the tissue sample is a central nervous system (CNS) tissue sample or a cerebrospinal fluid (CSF) sample. In some embodiments, the biological sample is obtained from a subject that has or is suspected of having pre-symptomatic C9-negative ALS/FTD or pre-symptomatic C9-positive ALS/FTD.

The level of isoforms present in a sample taken from a subject may be assessed on an absolute basis or as, e.g., an increase or a decrease in expression on a relative basis. Assessment of an increase or decrease on a relative basis may be made as a fold change, for example one-fold (e.g., one-fold more or one-fold less), two-fold, three-fold, four-fold, five-fold, six-fold, seven fold-eight-fold, nine-fold, ten-fold, fifteen-fold, twenty-fold, thirty-fold, forty-fold, fifty-fold, sixty-fold, seventy-fold, eighty-fold, ninety-fold, one hundred-fold, one-thousand-fold, etc. change, or as a percent change, for example, 1% (e.g., 1% more or 1% less), 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 250%, 300%, 400%, 500%, 1,000%, etc. change. When assessed on a relative basis, comparison may be made to controls, including, but not limited to, a historical sample from the same patient (e.g., serial samples, longitudinal samples); level(s) found in a patient or population of patients absent of disease or disorder (e.g., a “healthy” subject); a threshold value; an acceptable range; etc.

Therefore, in some embodiments, the increase or decrease in expression of the isoforms is relative to a control sample. In some embodiments, a control sample is a prior sample screened in the same subject having or suspected of having pre-symptomatic C9-negative ALS/FTD or pre-symptomatic C9-positive ALS/FTD (e.g., a sample taken from the same subject 1 hour earlier than the second sample, 1 day earlier, 2 days earlier, 3 days earlier, 4 days earlier, 5 days earlier, 6 days earlier, 1 week earlier, 2 weeks earlier, 3 weeks earlier, 1 month earlier, 2 months earlier, 3 months earlier, 6 months earlier, 1 year earlier, 2 years earlier, 3 years earlier, 4 years earlier, 5 years earlier, 10 years earlier, 20 years earlier, etc.). In some embodiments, a control sample is a later sample screened in the same subject having or suspected of having pre-symptomatic C9-negative ALS/FTD or pre-symptomatic C9-positive ALS/FTD (e.g., a sample taken from the same subject 1 hour later than the first sample, 1 day later, 2 days later, 3 days later, 4 days later, 5 days later, 6 days later, 1 week later, 2 weeks later, 3 weeks later, 1 month later, 2 months later, 3 months later, 6 months later, 1 year later, 2 years later, 3 years later, 4 years later, 5 years later, 10 years later, 20 years later, etc.).

In some embodiments, the control sample is taken from a subject who has not been diagnosed with, and has no visible, noticeable, or otherwise phenotypic symptoms of, ALS/FTD (e.g., a healthy control subject).

In some embodiments, a control sample is a sample taken from a different subject having acute C9-positive ALS/FTD. In some embodiments, a control sample is a sample taken from a different subject having acute C9-negative ALS/FTD. In some embodiments, a control sample is a sample taken from a different subject having pre-symptomatic C9-positive ALS/FTD. In some embodiments, a control sample is a sample taken from a different subject having pre-symptomatic C9-negative ALS/FTD.

In some embodiments, a control sample is a sample taken from a control subject that is matched (e.g., age-matched, gender-matched, etc.) to the subject having or suspected of having pre-symptomatic C9-negative ALS/FTD or pre-symptomatic C9-positive ALS/FTD.

In some embodiments, the step of detecting (e.g., quantifying) an increase or decrease in the level of certain isoforms of certain genes as described herein is performed by calculating a percent spliced-in (psi) score. A psi (Ψ) score is a value between 0 to 1 (e.g., 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0, or any value included therein such as, e.g., 0.001, 0.0001, 0.0001, etc.) that quantifies alternative splicing occurrences present within a sample.

In some embodiments, the Ψ score is calculated by dividing the number of inclusion reads (e.g., the number of alternative splicing events for a gene of interest) by the total number of inclusion reads and exclusion reads (e.g., the number of normal (e.g., non-alternative) splicing events for the gene of interest). Therefore, in some embodiments the Ψ score is calculated according to the following formula for the gene of interest:

$$\text{Ψ} \text{score =} \frac{\text{inclusion reads}}{\text{inclusion reads + exclusion reads}}$$

As will be understood by the skilled artisan, the quantity and/or length of expression reads (e.g., inclusion or exclusion reads) which may be produced and subsequently analyzed according to the methods described herein may be large. Sequencing technologies vary in the length of reads produced, and such reads can range from 20-40 base pairs, 100-500 base pairs, 1000-1500 base pairs, or upward of 100 kilobases, in some embodiments. In some embodiments, an expression read (e.g., an inclusion or exclusion read) comprises about 20-40 base pairs, about 30-50 base pairs, about 40-60 base pairs, about 50-70 base pairs, about 60-80 base pairs, about 70-90 base pairs, about 80-100 base pairs, about 90-110 base pairs, about 100-150 base pairs, about 125-175 base pairs, about 150-200 base pairs, about 175-225 base pairs, about 200-300 base pairs, about 250-350 base pairs, about 300-500 base pairs, about 400-600 base pairs, about 500-700 base pairs, about 600-800 base pairs, about 700-900 base pairs, about 800-1000 base pairs (e.g., 800 base pairs-1 kilobase), about 1000-1500 base pairs, about 1250-1750 base pairs, about 1500-2000 base pairs, about 1750-2250 base pairs, about 2000-4000 base pairs, about 3000-5000 base pairs, about 4000-6000 base pairs, about 5000-10000 base pairs, about 7500-12500 base pairs, about 10000-15000 base pairs, about 1-3 kilobases, about 2-4 kilobases, about 3-5 kilobases, about 4-6 kilobases, about 5-7 kilobases, about 6-8 kilobases, about 7-9 kilobases, about 8-10 kilobases, about 10-15 kilobases, about 12-17 kilobases, about 15-20 kilobases, about 17-22 kilobases, about 20-30 kilobases, about 25-35 kilobases, about 30-50 kilobases, about 40-60 kilobases, about 50-70 kilobases, about 60-80 kilobases, about 70-90 kilobases, about 80-100 kilobases, or about 90-110 kilobases.

In some embodiments, the calculating comprises performing a mixture of isoforms (MISO) analysis. MISO analysis provides an estimate of isoform expression levels within a sample based on a statistical model and assesses confidence in those estimates. In some embodiments, MISO analysis is performed using MISO software (see, e.g., Katz, Y., E. T. Wang, et al. (2010), Analysis and design of RNA sequencing experiments for identifying isoform regulation, Nat Methods 7(12): 1009-1015).

In some embodiments, a Ψ score higher than (>) 0.50 (for example, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0, or any value included therein such as, e.g., 0.5001, 0.50001, etc.) indicates that a greater number of alternative splicing events for the gene of interest are present in the tested sample than the number of regular splicing events. Conversely, in some embodiments a Ψ score lower than (<) 0.50 (for example, 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or any value included therein such as, e.g., 0.499, 0.4999, etc.) indicates that a lower number of alternative splicing events for the gene of interest are present in the tested sample than the number of regular splicing events.

As used herein, delta psi (ΔΨ) score is used to refer to the calculation of the difference between two Ψ scores for a single gene of interest (e.g., at different points in time in the same subject, or at the same or different points in time in two different subjects). The difference between the two calculated Ψ scores is the ΔΨ score. It will be understood that, because a Ψ score may be any value between 0 and 1, as described herein, a ΔΨ score (that is, the difference between the two calculated Ψ scores) may also be any value between 0 and 1 (e.g., 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0, or any value included therein such as, e.g., 0.001, 0.0001, 0.0001, etc.) or any value between 0 and -1 (e.g., 0, -0.01, -0.02, -0.03, -0.04, -0.05, -0.06, -0.07, -0.08, -0.09, -0.10, -0.11, -0.12, -0.13, -0.14, -0.15, -0.16, -0.17, -0.18, -0.19, -0.20, -0.21, -0.22, -0.23, -0.24, -0.25, -0.26, -0.27, -0.28, -0.29, -0.30, -0.31, -0.32, -0.33, -0.34, -0.35, -0.36, -0.37, -0.38, -0.39, -0.40, -0.41, -0.42, -0.43, -0.44, -0.45, -0.46, -0.47, -0.48, -0.49, -0.50, -0.51, -0.52, -0.53, -0.54, -0.55, -0.56, -0.57, -0.58, -0.59, -0.60, -0.61, -0.62, -0.63, -0.64, -0.65, -0.66, -0.67, -0.68, -0.69, -0.70, -0.71, -0.72, -0.73, -0.74, -0.75, -0.76, -0.77, -0.78, -0.79, -0.80, -0.81, -0.82, -0.83, -0.84, -0.85, -0.86, -0.87, -0.88, -0.89, -0.90, -0.91, -0.92, -0.93, -0.94, -0.95, -0.96, -0.97, -0.98, -0.99, or -1.0, or any value included therein such as, e.g., -0.001, -0.0001, -0.0001, etc.). In some embodiments, a ΔΨ score may be expressed as an absolute value, where, for example, the absolute value of -0.1 is 0.1.

Accordingly, in some embodiments, the disclosure provides for a method for identifying or diagnosing a subject as having pre-symptomatic ALS. In some embodiments, the pre-symptomatic ALS is C9-positive. In some embodiments, the pre-symptomatic ALS is C9-negative. In some embodiments, the subject is diagnosed as having pre-symptomatic ALS if the absolute value of the calculated ΔΨ score is not zero (0). In some embodiments, the ΔΨ score is calculated by comparing each Ψ score of two or more isoforms of one or more genes to a Ψ score of the same two or more isoforms of one or more genes from a control sample (as defined herein).

In some embodiments, a ΔΨ score is calculated for two or more isoforms of one or more genes selected from: Nek6, Pphln1, Pdgfc, Pomt1, Sorbs1, Ssfa2, Rps6kb2, Irak1, Cpeb4, Calu, Gtpbp10, Pard3, Ranbp3, Prx, Rad18, 2810474019Rik, Mtdh, Sema6d, Arl6, Tbp, Slx4, Abil, Osbpl1a, Pex7, Camklg, Idnk, Zfp963, Firre, Grik1, Ktn1, Nmbr, Sec14l2, Gm21992, Mpnd, Elavl2, Mtmr1, Six3os1, Tenm4, Zyx, Camsap3, Brat1, Hdac5, Fmnl3, Pisd, Atp11c, Lair1, Ccnc, Nnat, Fam111a, Mettl4, D130020L05Rik, Etfrf1, Chd2, Ikzf4, Phkb, A730017C20Rik, Tnrc6a, Itga3, Gprasp1, Ptprf, Cd200, Dync1i2, Huwe1, Aplp2, Ctbs, Mink1, Dpy19l4, Ccdc85a, Asph, Nr3c2, Kif2a, Dennd5a, Pip5k1c, Plxna3, Arhgej9, Iqsec2, Fyttd1, Smyd4, Tmem234, Aif1l, Adgrl3, 0610037L13Rik, 1600014C10Rik, 1700001E05Rik, 3010001F23Rik, 6430573F11Rik, 9030617003Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcrl, Camkk2, Ccdc43, Ccdc66, Cd180, Cdk14, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Deaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4g1, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Galns, Glt8d1, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcnt1, Klc4, Las1l, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf4l2, Morn1, Mpv17l2, Mthfsl, Myo1e, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pcdh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfia1, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassf2, Rbm10, Rbm12, Rbm39, Rbms3, Rcbtb2, Retreg3, Rffl, Rhobtb1, Rhoj, Ripor2, Rpain, Rpgr, Rps24, Rps6ka1, Rtel1, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1, Snap23, Snrpa1, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd52l2, Trp53i13, Trpc1, Ttc17, Ttc21b, Ttll5, Tysndl, Ube2cbp, Ubn2, Ubxn7, Uhrf1bp1, Unc13b, Uqcc1, Usp15, Usp37, Vav2, Vps13e, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Spp1, Plek, Col5a3, S1pr3, Anxa2, Mt1, Sbno2, Csf1, Ch25h, Mt2, Serpine1, Vim, Col16a1, Neat1, Cd109, Dysf, C3arl, Pros1, Tnfrsf1a, Tgm1, Socs3, Rplp0, Osmr, Cebpd, Tspan7, Srxn1, Capg, Ahnak, Fgƒ13, S100a10, Arpc1b, Slc6a1, Lgals3, Bcl3, Plin2, Csf2rb2, Clic1, Gfap, Aspg, Emp1, and/or Msr1, and is detected (e.g., quantified) in the biological samples taken from one or more subjects. In some embodiments, if the gene is Nek6, Pphln1, Pdgfc, Pomt1, Sprbs1, Ssfa2, Rps6kb2, Cpeb4, Calu, Pard3, Ranpb3, Prx, 2810474019Rik, Mtdh, Arl6, Tbp. Slx4, Osbpl1a, Pex7, Camklg, or Idnk, the ΔΨ score is a negative value. In some embodiments, if the gene is Zfp963, Firre, Gtpbp10, Ktnl, Sec1412, Gm21992, Mpnd, Elavl2, Mtmrl, Zyx, Camsap3, Brat1, Hdac5, Fmnl3, Atp11c, Liar1, Ccnc, Nnat, Fam111a, or Mettl4, the ΔΨ score is a positive value.

Thus, in some embodiments, if the absolute value of the ΔΨ score for any one of the following genes is not zero (0), the subject is diagnosed as having pre-symptomatic ALS/FTD (e.g., pre-symptomatic C9-positive or C9-negative ALS/FTD): Nek6, Pphln1, Pdgfc, Pomt1, Sprbs1, Ssfa2, Rps6kb2, Cpeb4, Calu, Pard3, Ranpb3, Prx, 2810474019Rik, Mtdh, Ar16, Tbp. Slx4, Osbpl1a, Pex7, Camklg, Idnk, Zfp963, Firre, Gtpbp10, Ktnl, Sec1412, Gm21992, Mpnd, Elavl2, Mtmrl, Zyx, Camsap3, Brat1, Hdac5, Fmnl3, Atp11c,Liar1, Ccnc, Nnat, Fam111a, or Mettl4.

In some aspects, the disclosure provides a method for monitoring disease progression in a single subject by calculating ΔΨ in one or more genes of a subject, calculated as described herein, in two biological samples taken from the same subject at two different points in time. In some embodiments, the method comprises detecting (e.g., quantifying) in a first biological sample obtained from the subject at a first point in time a first Ψ score of one or more genes selected from: Nek6, Pphln1, Pdgfc, Pomt1, Sorbs1, Ssfa2, Rps6kb2, Irak1, Cpeb4, Calu, Gtpbp10, Pard3, Ranbp3, Prx, Rad18, 2810474019Rik, Mtdh, Sema6d, Ar16, Tbp, Slx4, Abi1, Osbpl1a, Pex7, Camklg, Idnk, Zfp963, Firre, Grik1, Ktn1, Nmbr, Sec1412, Gm21992, Mpnd, Elavl2, Mtmr1, Six3os1, Tenm4, Zyx, Camsap3, Brat1, Hdac5, Fmnl3, Pisd, Atp11c,Lair1, Ccnc, Nnat, Fam111a, Mettl4, D130020L05Rik, Etfrf1, Chd2, Ikzf4, Phkb, A730017C20Rik, Tnrc6a, Itga3, Gprasp1, Ptprf, Cd200, Dync1i2, Huwe1, Aplp2, Ctbs, Mink1, Dpy19l4, Ccdc85a, Asph, Nr3c2, Kif2a, Dennd5a, Pip5k1c, Plxna3, Arhgef9, Iqsec2, Fyttd1, Smyd4, Tmem234, Aif1l, Adgrl3, 0610037L13Rik, 1600014C10Rik, 1700001L05Rik, 3010001F23Rik, 6430573F11Rik, 9030617003Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcrl, Camkk2, Ccdc43, Ccdc66, Cd180, Cdkl4, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Deaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4gl, Em14, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Galns, Glt8d1, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcnt1, Klc4, Las1l, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf412, Morn1, Mpv1712, Mthfsl, Myo1e, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pedh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfia1, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassf2, Rbm10, Rbm12, Rbm39, Rbms3, Rcbtb2, Retreg3, Rffl, Rhobtb1, Rhoj, Ripor2, Rpain, Rpgr, Rps24, Rps6kal, Rtel1, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1, Snap23, Snrpa1, Snx21, Spint2, Spire1, Ssr1, St3gal3, St3ga16, Stard13, Stoml2, Stx2, Stx4a, Stxbp5l, Sugp2, Synpo, Taf12, Tcte3, Terf1, Tex30, Thrap3, Thyn1, Ijap1, Tle3, Tlr4, Tlr7, Tmem134, Tmem209, Tmem25, Tmem44, Tpd52, Tpd52l2, Trp53i13, Trpcl, Ttc17, Ttc21b, Ttll5, Tysndl, Ube2cbp, Ubn2, Ubxn7, Uhrf1bp1, Unc13b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Spp1, Plek, Col5a3, S1pr3, Anxa2, Mt1, Sbno2, Csf1, Ch25h, Mt2, Serpine1, Vim, Co116a1, Neat1, Cd109, Dysf, C3ar1, Pros1, Tnfrsf1a, Tgm1, Socs3, Rplp0, Osmr, Cebpd, Tspan7, Srxn1, Capg, Ahnak, Fgf13, S100a10, Arpc1b, Slc6a1, Lgals3, Bcl3, Plin2, Csf2rb2, Clic1, Gfap, Aspg, Emp1, and/or Msr1.

In some embodiments, the method comprises detecting (e.g., quantifying) in a second biological sample obtained from the subject at a second point in time a second Ψ score of one or more genes selected from: Nek6, Pphln1, Pdgfc, Pomt1,Sorbs1, Ssfa2, Rps6kb2, Irak1, Cpeb4, Calu, Gtpbp10, Pard3, Ranbp3, Prx, Rad18, 2810474019Rik, Mtdh, Sema6d, Ar16, Tbp, Slx4, Abi1, Osbpl1a, Pex7, Camklg, Idnk, Zfp963, Firre, Grik1, Ktnl, Nmbr, Sec1412, Gm21992, Mpnd, Elavl2, Mtmrl, Six3os1, Tenm4, Zyx, Camsap3, Brat1, Hdac5, Fmnl3, Pisd, Atp11c, Lair1, Ccnc, Nnat, Fam111a, Mettl4, D130020L05Rik, Etfrf1, Chd2, Ikzf4, Phkb, A730017C20Rik, Tnrc6a, Itga3, Gprasp1, Ptprf, Cd200, Dyncli2, Huwe1, Aplp2, Ctbs, Mink1, Dpy19l4, Ccdc85a, Asph, Nr3c2, Kif2a, Dennd5a, Pip5k1c, Plxna3, Arhgef9, Iqsec2, Fyttd1, Smyd4, Tmem234, Aif1l, Adgrl3, 0610037L13Rik, 1600014C10Rik, 1700001L05Rik, 3010001F23Rik, 6430573F11Rik, 9030617003Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacnalc, Cadm1, Cadps, Calcrl, Camkk2, Ccdc43, Ccdc66, Cd180, Cdk14, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Cspp1, Csrnp3, Ctnnd1, Cwc22, Dab2, Deaf17, Dclk1, Dgkb, Dhrs3, Disc1, Dkc1, Dlg1, Dlg3, Dlgap4, Dnm2, Donson, Dst, Dtnb, Eefld, Efcab6, Eif2ak4, Eif4g1, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Galns, Glt8d1, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il1Sra, Inf2, Inpp4a, Inppl1, Ints10, Invs, Itgam, Itgb5, Itpr2, Itsn1, Itsn2, Kansl1, Kantr, Kars, Kcnk2, Kcnt1, Klc4, Las1l, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metapld, Mid1, Mknk1, Morf4l2, Morn1, Mpv17l2, Mthfsl, Myo1e, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pcdh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfial, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassj2, Rbm10, Rbm12, Rbm39, Rbms3, Rcbtb2, Retreg3, Rffl, Rhobtb1, Rhoj, Ripor2, Rpain, Rpgr, Rps24, Rps6ka1, Rtel1, Rtn4, Ruben, Runx1t1, Sbf1, Scpep1, Scrn3, Sema4d, Sema6a, Serpinb6a, Serpinh1, Setd3, Sez61, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1, Snap23, Snrpal, Snx21, Spint2, Spire1, Ssr1, St3gal3, St3gal6, Stard13, Stoml2, Stx2, Stx4a, StxbpSl, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd5212, Trp53i13, Trpc1, Ttc17, Ttc21b, Ttll5, Tysnd1, Ube2cbp, Ubn2, Ubxn7, Uhrf1bp1, Unc13b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Finc, Msn, Serpina3n, Cd44, Stat3, Igfbp3, Cd63, Ucp2, Spp1, Plek, Col5a3, S1pr3, Anxa2, Mt1, Sbno2, Csf1, Ch25h, Mt2, Serpine1, Vim, Col16a1, Neat1, Cd109, Dysf, C3ar1, Pros1, Tnfrsfla, Tgm1, Socs3, RplpO, Osmr, Cebpd, Tspan7, Srxn1, Capg, Ahnak, Fgf13, S100a10, Arpc1b, Slc6a1, Lgals3, Bcl3, Plin2, Csf2rb2, Clic1, Gfap, Aspg, Empl, and/or Msr1.

Finally, in some embodiments, the method comprises diagnosing the subject as having progressed from a pre-symptomatic ALS/FTD disease state to an acute ALS/FTD disease state if the value of the second Ψ score is increased or decreased relative to the first Ψ score (e.g., if the delta psi (ΔΨ) score is not zero (0)).

In some embodiments, a biological sample (e.g., blood, serum, or tissue) is obtained from a subject. In some embodiments, the biological sample (e.g., blood, serum, or tissue) is obtained from a subject that is genopositive for a C9orf72 expansion repeat (e.g., is C9-positive). As used herein, “genopositive” refers to a subject who has tested positive for the presence of certain markers associated with the presence of a C9orf72 expansion repeat (e.g., RAN proteins).

A subject may be determined to be genopositive, for example, based on the presence of one or more mutations in a gene associated with ALS/FTD, such as, e.g., C90rf72, SOD1, etc., and/or based on a level of one or more di-amino acid repeat-containing proteins in a biological sample taken from the subject.

Thus, in some embodiments the subject is identified as genopositive based upon the detection of a heterozygous C9orf72 pathogenic GGGGCC (G₄C₂) hexanucleotide repeat expansion using molecular genetic testing techniques (e.g., genotyping, sequencing, amplifying/hybridizing, etc.). In some embodiments, the repeat expansion is greater than 30 repeats (e.g., 31, 32, 33, 34, 35, 50, 75, 100, or more repeats) (SEQ ID NO: 3). In some embodiments, the subject is identified as genopositive based upon the method of diagnosis described in U.S. Pat. No. 10,295,547, incorporated by reference herein in its entirety.

In some embodiments, the subject is identified as genopositive based on a level of one or more di-amino acid repeat-containing proteins in a sample (e.g., blood, serum, or tissue) obtained from a subject. In some embodiments, the di-amino acid repeat-containing proteins are selected from the group consisting of: poly-(Gly-Ala), poly-(Gly-Pro), poly-(Gly-Arg), poly-(Pro-Ala), poly-(Pro-Arg), Met... poly-(Pro-Arg) or Met... poly-(Gly-Pro). In some embodiments, a level of the one or more di-amino acid-repeat-containing proteins that is elevated in the sample compared to a control level indicates that the subject is genopositive (e.g., a “positive sample”). In some embodiments, a positive sample comprises a number of di-amino acid repeat-containing proteins present in the sample, for example 30, 50, 100, or 1,000 di-amino acid repeat-containing proteins. Each di-amino acid repeat-containing protein comprises a repeat amino acid sequence, which contains a di-amino acid repeat unit of the formula (YZ)ₓ, where X can be from 2-10,000, 5-10,000, 2-5,000, 5-5,000, 2-1000, 5-1000, 5-500, 5-300, 5-200, 10-500, 10-300, or 10-200.

In some embodiments, the two biological samples are obtained from the same subject at two different points in time (e.g., a first sample is obtained at a first point in time, and a second sample is obtained at a second point in time). In some embodiments, the second biological sample is a sample taken from the same subject 1 hour later, 1 day later, 2 days later, 3 days later, 4 days later, 5 days later, 6 days later, 1 week later, 2 weeks later, 3 weeks later, 1 month later, 2 months later, 3 months later, 6 months later, 1 year later, 2 years later, 3 years later, 4 years later, 5 years later, 10 years later, 20 years later, etc. than the first biological sample.

In some embodiments, the method comprises diagnosing the subject as having progressed from a pre-symptomatic ALS/FTD disease state to an acute ALS/FTD disease state if the value of the second Ψ score is increased or decreased relative to the first Ψ score (e.g., if the delta psi (ΔΨ) score is not zero (0)). As used herein, delta psi (ΔΨ) score is used to refer to the calculation of two Ψ scores for a single gene of interest (e.g., at different points in time in the same subject, or at the same or different points in time in two different subjects). The difference between the two calculated Ψ scores is the ΔΨ score.

It will be understood that, because a Ψ score may be any value between 0 and 1, as described herein, a ΔΨ score (that is, the difference between two calculated Ψ scores) may be any value between 0 and 1 (e.g., 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0, or any value included therein such as, e.g., 0.001, 0.0001, 0.0001, etc.) or any value between 0 and -1 (e.g., 0, -0.01, -0.02, -0.03, -0.04, -0.05, -0.06, -0.07, -0.08, -0.09, -0.10, -0.11, -0.12, -0.13, -0.14, -0.15, -0.16, -0.17, -0.18, -0.19, -0.20, -0.21, -0.22, -0.23, -0.24, -0.25, -0.26, -0.27, -0.28, -0.29, -0.30, -0.31, -0.32, -0.33, -0.34, -0.35, -0.36, -0.37, -0.38, -0.39, -0.40, -0.41, -0.42, -0.43, -0.44, -0.45, -0.46, -0.47, -0.48, -0.49, -0.50, -0.51, -0.52, -0.53, -0.54, -0.55, -0.56, -0.57, -0.58, -0.59, -0.60, -0.61, -0.62, -0.63, -0.64, -0.65, -0.66, -0.67, -0.68, -0.69, -0.70, -0.71, -0.72, -0.73, -0.74, -0.75, -0.76, -0.77, -0.78, -0.79, -0.80, -0.81, -0.82, -0.83, -0.84, -0.85, -0.86, -0.87, -0.88, -0.89, -0.90, -0.91, -0.92, -0.93, -0.94, -0.95, -0.96, -0.97, -0.98, -0.99, or -1.0, or any value included therein such as, e.g., -0.001, -0.0001, -0.0001, etc.). In some embodiments, a ΔΨ score may be expressed as an absolute value, where, for example, the absolute value of -0.1 is 0.1.

Thus, in some embodiments, the subject is diagnosed as having progressed from a pre-symptomatic ALS/FTD disease state to an acute ALS/FTD disease state if the ΔΨ score is not zero (0) (e.g., if value of the second Ψ score is increased or decreased relative to the first Ψ score). In some embodiments, certain isoforms of the certain genes as described herein may undergo increased alternative splicing as the ALS/FTD state progresses. In these embodiments, the Ψ score of the second sample would be increased relative to the Ψ score of the first sample and the ΔΨ score would be positive (e.g., greater than 0). In other embodiments, certain isoforms of the certain genes as described herein may undergo decreased alternative splicing as the ALS/FTD state progresses. In these embodiments, the Ψ score of the second sample would be decreased relative to the Ψ score of the first sample and the ΔΨ score would be negative (e.g., less than 0). In either of these embodiments (e.g., where alternative splicing is increased or decreased as the ALS/FTD state progresses), the absolute value of the ΔΨ score would be not zero (0), for example 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0, or any value included therein such as, e.g., 0.001, 0.0001, 0.0001, etc.

### Methods of Treatment

In some embodiments, the subject characterized as having acute or pre-symptomatic ALS/FTD, or as having progressed from a pre-symptomatic ALS/FTD disease state to an acute ALS/FTD disease state, based on use of the methods described herein is treated. As used herein, “treat” or “treatment” refers to (a) preventing or delaying the onset of ALS or FTD; (b) reducing the severity of ALS or FTD; (c) reducing or preventing development of symptoms characteristic of ALS or FTD; (d) preventing worsening of symptoms characteristic of ALS or FTD; and/or (e) reducing or preventing recurrence of ALS or FTD symptoms in subjects that were previously symptomatic for ALS or FTD.

In some embodiments, the subject characterized as having acute or pre-symptomatic ALS/FTD, or as having progressed from a pre-symptomatic ALS/FTD disease state to an acute ALS/FTD disease state, based on use of the methods described herein is treated by the administration of a therapeutic agent. In some embodiments, the therapeutic agent targets a gene, or multiple genes, which were identified to undergo alternative splicing according to the methods of the present disclosure. In some embodiments, the therapeutic agent targets a DNA repair pathway gene or gene product, which may, for example, relate to a gene, or multiple genes, which were identified to undergo alternative splicing according to the methods of the present disclosure. In some embodiments, administration of a therapeutic agent which targets a gene, or multiple genes, which were identified to undergo alternative splicing according to the methods of the present disclosure to a subject results in the treatment of ALS/FTD.

In some embodiments, the therapeutic agent used to treat the subject having ALS/FTD is a peptide, protein, nucleic acid, or small molecule.

In some embodiments, the therapeutic peptide is a peptide vaccine that targets a RAN protein. In some embodiments, a therapeutic protein is an anti-RAN protein vaccine. In some embodiments, an anti-RAN protein vaccine comprises a peptide antigen comprising an amino acid repeat sequence selected from poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine-Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 4); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 5); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 6); poly(LLPSPSRC) (SEQ ID NO: 7); poly(YSPLPPGV) (SEQ ID NO: 8); poly(HREGEGSK) (SEQ ID NO: 9); poly(TGRERGVN) (SEQ ID NO: 10); poly(PGGRGE) (SEQ ID NO: 11); poly(GRQRGVNT) (SEQ ID NO: 12); and poly(GSKHREAE) (SEQ ID NO: 13).

In some embodiments, the therapeutic protein is a protein that modifies eukaryotic initiation factor 2 (eIF2), eukaryotic initiation factor 3 (eIF3), protein kinase R (PKR), p62, LC3 I subunit, LC3 II subunit, or Toll-like receptor 3 (TLR3). In some embodiments, the therapeutic protein is a protein that is a dominant-negative variant of protein kinase R (PKR) or a dominant-negative variant of TLR3 protein. In some embodiments, a dominant-negative variant comprises a mutation at amino acid position 296. In some embodiments, the mutation is K296R.

In some embodiments, the therapeutic is a small molecule, such as tetrabenazine, haloperidol, chlorpromazine, risperidone, quetiapine, amantadine, levetiracetam, clonazepam, citalopram, fluoxetine, sertraline, olanzapine, alproate, carbamazepine, lamotrigine, cysteamine, PBT2, PDE10A inhibitor, pridopidine, and laquinimod. In some embodiments, a small molecule is a modifier of eukaryotic initiation factor 2 (eIF2), eukaryotic initiation factor 3 (eIF3), protein kinase R (PKR), p62 (sequestome-1 or ubiquitin binding protein), LC3 (microtubule associated protein 1 light chain 3) I subunit, LC3 II subunit, or Toll-like receptor 3 (TLR3). In some embodiments, a small molecule is metformin or a pharmaceutically acceptable salt, co-crystal, tautomer, stereoisomer, solvate, hydrate, polymorph, isotopically enriched derivative, or prodrug thereof. In some embodiments, a small molecule is buformin, phenformin, metformin, or a derivative or functional analogue thereof. In some embodiments, a small molecule is an inhibitor of PKR such as TARBP2.

In some embodiments, the therapeutic nucleic acid is an interfering RNA, such as, for example, a dsRNA, siRNA, miRNA, amiRNA, ASO, aptamer, etc. In some embodiments, an interfering nucleic acid is a dsRNA, siRNA, shRNA, miRNA, artificial miRNA (amiRNA), or antisense oligonucleotide (ASO). In some embodiments, an interfering nucleic acid modifies expression of eukaryotic initiation factor 2 (eIF2), eukaryotic initiation factor 3 (eIF3), protein kinase R (PKR), p62, LC3 I subunit, LC3 II subunit, or Toll- like receptor 3 (TLR3). In some embodiments, an interfering nucleic acid modifies expression of eIF2A or eIF2a. In some embodiments, an interfering nucleic acid inhibits expression of one or more eIF3 subunits selected from the group consisting of eIF3a, eIF3b, eIF3c, eIF3d, eIF3e, eIF3f, eIF3g, eIF3h, eIF3i, eIF3j, eIF3k, eIF31, and eIF3m. In some embodiments, an interfering nucleic acid inhibits expression of protein kinase R (PKR).

In some embodiments, the therapeutic protein is an antibody. In some embodiments, an antibody targets eukaryotic initiation factor 2 (eIF2), eukaryotic initiation factor 3 (eIF3), protein kinase R (PKR), p62, LC3 I subunit, LC3 II subunit, or Toll-like receptor 3 (TLR3). Such antibodies are known in the art (see, e.g., Duffy, et al., Cell Immunol. (2007) Aug;248(2):103-14. PubMed PMID: 18048020). In some embodiments, the antibody is an anti-RAN protein antibody. In some embodiments, the anti-RAN protein antibody binds to a di-amino acid repeat region of a RAN protein. In some embodiments, an anti-RAN protein antibody targets any one or more of poly(Proline-Arginine) [poly(PR)]; poly(Glycine-Arginine) [poly(GR)]; poly(Serine) [polySer]; poly(Cysteine-Proline) [poly(CP)]; poly(Glycine-Proline) [(poly(GP)]; poly(Glycine) [poly(G)]; poly(Alanine) [polyAla]; poly(Glycine-Alanine) [poly(GA)]; poly(Glycine-Aspartate) [poly(GD)]; poly(Glycine-Glutamate) [poly(GE)]; poly(Glycine-Glutamine) [poly(GQ)]; poly(Glycine- Threonine) [poly(GT)]; poly(Leucine) [polyLeu]; poly(Leucine-Proline) [poly(LP)]; poly(Leucine-Proline-Alanine-Cysteine) [poly(LPAC)] (SEQ ID NO: 4); poly(Leucine-Serine) [poly(LS)]; poly(Proline) [poly(P)]; poly(Proline-Alanine) [poly(PA)]; poly(Glutamine-Alanine-Glycine-Arginine) [poly(QAGR)] (SEQ ID NO: 5); poly(Arginine-Glutamate) [poly(RE)]; poly(Serine-Proline) [poly(SP)], poly(Valine-Proline) [poly(VP)], poly(phenylalanine-proline) [poly(FP)], poly(glycine-lysine) [poly(GK)], poly(FTPLSLPV) (SEQ ID NO: 6); poly(LLPSPSRC) (SEQ ID NO: 7); poly(YSPLPPGV) (SEQ ID NO: 8); poly(HREGEGSK) (SEQ ID NO: 9); poly(TGRERGVN) (SEQ ID NO: 10); poly(PGGRGE) (SEQ ID NO: 11); poly(GRQRGVNT) (SEQ ID NO: 12); and poly(GSKHREAE) (SEQ ID NO: 13). In some embodiments, an anti-RAN protein antibody specifically binds to the poly-amino acid repeat of the RAN protein. In some embodiments, an anti-RAN protein antibody specifically binds to the C-terminus of the RAN protein. In some embodiments, an anti-RAN protein antibody is a monoclonal antibody. In some embodiments, an anti-RAN protein antibody is a polyclonal antibody. In some embodiments, anti-RAN protein antibodies are generated with binding activity to newly identified RAN proteins occurring in the RAN protein-associated neurological disease, which are predicted by the sequences of the novel enriched repeat expansion mutations.

Those skilled in the art will understand how to make antibodies binding to the enumerated protein targets and to screen for the desired modulation of the functions of the target proteins. Briefly, an anti-RAN antibody can be a polyclonal antibody or a monoclonal antibody. Typically, polyclonal antibodies are produced by inoculation of a suitable mammal, such as a mouse, rabbit, or goat. Larger mammals are often preferred as the amount of serum that can be collected is greater. Typically, an antigen (e.g., an antigen comprising a poly-Ser repeat region) is injected into the mammal. This induces the B-lymphocytes to produce IgG immunoglobulins specific for the antigen. This polyclonal IgG is purified from the mammal’s serum. Monoclonal antibodies are generally produced by a single cell line (e.g., a hybridoma cell line). In some embodiments, an anti-RAN antibody is purified (e.g., isolated from serum).

Numerous methods may be used for obtaining anti-RAN antibodies. For example, antibodies can be produced using recombinant DNA methods. Monoclonal antibodies may also be produced by generation of hybridomas (see, e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (e.g., OCTET or BIACORE) analysis, to identify one or more hybridomas that produce an antibody that specifically binds with a specified antigen. Any form of the specified antigen (e.g., a RAN protein) may be used as the immunogen, e.g., recombinant antigen, naturally occurring forms, or any variants or fragments thereof. One exemplary method of making antibodies includes screening protein expression libraries that express antibodies or fragments thereof (e.g., scFv), e.g., phage or ribosome display libraries. Phage display is described, for example, in Ladner, et al., U.S. Pat. No. 5,223,409; Smith (1985) Science 228:1315-1317; Clackson, et al. (1991) Nature, 352: 624-628; Marks, et al. (1991) J. Mol. Biol., 222: 581-597; WO92/18619; WO 91/17271; WO 92/20791; WO 92/15679; WO 93/01288; WO 92/01047; WO 92/09690; and WO 90/02809.

In addition to the use of display libraries, the specified antigen (e.g., one or more RAN proteins, such as poly-Ser) can be used to immunize a non-human animal, e.g., a rodent, e.g., a mouse, hamster, or rat. In one embodiment, the non-human animal is a mouse.

In another embodiment, a monoclonal antibody is obtained from the non-human animal, and then modified, e.g., made chimeric, using recombinant DNA techniques known in the art. A variety of approaches for making chimeric antibodies have been described. See, e.g., Morrison, et al., Proc. Natl. Acad. Sci. U.S.A. 81:6851, 1985; Takeda, et al., Nature 314:452, 1985, Cabilly, et al., U.S. Pat. No. 4,816,567; Boss, et al., U.S. Pat. No. 4,816,397; Tanaguchi, et al., European Patent Publication EP171496; European Patent Publication 0173494; and United Kingdom Patent GB 2177096B.

Antibodies can also be humanized by methods known in the art. For example, monoclonal antibodies with a desired binding specificity can be commercially humanized (Scotgene, Scotland; and Oxford Molecular, Palo Alto, Calif.). Fully humanized antibodies, such as those expressed in transgenic animals, are within the scope of the invention (see, e.g., Green, et al. (1994) Nature Genetics 7, 13; and U.S. Pat. Nos. 5,545,806 and 5,569,825).

For additional antibody production techniques, see Antibodies: A Laboratory Manual, Second Edition. Edited by Edward A. Greenfield, Dana-Farber Cancer Institute, ©2014. The present disclosure is not necessarily limited to any particular source, method of production, or other special characteristics of an antibody.

In some embodiments, treatment comprises administering an effective amount of a known ALS therapeutic agent, such as Riluzole (Rilutek, Sanofi-Aventis), to a subject identified as having ALS. In some embodiments, treatment comprises administering an effective amount of a known FTD therapeutic agent, such as trazodone (Desyrel, Oleptro) or a selective serotonin reuptake inhibitor (SSRI), to a subject identified as having FTD. In some embodiments, treatment comprises administering an effective amount of a therapeutic agent, such as baclofen, diazepam, phenytoin, trihexyphenidyl and/or amitriptyline, which reduces one or more symptoms of ALS and/or FTD in a subject identified as having ALS and/or FTD.

In some embodiments, a therapeutic agent (e.g., a nucleic acid encoding a therapeutic protein, interfering nucleic acid, etc.) is delivered to the subject by a vector. In some embodiments, a vector is a viral vector. In some embodiments, a viral vector is a recombinant adeno-associated virus (rAAV) vector or a lentivirus vector.

In some embodiments, treatment comprises one or more of physical therapy, occupational therapy, or speech therapy. In some embodiments, treatment comprises a method for decreasing or stabilizing di-amino acid-repeat-containing protein levels in the blood of the subject, such as bone marrow transplantation or plasmapheresis. In some embodiments, treatment comprises any combination of the above-mentioned treatments or any other treatments described herein.

An effective amount is a dosage of a therapeutic agent sufficient to provide a medically desirable result, such as treatment of ALS or FTD. The effective amount will vary with the age and physical condition of the subject being treated, the severity of ALS or FTD in the subject, the duration of the treatment, the nature of any concurrent therapy, the specific route of administration and the like factors within the knowledge and expertise of the health practitioner. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. Generally, an amount of active agent is included in each dosage form to provide from about 0.1 to about 250 mg/kg, and preferably from about 0.1 to about 100 mg/kg. One of ordinary skill in the art would be able to determine empirically an appropriate therapeutically effective amount. Dosage can be determined by the skilled artisan. In certain embodiments, a dose (e.g., a single dose, or any dose of multiple doses) described herein includes independently between 0.1 µg and 1 µg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a therapeutic agent described herein. In certain embodiments, a dose described herein includes independently between 1 mg and 3 mg, inclusive, of a therapeutic agent described herein. In certain embodiments, a dose described herein includes independently between 3 mg and 10 mg, inclusive, of a therapeutic agent described herein. In certain embodiments, a dose described herein includes independently between 10 mg and 30 mg, inclusive, of a therapeutic agent described herein. In certain embodiments, a dose described herein includes independently between 30 mg and 100 mg, inclusive, of a therapeutic agent described herein.

Administration of a treatment may be accomplished by any method known in the art (see, e.g., Harrison’s Principle of Internal Medicine, McGraw Hill Inc.). Administration may be local or systemic. Compositions for different routes of administration are well known in the art (see, e.g., Remington’s Pharmaceutical Sciences by E. W. Martin). A therapeutic agent can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject. Several types of devices are regularly used for administration by inhalation. These types of devices include metered dose inhalers (MDI), breath-actuated MDI, dry powder inhaler (DPI), spacer/holding chambers in combination with MDI, and nebulizers.

In some embodiments, a therapeutic agent is administered to the central nervous system (CNS) of a subject in need thereof. As used herein, the “central nervous system (CNS)” refers to all cells and tissues of the brain and spinal cord of a subject, including, but not limited to, neuronal cells, glial cells, astrocytes, cerebrospinal fluid, etc. Modalities of administering a therapeutic agent to the CNS of a subject include direct injection into the brain (e.g., intracerebral injection, intraventricular injection, intraparenchymal injection, etc.), direct injection into the spinal cord of a subject (e.g., intrathecal injection, lumbar injection, etc.), or any combination thereof.

In some embodiments, a treatment as described by the disclosure is systemically administered to a subject, for example by intravenous injection. Systemically administered therapeutic molecules can be modified, in some embodiments, in order to improve delivery of the molecules to the CNS of a subject. Examples of modifications that improve CNS delivery of therapeutic molecules include, but are not limited to, co-administration or conjugation to blood brain barrier-targeting agents (e.g., transferrin, melanotransferrin, low-density lipoprotein (LDL), angiopeps, RVG peptide, etc., as disclosed by Georgieva, et al., Pharmaceuticals 6(4): 557-583 (2014)), coadministration with BBB disrupting agents (e.g., bradykinins), and physical disruption of the BBB prior to administration (e.g., by MRI-Guided Focused Ultrasound), etc.

Other aspects of the disclosure relate to methods for monitoring responsiveness to a therapeutic agent (e.g., a treatment for ALS/FTD) in a subject having ALS or FTD or suspected of having ALS or FTD. Without wishing to be bound by any particular theory, detection (e.g., quantification) of certain isoforms of one or more genes as described herein (e.g., in Table 4) in a biological sample obtained from a subject can be used to determine the effectiveness of a therapeutic agent (e.g., a treatment for ALS/FTD) in the subject from which the samples are obtained. For example, measuring a psi score which is closer to zero (0) (e.g., for positive or negative psi scores, closer to 0) psi score of one or more isoforms of interest in a subject after administration of a therapeutic agent for the treatment of ALS/FTD (e.g., relative to the psi score(s) measured for the same isoform(s) in the subject prior to the administration, or in a control subject) is indicative of the therapeutic agent effectively treating the subject for ALS/FTD. Measuring a psi score which is farther from zero (0) (e.g., for positive psi scores, closer to 1; for negative psi scores, closer to -1) psi score of one or more isoforms of interest in a subject after administration of a therapeutic agent for the treatment of ALS/FTD (e.g., relative to the psi score(s) measured for the same isoform(s) in the subject prior to the administration, or in a control subject) is indicative of the therapeutic agent not effectively treating the subject for ALS/FTD.

A psi score which is quantified for a certain isoform(s) of one or more genes in a subject after administration of a therapeutic agent (e.g., a treatment for ALS/FTD) may be compared to the psi score(s) measured for the same isoform(s) in the subject prior to the administration, in some embodiments, of may be compared to a control psi score for the same isoform(s), in some embodiments. A control psi score may be obtained from a healthy subject, for example a subject who does not have, and is not suspected of having, ALS/FTD. Healthy subjects are described elsewhere herein. As used herein, a control psi score may also refer to a psi score which is quantified for a certain isoform(s) of one or more genes in a subject prior to the onset of symptoms which are known to be associated with ALS/FTD and/or prior to the diagnosis of ALS/FTD via genetic testing or other means, as described herein (e.g., when the subject is considered to be a healthy subject, as defined herein).

In some embodiments, a method of monitoring responsiveness to a treatment in a subject having ALS or FTD or suspected of having ALS or FTD comprises: (i) obtaining a biological sample from a subject who has not been administered a treatment for ALS/FTD, (ii) calculating one or more psi scores for certain isoforms of certain genes as described herein (e.g., in Table 4) in the first biological sample, (iii) administering a therapeutic agent (e.g., a treatment for ALS/FTD) to the subject, (iv) obtaining a second biological sample from the subject, (v) calculating one or more psi scores for the same certain isoforms of certain genes as were calculated in (ii) in the second biological sample, and (vi) continuing to administer the treatment if the psi score(s) of the certain isoforms are closer to zero (0) (e.g., for positive or negative psi scores, closer to 0) in the calculation of step (v), relative to step (ii).In some embodiments, a method of monitoring responsiveness to a treatment in a subject having ALS or FTD or suspected of having ALS or FTD comprises: (i) obtaining a first biological sample from a subject who has not been administered a treatment for ALS/FTD, (ii) calculating one or more psi scores for certain isoforms of certain genes as described herein (e.g., in Table 4) in the first biological sample, (iii) administering a therapeutic agent (e.g., a treatment for ALS/FTD) to the subject, (iv) obtaining a second biological sample from the subject, (v) calculating one or more psi scores for the same certain isoforms of certain genes as were calculated in (ii) in the second biological sample, and (vi) not continuing to administer or altering the treatment if the psi score(s) of the certain isoforms are farther from zero (0) (e.g., for positive psi scores, closer to 1; for negative psi scores, closer to -1) in the calculation of step (v), relative to step (ii).

The time between which a first biological sample and a second biological sample are obtained may vary. In some embodiments, a first biological sample is obtained between 1 week and 1 minute prior to administration of a therapeutic agent (e.g., the first administration of a therapeutic agent). In some embodiments, a first biological sample is obtained between 1 day (e.g., 24 hours) and 1 minute prior to administration of a therapeutic agent (e.g., the first administration of a therapeutic agent). In some embodiments, a second biological sample is obtained from the subject between 1 minute and six months after administration of a therapeutic agent (e.g., the first administration of a therapeutic agent). In some embodiments, a second biological sample is obtained from the subject between 1 day and 1 week after administration of a therapeutic agent (e.g., the first administration of a therapeutic agent). In some embodiments, a second biological sample is obtained from the subject between 1 day and 1 week after administration of a therapeutic agent (e.g., the most recent or last administration of a therapeutic agent).

In some embodiments, a second biological sample may be collected about 1 hour, 5 hours, 10 hours, 24 hours (e.g., 1 day), 48 hours (e.g., 2 days), 120 hours (e.g., 5 days), 30 days, 45 days, or six months after administration of the therapeutic agent. In some embodiments, several biological samples (e.g., 2, 3, 4, 5, 6, 7, 8, 9, 10, or more biological samples) are obtained from the subject, for example over a specified timeframe (e.g., during a therapeutic course).

### EXAMPLES

The intronic C9orf72 GGGGCC (G₄C₂) hexanucleotide microsatellite repeat expansion mutation is the most commonly known genetic cause of amyotrophic lateral sclerosis (ALS) and frontotemporal dementia (FTD). There is remarkable clinical heterogeneity among C9orf72 expansion carriers, with clinical presentation ranging from the muscle wasting disease of ALS found in some patients to the disinhibition and cognitive deficits characteristic of FTD in others. While some expansion carriers remain asymptomatic into their 90s, the estimates of the rate of reduced penetrance are not yet clear due to ascertainment bias. The contributions of repeat length and somatic repeat instability, which are known to contribute to other expansion disorders, may contribute to incomplete penetrance, differences in age-of-onset and the wide-ranging clinical effects of the C9orf72 expansion mutation. However, ascertainment bias, technical difficulties in measuring repeat length, and somatic instability have made understanding the effects of repeat length as a modifier of C9orf72 ALS/FTD difficult.

Molecular mechanisms that have been proposed for C9orf72 ALS/FTD include C9orf72 protein loss-of-function, RNA gain-of-function, and repeat associated non-ATG (RAN) protein toxicity. A number of mouse models have been developed to better understand the relative contributions of loss of function and/or gain of function mechanisms in disease. C9orf72 knockout mice develop peripheral immune phenotypes, but do not develop ALS/FTD related phenotypes, making it unlikely that C9orf72 loss of function alone is a major driver of disease. In contrast, Drosophila and mouse models overexpressing specific RAN proteins develop neurodegenerative and motor phenotypes, indicating RAN proteins can be toxic and may play a role in disease.

Similarly, RNA gain of function effects may cause RNA processing abnormalities that contribute to disease. Studies on C9 iPSC-derived neurons (iPSNs) and C9-ALS autopsy tissue have reported transcriptomic and alternative splicing abnormalities compared to controls. Additionally, a number of RNA binding proteins (RNA-BPs) that interact with short stretches of G₄C₂ repeats have been identified through unbiased interactome screens including Pur-α, ADARB2, hnRNPH, hnRNPA1, hnRNPA2/B1, ALYREF, nucleolin and RanGAP1, and confirmed in overexpression cell culture systems. Crosslinking immunoprecipitation (CLIP) analyses using autopsy material from the frontal cortex of C9-ALS patients show that hnRNPH-H binds to G₄C₂ repeats and co-localizes with RNA foci in patient cells, although this was demonstrated indirectly using an antibody to G quadruplexes. Since there is little consensus on which RNA binding proteins are sequestered by the G₄C₂ repeats, the role of RNA gain-of-function mechanisms in C9orf72 ALS/FTD remains unclear.

Bacterial artificial chromosome (BAC) transgenic mouse models have been generated to understand C9-ALS/FTD disease mechanisms. All four of the C9-BAC models develop the molecular features of the disease, including sense and antisense RNA foci and RAN protein aggregates, although the relative levels of sense and antisense transcripts and RAN proteins have not been directly compared. Three of these models, which were developed on B6 genetic backgrounds, showed no, or subtle, hippocampal degeneration and social behavioral deficits, but not the typical features of ALS/FTD. In contrast, BAC transgenic mice developed on the FVB background showed that female mice from several independent lines develop both the molecular and behavioral features of ALS/FTD, including behavioral abnormalities, paralysis, motor neuron loss, and decreased survival. Here, it is shown that these FVB-C9 mice develop ALS/FTD phenotypes independent of integration effects and C9orf72 protein levels. Using the most penetrant C9-500 line, an allelic series of mice containing 800, 500 or 50 repeats was generated, and it is demonstrated that longer repeat tracts increase disease penetrance and decrease age-of-onset. Additionally, it is shown that transcriptomic profiles are distinct at different disease stages in C9-BAC mice, and that alternative splicing abnormalities are prevalent prior to onset of overt symptoms, thus suggesting their potential role in disease and their utility as early biomarkers of ALS/FTD.

### Example 1: Materials and Methods

### Mate Pair Preparation and Whole Genome Sequencing

Input DNA was received and quantified using a fluorometric-based method specific for double stranded DNA. The quality of the genomic DNA was evaluated using capillary electrophoresis-based technology (Fragment Analyzer, AATI). Four micrograms of good quality DNA were processed into Illumina-compatible libraries using Nextera Mate Pair Sample Preparation Kit (Illumina) following the Gel-Plus procedure with following modifications. The fragmented DNA was size-selected for a target range of 5-8 kb fragments using an automated DNA size-selection technology (BluePippin (Sage Science)), and the adapter-ligated mate pair fragments were enriched using 10 amplification cycles. Final libraries were quantified using the KAPA Library Quantification Kit (KAPA Biosystem), Qubit Fluorometer (Life Technologies), and 2100 BioAnalyzer (Agilent), and were sequenced on an Illumina HiSeq2500 sequencer using 2 × 125 bp cycles.

### Bioinformatics Analysis for Transgene Integration

All read data was processed prior to alignment. Processing included (1) ab initio duplicate removal using in-house scripts; (2) adapter identification/clipping and low-quality end trimming (Q=20) using Cutadapt 1.8.1; (3) phiX decontamination by mapping with GEM mapper against the phiX reference (acc. NC_001422.1). Read pairs were further screened for the presence of the Nextera ligation adapter, which was also trimmed. After determination of the library insert size and mate orientation by mapping a subset of the reads against the mouse reference genome (mm10) with the GEM mapper, downstream analysis was considered for the entire set of processed mate-pairs, given that the overall percentage of reads mapping in forward-reverse orientation (representing paired-end contamination) was low (-1%). Processed read data was then mapped, using BWA-MEM, against a combined index that included, apart from the full mouse reference genome (mm10), the human C9orf72 sequence and the pCC1 vector used in transfection. Despite the fact that most duplicate sequences were removed in the initial processing of the data, additional duplicates were identified and marked by the MarkDuplicates tool from Picard (from the Broad Institute). Integration sites were identified by manual inspection of discordant read-pairs in which one end mapped to the mouse reference and the other end to the C9orf72 or pCC 1 sequences. Analysis of soft-clipped reads at the integration sites allowed determining, with base-pair resolution, the exact breakpoint location, in both the mouse genome and the C9orf72 sequence. Further analysis on the structure of the integration was based on read-pairing information and on copy-number analysis of the C9orf72 and pCC 1 sequences. For the latter, both the C9orf72 and the pCC 1 sequences were segmented based on integration breakpoints and deletion site boundaries, and, for each segment, average depth of coverage was computed using the Genome Analysis Toolkit (GATK). The obtained values were then compared to the average depth of coverage over the mouse reference, estimated from a set of intervals overlapping genic regions. For calling variants over both the C9orf72 and pCC 1 sequences, alignments from all four samples were combined into a single BAM file and GATK’s HaplotypeCaller was used to produce a gVCF file. Variants were then emitted by GATK’s GenotypeGVCFs.

### Transcriptome Sequencing and Data Analysis

Animals were perfused transcardially with sterile 1× PBS and the frontal cortex was harvested for RNA isolation. RNA was isolated using the Direct-Zol RNA miniprep kit (Zymo Research). RNA-seq libraries were prepared from total RNA (500-700 ng) using the directional RNA-seq kit with NEB Ultra Ribominus (New England Labs) per the manufacturer’s protocol. The RNA was then sequenced using the Illumina NextSeq500 machine at the Center for NeuroGenetics, University of Florida.

### RNA Sequencing Data Analysis

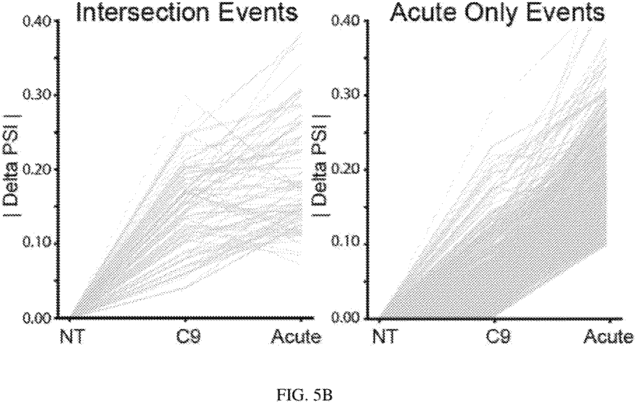

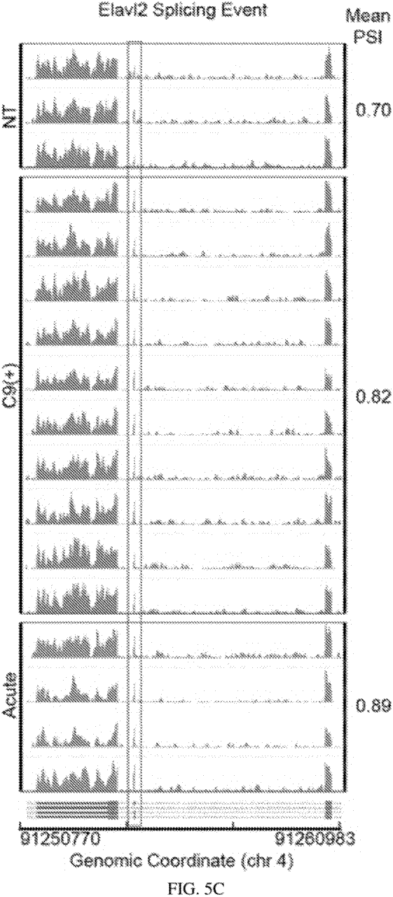

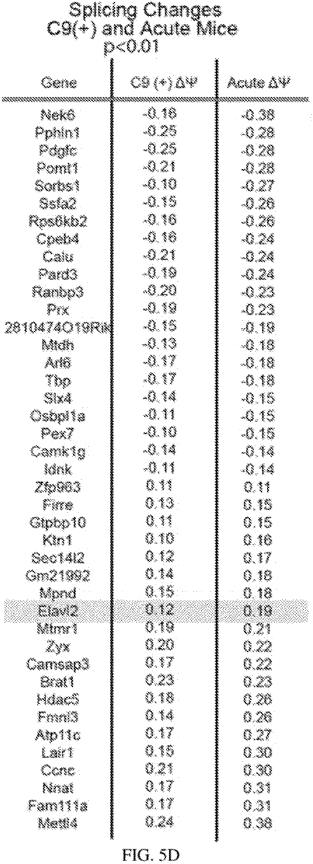

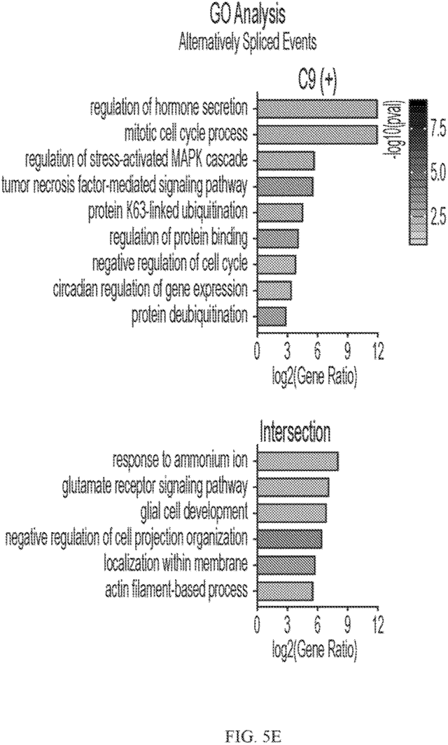

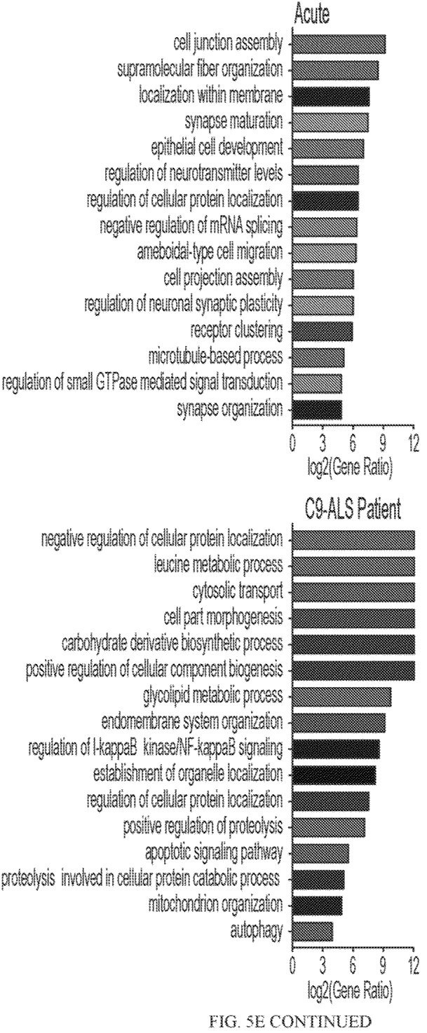

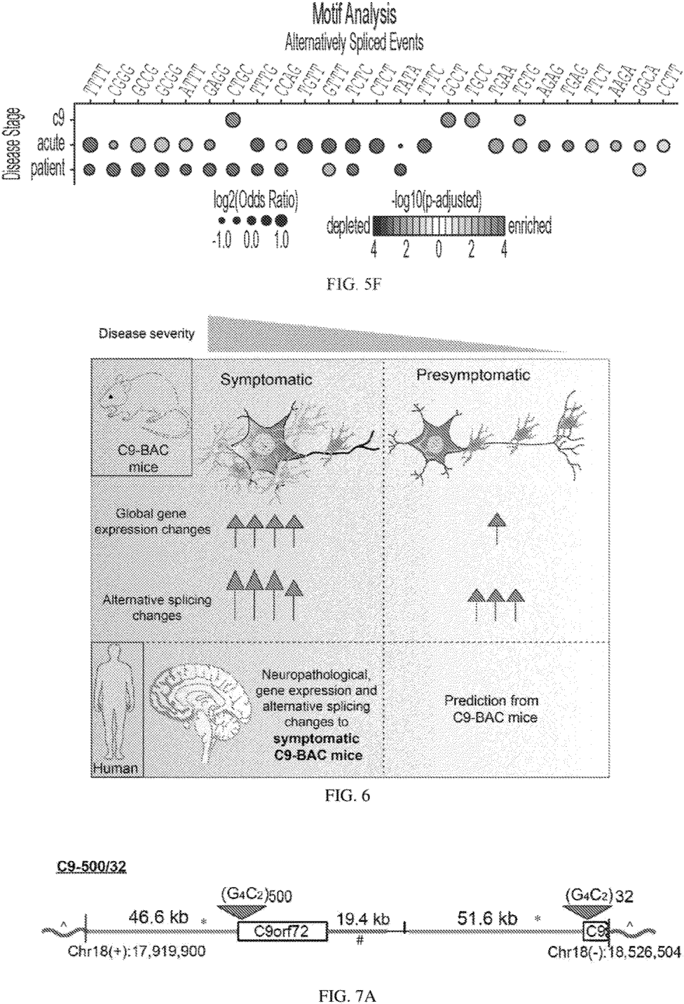

Reads obtained after sequencing were aligned using Spliced Transcripts Alignment to a Reference (STAR) to either the human genome (hg19) or mouse genome (mm10). Quality analysis of the reads was performed using RSeQC. Kallisto was used to obtain transcript per million (TPM) values for some analyses (example FIGS. 5A-5F and FIG. 6). GENCODE was used for gene annotations from STAR alignment wand RefSeq was used for Kallisto gene annotations. Deseq2 was used to measure gene expression differences, run using the Bioconductor package for R. MISO was used, based on MISO annotations version 2.0, to identify changes in alternative splicing. Only events with Bayes factor ≥ 5 for at least one pair-wise comparison were considered. Events were filtered with ΔΨ| ≥ 0.10 compared to the NT mice and the Venn diagrams were plotted using Biovenn. To identify enriched biological pathways, gene ontology analysis was performed using Metascape using gene sets from GO biological processes and was confirmed using DAVID annotation using default statistical thresholds (P-value < 0.01, maximum enrichment > 1.5). Graphs were plotted on R using ggplot2. C9-ALS patient data was obtained from Prudencio, et al. Data from frontal cortex on autopsy tissue samples were filtered based on RIN scores and samples used were: C9(+)-ALS -SRR1927020, SRR1927022, SRR1927024, SRR1927026, SRR1927028, SRR1927030, SRR1927032 and SRR1927034; Control - SRR1927056, SRR1927058, SRR1927060, SRR1927062, SRR1927064, SRR1927066, SRR1927068, SRR1927070, SRR1927071.

### Cell Type Enrichment Analysis

The index for relative expression of genes in different cell types was set up using publicly available data on cell type enrichment in the mouse cortex. Since the differences between the different types of oligodendrocyte cell types were subtle, myelinating oligodendrocytes, oligodendrocyte precursor cells, and mature oligodendrocytes were combined to one cell type-“oligodendrocyte”. For a gene to belong to a particular cell type, expression had to be > 20FPKM and logFC between any sample and mean of all other samples > 2. A Bayesian statistical model was used to calculate the proportions of cell types within the current dataset. The python package Pymc3 was used to perform these analyses and Matplotlib was used to generate the graphs.

### Motif Analysis

To analyze the number of enriched motifs, the monotonically changing cassette exons were used from alternatively spliced events generated from MISO with a Bayes factor > 5 and |ΔΨ| ≥ 0.10 in NT and acute animals. Fishers exact test and Bonferroni correction were performed, and the p adjusted < 0.05 was set as the significance threshold. The regions 250 bp upstream, exon, and 250 bp downstream were used to count for all possible combination of 4-mers. Fisher exact test was used to identify motifs that are enriched on depleted specifically in acute mice when compared to non-transgenic (NT) mice.

### Splicing Analysis

For splicing analyses, mm 10 annotated events were run on MISO to identify alternatively spliced events and to identify skipped exon (SE) events. Delta PSI values for non-transgenic (NT) vs. C9 mean PSIs and NT vs. Acute mean PSIs were calculated and are reported in Tables 3 and 4. A Wilcoxon rank-sum test was also run for both comparisons. Delta PSI cutoff was set at 0.1 (or -0.1) and a rank sum p value < 0.05 was used to establish significance. GO analysis was run with metascape.org.

### Southern Blot

Tail genomic DNA extraction and Southern blot experiments were performed following previously published protocols. Briefly, 10 µg of genomic DNA extracted from the tail or brain was digested with EcoR1 and BamH1 overnight at 37° C. The digested gDNA samples were then run on a 0.7% agarose gel for ~5-6 hours, depurinated (0.2 N HCl), denatured (1.5 M NaCl, 0.5 M NaOH), and neutralized (1.5 M NaCl, 0.5 M Tris HCl) for 15 minutes each. DNA was transferred overnight by capillary blotting to a positively charged nylon membrane and was cross-linked the next morning. For hybridization, the membrane was prehybridized for 1 hour using Amersham Rapid-Hyb buffer (GE Healthcare). The probe was labeled using dCTP-P³² using the random primed DNA labeling kit (Invitrogen) and was hybridized with the membrane. After 3 hours of hybridization, the membrane was washed with 2× SSC, 0.1% SDS for 20 minutes at room temperature, and then with 0.2x SSX, 0.1% SDS solution at 65° C. two times for 15 minutes each. Radioactivity was visualized on an X-ray film that is sensitive for P³² after 2-3 days of exposure at -80° C.

### Quantitative Real Time PCR

Total RNA was isolated from mouse frontal cortex and spinal cord tissues with TRIzol (Invitrogen). Following DNase treatment (Ambion) using manufacturer’s protocol, cDNA was prepared using the SuperScript III RT kit (Invitrogen) and random-hexamer primers (Applied Biosystems). Two-step quantitative RT-PCR was performed on a MyCycler Thermal Cycler system (Bio-Rad) using SYBR Green PCR Master Mix (Bio-Rad). See primer lists in Table 1.

TABLE 1

Primers used for PCR

| Primer name | Sequence | SEQ ID NO: |
|---|---|---|
| Mppel-F | GGCACCATGTCTGGTTTGTT | 14 |
| Mppe1-R | AGACTGGTCGGTGATCTCGT | 15 |
| Probe-F | AGAACAGGACAAGTTGCC | 16 |
| Probe-R | AACACACACCTCCTAAACC | 17 |
| C9GT-F | AGTTGGGTCCATGCTCAACAA | 18 |
| C9GT-R | ACTGTTCTAGGTACCGGGCT | 19 |
| 1a-F | GCCCACGTAAAAGATGACGC | 20 |
| 1a-R | CCTCCTAAACCCACACCTGC | 21 |
| AS-F | AGTCGCTAGAGGCGAAAGC | 22 |
| AS-R | CGAGTGGGTGAGTGAGGAG | 23 |
| C9mRNA-F | TCTCCAGCTGTTGCCAAGAC | 24 |
| C9mRNA-R | TCCATTCTCTCTGTGCCTTCT | 25 |
| mACT-F | TCGTGCGTGACATCAAAGAG | 26 |
| mACT-R | GATCTTCATGGTGCTAGGAG | 27 |

### Mouse Generation and Maintenance

C9-BAC mice transgenic mice were crossed with FVB mice obtained from Jackson Laboratory. Pups were genotyped based on genomic DNA extracted from the tail using the primers C9-GT F and R (Table 1), and the previously described protocol. Behavioral tests were performed at 12, 16, and 24 weeks of age. Digigait and open field analyses were performed based on the manufacturer’s protocol. Mice were perfused transcardially using 1× PBS and were embedded in 10% formalin or OCT frozen in cold 2-methylbutane for further analyses.

### Fluorescence In Situ Hybridization (FISH)

10 µm frozen sections embedded in OCT were cut on the cryostat. Frozen sections were fixed in 4% PFA in PBS for 20 minutes and incubated in pre-chilled 70% ethanol for 30 minutes or longer at 4° C. Following rehydration in 40% formamide in 2× SSC for 10 minutes, the slides were pre-hybridized with hybridization solution (40% formamide, 2× SSC, 20 µg/mL BSA, 100 mg/mL dextran sulfate, and 250 µg/mL yeast tRNA, 2 mM Vanadyl Sulfate Ribonucleosides) for 30 minutes at 55° C. and then incubated with 200 ng/mL of denatured DNA probe ((C2G4)₃-Cy3 for sense foci and G4C2₃-Cy3 for antisense foci) in hybridization solution at 55° C. After 3 hours of hybridization, the slides were washed three times with 40% formamide in 2× SSC and briefly washed one time in PBS. Autofluorescence of lipofuscin was quenched by 0.25% of Sudan Black B in 70% ethanol. Slides were mounted with mounting medium containing DAPI (Invitrogen) and imaged on LSM880 Confocal microscope.

### Immunohistochemistry

Five-micrometer sections were deparaffinized in xylene and rehydrated through graded ethanol solution. Antigen retrieval was performed by incubating the slides in a steamer with 10 mM citrate buffer (pH 6.0) for 30 minutes or with 10 mM EDTA (pH 6.4) for 12 minutes. The slides were cooled down to room temperature, and then washed for 10 minutes in running tap water. The slides were incubated in 95-100% formic acid for 5 minutes and subsequently washed for 10 minutes in running tap water. To eliminate nonspecific binding and excessive background, slides were blocked with a serum free block or rodent block (Biocare Medical) for 15 minutes. Primary antibody diluted in 1:10 blocking solution was applied on the slides and incubated overnight at 4° C. (see below for dilution information). Slides were washed three times with 1× PBS and incubated with linking reagent (streptavidin or alkaline phosphatase; Covance) or biotinylated rabbit anti-goat IgG (Vector Labs) for 30 minutes at room temperature. After washing with 1× PBS, these sections were then incubated in 3% H₂O₂ (in methanol) for 15 minutes to eliminate any endogenous peroxidase activity.

After washing in running tap water for 15 minutes, labeling reagent (HRP, Covance; Vectastain ABC-AP kit) was then applied to the slides for 30 minutes at room temperature. The slides were developed with NovaRed and DAB (Vector Labs) to measure the peroxidase activity and the slides were then counterstained with hematoxylin (modified Harris, Sigma Aldrich), rehydrated in graded alcohol and cover-slipped for visualization. Images were taken on the Olympus BX51 microscope using the Cellsense software. For hematoxylin and eosin staining, the slides were deparaffinized in xylene and dehydrated through graded ethanol solution. The slides were then soaked in hematoxylin (modified Harris, Sigma Aldrich) for 1 minute and washed in running distilled water for 10 minutes. Next, the slides were immersed in Eosin Y (Sigma Aldrich) for 30 seconds and washed in distilled water for 10 minutes. The slides were rehydrated and cover-slipped before visualization. For cresyl violet staining, slides were deparaffinized in xylene and subsequently rehydrated in graded ethanol solution. The slides were incubated in 0.25% cresyl violet at 60° C. for 8-10 minutes and differentiated in 95% ethanol for 1-5 minutes. Slides were then immersed in 100% ethanol and xylene and cover-slipped for visualization.

### Immunofluorescence

Frozen sections were warmed up at room temperature for 2 hours. Slides were fixed with 4% PFA at room temperature for 10 minutes and immediately permeabilized with ice-cold 1:1 methanol-acetone for 10 minutes at -20° C. Slides were blocked with background sniper at room temperature for 30 minutes to 1 hour and incubated overnight with primary antibody (polyGA 1:2000, poly GP 1:1000, generously donated by Neurimmune, Inc.) prepared in a 1:10 dilution of background sniper. The slides were washed with 1× PBS the next day and incubated with secondary antibody (Cy3-anti-human IgG antibody, 1:5000) for 1-2 hours. The slides were washed with 1× PBS and mounted with Prolong with DAPI. Slides were imaged with LSM880.

### Western Blot

Brain lysates were solubilized with RIPA buffer (1% sodium deoxycholate, 1% Triton X-100, 50 mM Tris pH 7.5, 150 mM NaCl and proteinase inhibitor). 10 µl of lysates were run on a 4-12% Bis-Tris gel and transferred to a nitrocellulose membrane. The membrane was blocked in 5% milk diluted in PBST (1× PBS with 0.05% Tween-20). The membrane was then incubated with the primary antibody (α-C9orf72, 1:1000) overnight at 4° C. The blot was washed three times with 1× PBS the next morning and was incubated with secondary antibody for 1 hour at room temperature. The membrane was developed using ECL prime and was visualized for signal.

### Image J Quantification

Quantification was performed by two independent blinded investigators using the cell counter plugin in Image J (National Institute of Health). Serial sections 20-30 µm apart were stained, imaged, and quantified for subsequent analyses.

### Behavioral Analyses

DigiGait and Openfield Analyses were performed as previously described in Liu, et al., 2016. Scoring criteria for cage behavioral assessments were as in Liu, et al.

### Statistics

GraphPad Prism 7 was used to perform the statistical analyses in the manuscript. Significance threshold was set at p < 0.05. The significance values were set as ns = not significant, p > 0.05, p < 0.05 - “*”, p < 0.01 - “**”, p < 0.001 - “***”, p < 0.0001 - “****”. Comparisons between groups was performed using mean + SEM values using one-way ANOVA and multiple comparisons. Significance in survival was measured using the Log Mantel-Cox test. Digigait analyses were performed using multiple comparisons

### Example 2: Phenotypes in C9-BAC Mice Independent of Integration Sites

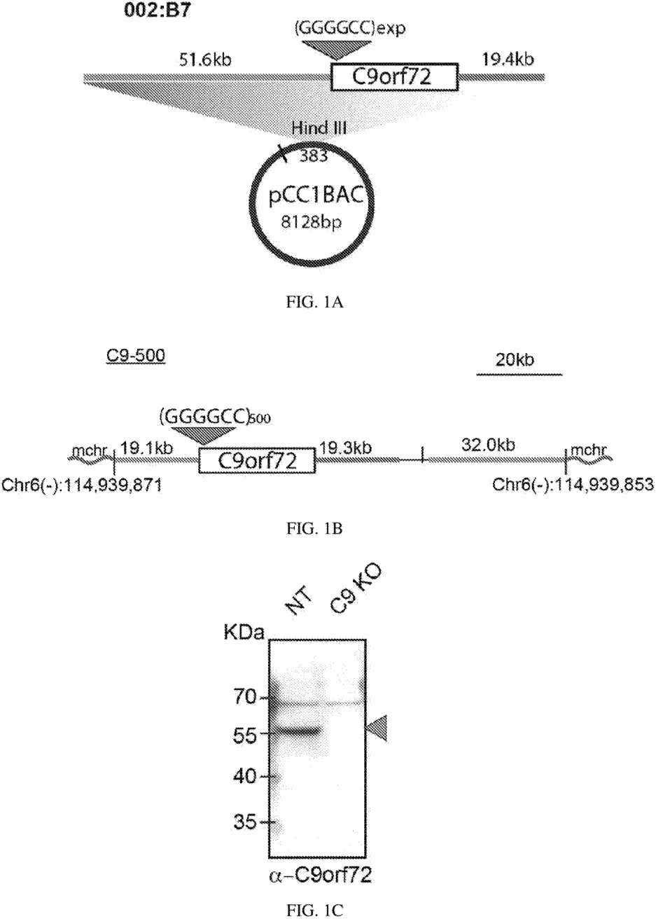

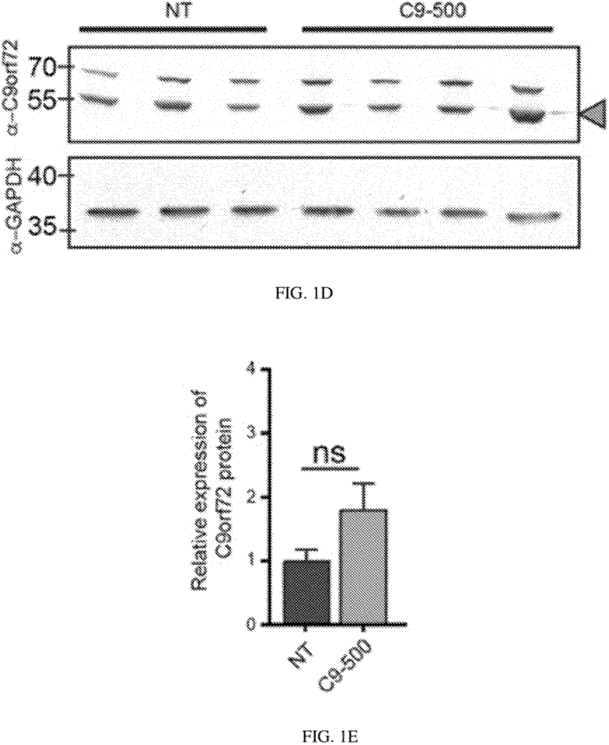

A BAC transgenic model of C9orf72 ALS/FTD was previously developed. Four independent lines were generated by pronuclear injection of a circularized BAC containing a 98.3 KB human DNA insert, including a large G₄C₂ repeat-expansion mutation as shown in FIG. 1A. To further characterize these mice, whole genome sequencing was performed to determine the transgene break points, genomic integration sites, and the number of transgene copies for each of the four BAC mouse lines. Transgene break and genomic integration sites were identified by computational analyses of discordant read-pairs from transgenic DNA compared to mouse and human reference genomes. Transgene copy number for each of the lines was determined by comparing the regional coverage depth of the BAC with the average coverage depth of the mouse reference genome. These data show that the transgenes in all four BAC lines were inserted into distinct single integration sites. Transgene copy number for each of the lines was consistent with the previous estimates based on Southern blot and qRT-PCR analyses.

In the single-copy C9-500 line, the transgene is inserted between Chr 6(-): 114,939,871 and Chr 6(-): 114,939,853 in the mouse genome resulting in an ~ 18 bp deletion in an intergenic region ~18.1 kb distal and ~64.5 kb proximal of the nearest flanking genes Vgll4 and Tamm41, respectively, as shown in FIG. 1B. The breakpoint on the circular BAC transgene occurred 19,097 bp upstream of C9orf72. At the site of integration, the C9-500 line contains the full length C9orf72 gene with -500 G₄C₂•G₂C₄ repeats with 19.1 kb of 5′ human flanking sequence and 19.4 kb of 3′ human flanking sequence. This is followed by additional sequence which includes the pCC 1BAC backbone and the 32.0 kb of human DNA originally 5′ of the breakpoint on the BAC. In summary, the C9-500 line has a single integration site containing a full-length copy of the C9orf72 gene, substantial human flanking sequence and ~500 G₄C₂•G₂C₄ repeats.

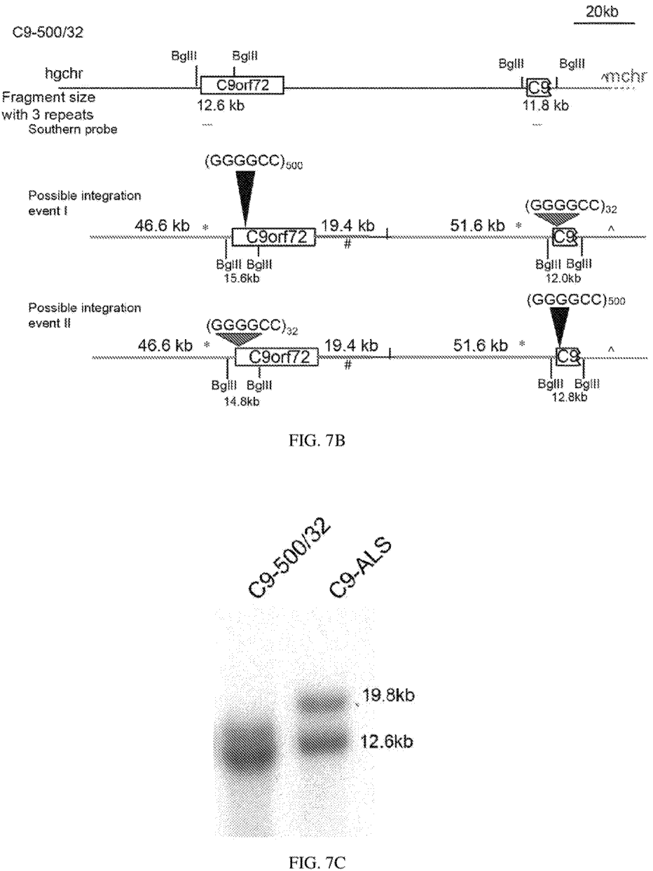

In the C9-500/32 line, two copies of the transgene were integrated between mouse chromosome 18 (-) 17,919,900, and the flipped chromosome 18 (+) strand at position 18,526,504, as shown in FIG. 7A. No annotated genes were found in this region and no decrease in coverage between the breakpoints was observed, indicating that a small mouse chromosomal rearrangement, but not a mouse chromosomal deletion, occurred in this region, as shown in FIG. 7A. In the C9-500/32 line, the first copy of the integrated human transgene contains 46.6 kb of upstream and 19.4 kb of downstream flanking sequences and the full-length copy of the C9orf72 gene. The second transgene copy contains the full 51.6 kb upstream flanking region and a small portion of C9orf72 which terminates 3′ of the repeat within the first intron. Southern blot analyses show that the larger repeat expansion is located in the full length C9orf72 copy and the shorter expansion in the second copy containing the truncated C9orf72 gene, as shown in FIGS. 7B and 7C.

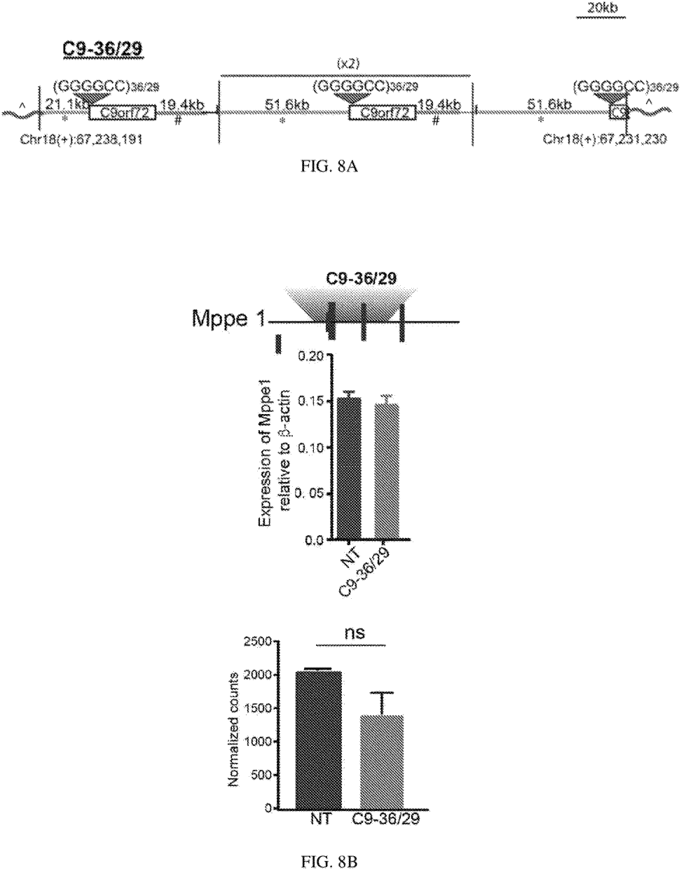

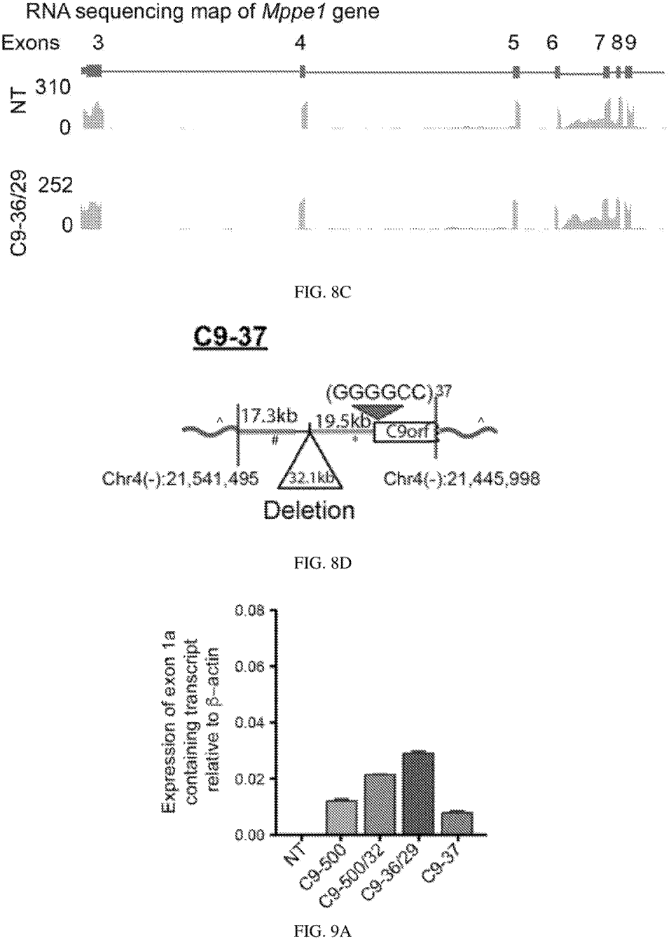

The C9-36/29 line has three copies of the full-length C9orf72 gene, and one truncated copy inserted into the first second intron of metallophosphoesterase 1 (Mppe1) (FIG. 8A). Although the deletion of exons 3-4 of Mppe1 is predicted to lead to the expression of a truncated protein lacking the N-terminal region and a portion of the metallophosphoesterase domain, qRT-PCR and RNA sequencing detected no significant differences in transcript levels including over exons 3 and 4 (FIGS. 8B, 8C). While it is possible that a truncated metallophosphoesterase protein is expressed and could cause some type of deleterious effect, no overt phenotypic differences were found in this line compared with the other phenotypic lines.

The C9-37 line has a single transgene insertion site on chromosome 4 at a position with no annotated genes. This insertion contains a partial copy of C9orf72 extending from exon 1 into intron 9 plus 19.5 kb of endogenous human upstream flanking sequence. Due to the position of the transgene break, the pCC1BAC backbone plus an additional 17.3 kb of 3′ sequence is integrated further upstream of C9orf72 (FIG. 8D). The C9-37 line, which lacks the 3′ end of C9orf72, is the only line that does not contain a full-length copy of the transgene, and the only line that does not develop overt ALS/FTD phenotypes.

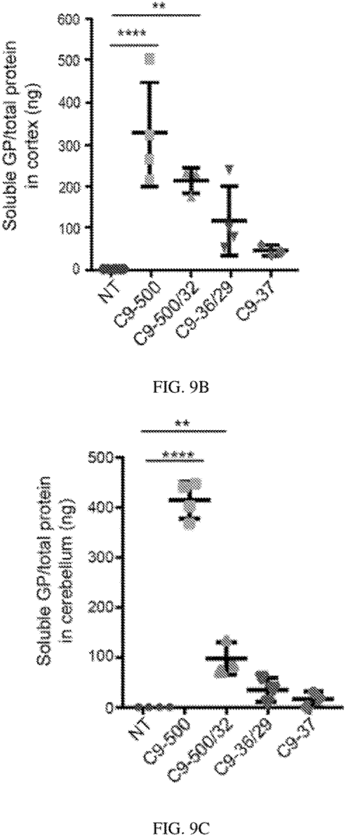

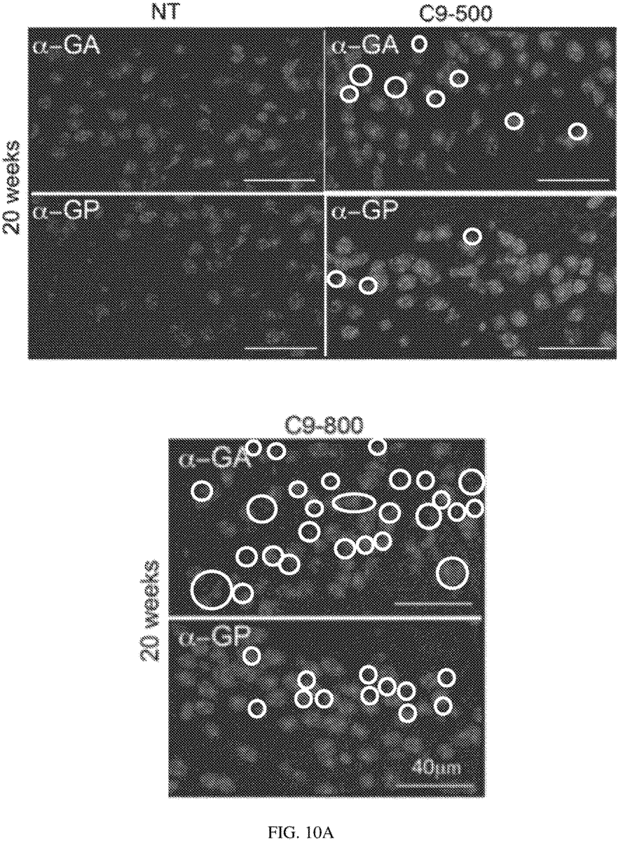

The levels of exonla containing sense RNA transcripts were measured by RT-qPCR and polyGP RAN protein using a meso scale discovery (MSD) assay (FIGS. 9A-9C). RT-qPCR shows that the levels of sense expansion containing RNA transcripts in the brain from each of four C9-BAC lines correlate with transgene copies (FIG. 9A). In contrast, the levels of polyGP RAN proteins are the highest in the most penetrant C9-500 line in both the cortex and cerebellum (FIG. 9B). FIG. 9C shows the use of an MSD immunoassay to measure levels of soluble GP in cerebellar brain lysates.

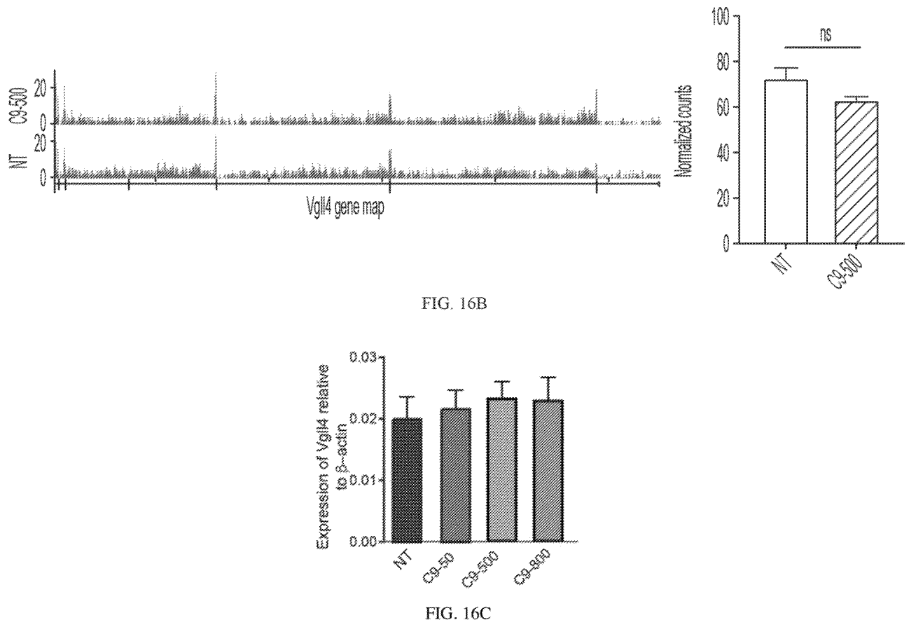

FIGS. 16A-16C show Vgll4 expression in non-transgenic (NT) animals, or animals with C9-50, 500 or 800 repeats. FIG. 16A is a schematic showing the location of the transgene in Vgll4 gene, and qRT-PCR comparing expression levels of Vgll4 in NT and C9-500 mice. FIG. 16B depicts a histogram showing coverage of reads over the first exon and intron of Vgll4 gene. The bar graph shows the normalized counts from the RNA sequencing data set (n=3 animals per group). qRT-PCR and RNA sequencing showed no apparent differences in the levels of Vgll4 in C9-500 compared to NT mice. FIG. 16C depicts qRT-PCR, which shows expression of Vg114 relative to β-actin. qRT-PCR shows that Vgll4 expression was not different in animals with C9-50, 500 or 800 repeats.

In summary, three independent C9orf72 BAC lines show similar ALS/FTD phenotypes, strongly supporting the hypothesis that these phenotypes are caused by the repeat expansion mutation and not disruptions of integration site genes or other changes at the various integration sites. Of the two lines with relatively short repeats (C9-36/29 vs C9-37), only the C9-36/29 line containing 4 copies of transgenes develops ALS/FTD phenotypes. This difference could result from the higher expression of the relatively small G₄C₂ expansions in the phenotypic C9-36/29 line (4 transgene copies) compared with the C9-37 line (1 transgene copy) or the truncation of the C9orf72 gene found in the C9-37 line.

Multiple lines of C9orf72 BAC transgenic mice were previously reported to develop the molecular, behavioral, and neuropathological features of ALS/FTD. Transgene insertion sites are now described, and it is shown that these insertions do not disrupt endogenous gene expression. To understand the effects of repeat length on disease, an isogenic allelic series of mice with 800, 500 or 50 repeats was developed, and it was demonstrated that longer repeats result in earlier onset and increased penetrance. Transcriptomic profiles of C9-500 mice at end-stage are similar to human C9-ALS autopsy tissue, while alternative splicing changes occur prior to disease onset and worsen with disease progression. Finally, C9orf72 protein levels are lower while DPR levels are higher in the C9-500 mice compared to an independent asymptomatic C9-BAC model. These data demonstrate repeat length is an important driver of disease and that the C9-BAC mice are a useful tool for understanding disease mechanisms and identifying biomarkers of C9orf72 ALS/FTD.

### Example 3: Single-Copy C9-500 Mice Express Higher GP Levels Than Mixed Repeat Length, High-Copy Baloh-Jax Mice

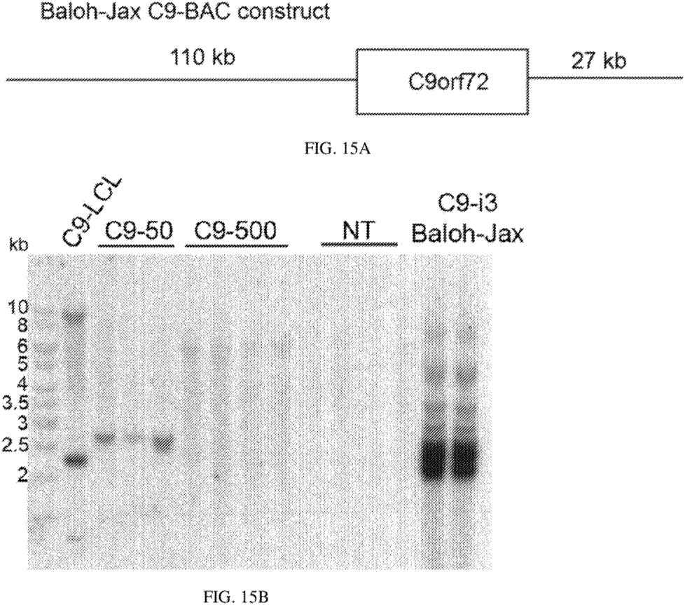

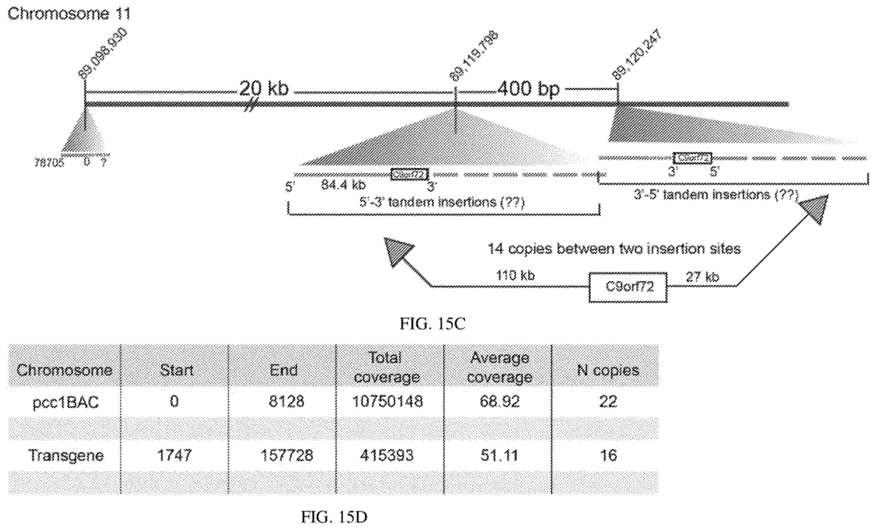

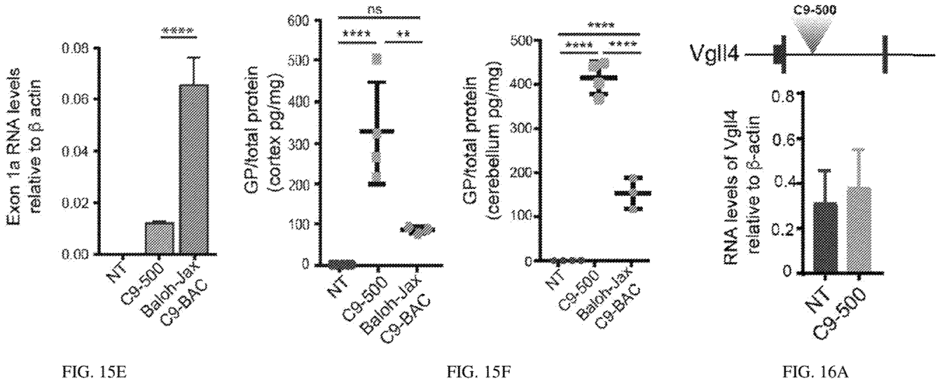

Similar to the mice described above, an independent C9-BAC mouse model using a BAC construct with the full-length C9orf72 gene plus substantial flanking sequence was developed (FIG. 15A). In contrast to the BAC transgenic model of C9orf72 ALS/FTD described above, the independent C9-BAC mouse model lines do not develop behavioral or neurodegenerative features of C9orf72 ALS/FTD. To understand the molecular reasons for these phenotypic differences, a series of experiments was performed comparing the single-copy C9-500 line and the Baloh-Jax C9-BAC mice using a C9-i3 expansion line from the independent C9-BAC mouse model. Southern analyses show that the Baloh-Jax C9-BAC mice have a much higher signal than the single copy C9-500 line but most of this signal is found between 2 and 2.5 kb, a size range associated with alleles with relatively short repeats (~3-50). Additionally, several lighter bands are seen at higher molecular weights, ranging in size from 3-7 kb (FIG. 15B).

Whole genome sequencing (WGS) of DNA from the Baloh-Jax mice shows three different insertion sites on chromosome 11, with two sites likely containing a total of 14 additional tandem insertions in two closely spaced sites in the 5′ to 3′ or 3′ to 5′ direction of the transgene (FIG. 15C). Copy number estimates based on sequencing analyses predict a total of 16 copies of the transgene inserted at two closely spaced integration sites on chromosome 11 (Site A - position: 89,119,798 or Site B - position 89,120,247) shown in FIG. 15D. Consistent with the high transgene copy number, qRT-PCR shows that the expression of Exon la repeat containing sense transcripts are higher in the Baloh-Jax mice compared to the C9-500 line (FIG. 15E). In contrast, MSD immunoassays show soluble GP levels are lower in the Baloh-Jax mice compared to the C9-500 mice in both the cortex and cerebellum (FIG. 15F). These data and the Southern blot data suggest that most of the C9 transcripts expressed in Baloh-Jax mice have shorter repeats, and are less likely to produce RAN proteins. The higher levels of RAN protein observed in the C9-500 compared to the Baloh-Jax mice may contribute to the ALS/FTD behavioral and neurodegenerative phenotypes that are seen in the C9-500 mice, but not the Baloh-Jax mice.

### Example 4: C9orf72 Protein Overexpression is Not Associated With Disease in C9-500 Mice

To study if the integration of the transgene results in an upregulation of C9orf72 protein levels that might contribute to phenotypes seen in the C9-500 line, the levels of the C9orf72 protein in 20 week old C9-500 mice were compared with age-matched non-transgenic (NT) mice. Splice variants of the C9orf72 mRNA generate two isoforms of the protein with predicted sizes of ~55 kDa and -35 kDa. Commercially available antibodies detect the long isoform. The C9orf72 Genetex antibody that detects both human and mouse C9orf72 proteins was used, and antibody specificity was confirmed by showing that the 55 kDa protein is detected in the control, but not C9orf72 KO brain lysates (FIG. 1C). While the levels of the C9orf72 protein trended towards an increase in C9-500 mice, no significant upregulation was detected in cortical brain lysates from C9-500 compared to NT mice (FIGS. 1D-1E).

In summary, the phenotypic C9-500 mice show modest but insignificant elevation of C9orf72 protein, suggesting that this change is unlikely to be a critical driver of the ALS/FTD phenotypes in this mouse model.

### Example 5: Repeat Length Increases Penetrance and Decreases Survival in Allelic Series of C9-BAC Mice

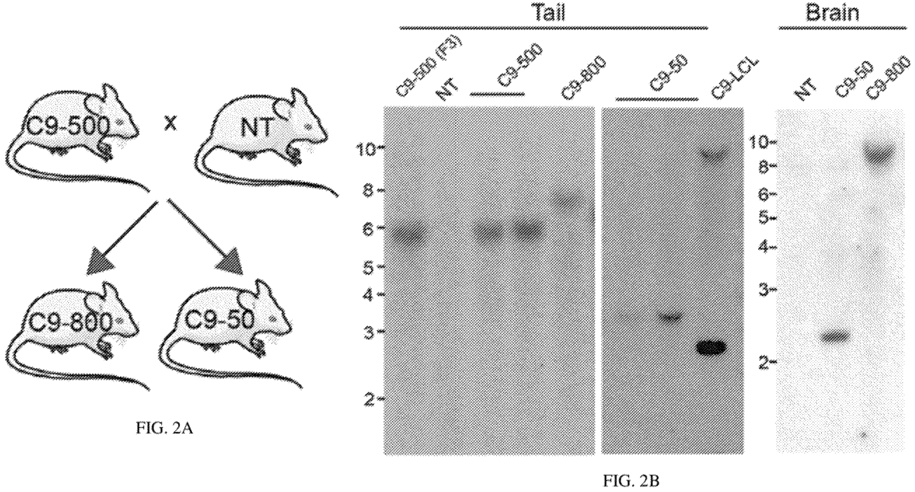

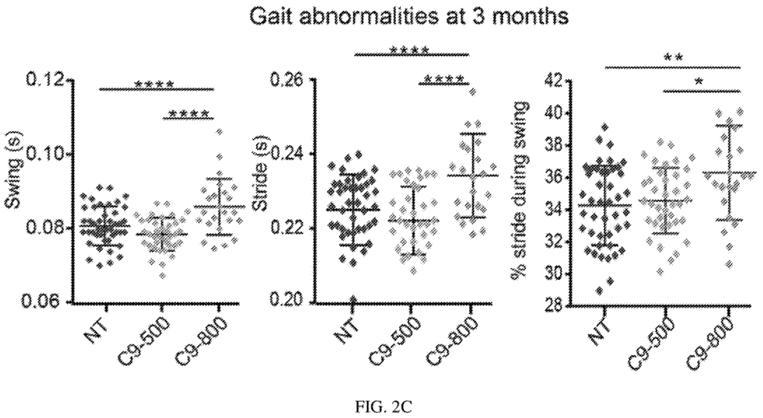

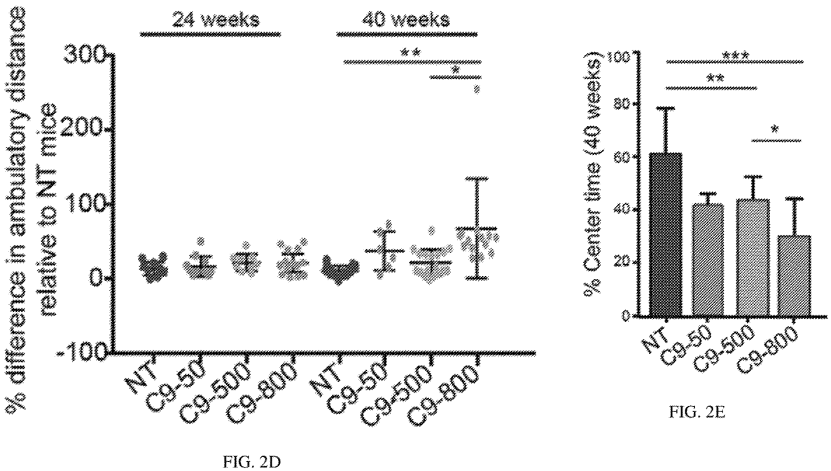

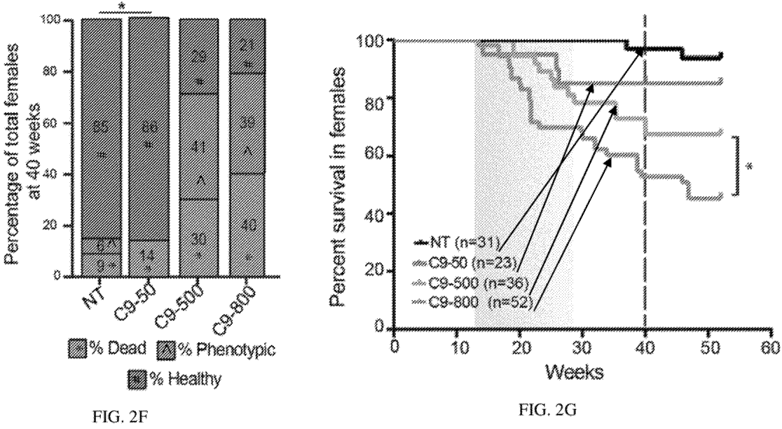

Repeat length is a known modifier of disease severity in multiple repeat expansion diseases including Huntington disease, DM1 and multiple spinocerebellar ataxias. Somatic instability and technical difficulties in measuring G₄C₂ repeat length in human C9orf72 patients have made the contribution of repeat length to age of onset and disease severity of C9orf72 ALS/FTD difficult to determine. To test the hypothesis that the length of the G₄C₂ repeat is an important modifier of age of onset and disease risk, an allelic series of mice was established from the most penetrant BAC transgenic line (C9-500). Taking advantage of the intergenerational repeat instability observed during the maintenance of the colony, C9-500 animals that had repeat contractions or expansions were selected and bred, and sublines were established with 50 (C9-50) or 800 (C9-800) repeats (FIGS. 2A, 2B). Limited somatic repeat instability was observed between tail and brain DNA from the 50 and 800 lines (FIG. 2B). The limited somatic instability of the G₄C₂•G₂C₄ expansion, the single-copy of the C9orf72 transgene, and the identical insertion site shared by the C9-800, C9-500, and C9-50 sublines make these mice an ideal resource to test the effects of repeat length on age of onset and disease penetrance.

To understand the effects of repeat length on disease, a series of experiments were performed on female mice from this allelic series. First, DigiGait analyses were performed at an early time-point to test if phenotypes in the C9-800 mice were detected earlier than in the C9-500 cohort. At 12 weeks of age, C9-800 mice showed abnormalities in 9 of 42 DigiGait parameters, while the C9-500 mice showed 6 differences compared to NT mice (Table 2). Additionally, 12 parameters were different between C9-800 and C9-500 mice, (FIG. 2C) including three key parameters typically involved in ALS: swing time, stride time, and time of a stride when paw is in swing motion. These data show gait abnormalities are more prevalent in mice with longer repeats.

Open field studies previously showed both slowed movement and hyperactivity in old C9-500 mice (12-18 months). To test if increased repeat length causes earlier open field abnormalities, the allelic series of mice were examined at 24 and 40 weeks of age. At 24 weeks there were no differences in ambulatory distance between any of the C9-50, C9-500 and C9-800 sublines compared to NT animals. In contrast, at 40 weeks the C9-800 group showed a significant increase in ambulatory distance compared to both the C9-500 and NT cohorts (FIG. 2D). In contrast, no significant differences were found between the C9-500 or C9-50 animals and NT controls at this age. Open-field analyses were also used to measure decreases in center time, a phenotype associated with anxiety-like behavior. At 40 weeks, both the C9-500 and C9-800 animals showed decreased center time compared to NT controls. Additionally, center time in the C9-800 animals was decreased compared to C9-500 mice (FIG. 2E). No differences in center time were observed in C9-50 compared to NT controls. Taken together, these data show open field abnormalities are exacerbated with increased repeat length.

Phenotypic mice show abnormal cage behavior including kyphosis, inactivity, severe dehydration, and hind-limb paralysis. Population census and cage behavior analyses, which compare the percentages of dead, phenotypic, and apparently healthy animals at 40 weeks, show significant differences between the C9-500 and C9-800 cohorts as compared to non-transgenic (NT) controls. In contrast, C9-50 animals were not significantly different from NT animals (FIG. 2F). Although the overall phenotype distributions in the C9-50 cohort were not significantly different from the NT mice, several mice in the C9-50 line died with features of ALS/FTD including paralysis, kyphosis, weight loss, and neurodegeneration. These data demonstrate that repeats as short as 50 can cause C9orf72 ALS/FTD phenotypes, but with reduced penetrance compared to the C9-500 and C9-800 lines. Death in the C9-500 line typically begins at ~20 weeks of age. In contrast, death of animals in the C9-800 line begins earlier, at approximately 12 weeks of age. Kaplan Meier analyses show a significant decrease in survival in the C9-800 line compared to C9-500 animals by 52 weeks (FIG. 2G). Taken together, these data demonstrate that increased repeat length leads to increased penetrance and earlier disease onset.

TABLE 2

DigiGait parameters in 12-week old females.

| | | | | |
|---|---|---|---|---|
| Parameters (41 in total) | Unit | NT vs C9-500 | NT vs C9-800 | C9-500 vs C9-800 |
| Significant Parameters (Desired FDR=5%) | (real#) | 6 | 9 | 12 |
| Swing | (s) | ns | ^{∗∗∗∗}p<0.0001 | ^{∗∗∗∗}p<0.0001 |
| Swing/Stride | (%) | ns | **p=0.0052 | *p=0.0176 |
| Brake | (s) | ns | ns | ns |
| Brake/Stride | (%) | ns | ns | ns |
| Propel | (s) | ns | ns | *p=0.0255 |
| Propel/Stride | (%) | ns | ns | ns |
| Stance | (s) | ^{∗}p=0.0444 | ns | ^{∗∗}p=0.0020 |
| Stance/Stride | (%) | ns | ^{∗∗}p=0.0052 | ^{∗}p=0.0174 |
| Stride | (s) | ns | ^{∗∗∗∗}p<0.0001 | ^{∗∗∗∗}p<0.0001 |
| Brake/Stance | (%) | ns | ns | ns |
| Propel/Stance | (%) | ns | ns | ns |
| Stance/Swing | (real#) | ns | ^{∗}p=0.0134 | ns |
| Stride Length | (cm) | ns | ^{∗∗∗∗}p<0.0001 | ^{∗∗∗∗}p<0.0001 |
| Stride Frequency | (steps/s) | ^{∗}p=0.0292 | ^{∗∗∗}p=0.0001 | ^{∗∗∗∗}p<0.0001 |
| Paw Angle | (deg) | ns | ns | ns |
| Absolute Paw Angle | (deg) | ns | ns | ns |
| Paw Angle Variability | (deg) | ns | ns | ns |
| Stance Width | (cm) | ^{∗}p=0.0292 | ns | ns |
| Step Angle | (deg) | ns | ns | ns |
| Stride Length Variability | (cm) | ns | ns | ns |
| Stance Width Variability | (cm) | ^{∗∗}p=0.059 | ns | ns |
| Step angle Variability | (deg) | ns | ns | ^{∗}p=0.0283 |
| #Steps Stride Length CV | (real#) (CV%) | ns ^{∗∗∗∗}p<0.0001 | ns ^{∗∗∗∗}p<0.0001 | ns ns |
| Stance Width CV | (CV%) | ^{∗∗∗}p=0.0006 | ns | ^{∗}p=0.0243 |
| Step Angle CV | (CV%) | ns | ns | ^{∗}p=0.0202 |
| Swing Duration CV | (CV%) | ns | ns | ns |
| Paw Area at Peak Stance | (cm2) | ns | ns | ns |
| Paw Area Variability at Peak Stance | (cm2) | ns | ns | ns |
| Hind Limb Shared Stance Time | (s) | ns | ns | ns |
| Shared/Stance | (%) | ns | ns | ns |
| Stance Factor | (real#) | ns | ns | ns |
| Gait Symmetry | (real#) | ns | ^{∗∗∗}p=0.0001 | ^{∗∗∗∗}p<0.0001 |
| MAX dA/dT | (cm²/s) | ns | ns | ns |
| MIN dA/dT | (cm²/s) | ns | ns | ns |
| Tau - Propulsion | (real#) | ns | ns | ns |
| Overlap Distance | (cm) | ns | ns | ns |
| Paw Placement Positioning | (cm) | ns | ns | ns |
| Ataxia Coefficient | (real#) | ns | ns | ns |
| Midline Distance | (cm) | ns | ns | ns |
| Paw Drag | (mm²) | ns | ns | ns |

#, number; CV, coefficient of variation; ns, not significant in one way ANOVA; desired FDR (false discovery rate) set to 5%; NT, n=44; C9-500, n=38; C9-800, n=24.

### Example 6: RNA Foci and RAN Protein Aggregates Increase With Increased Repeat Length in C9-BAC Mice

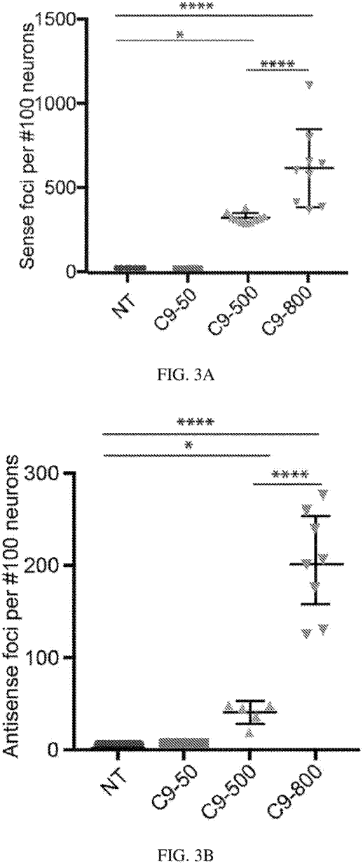

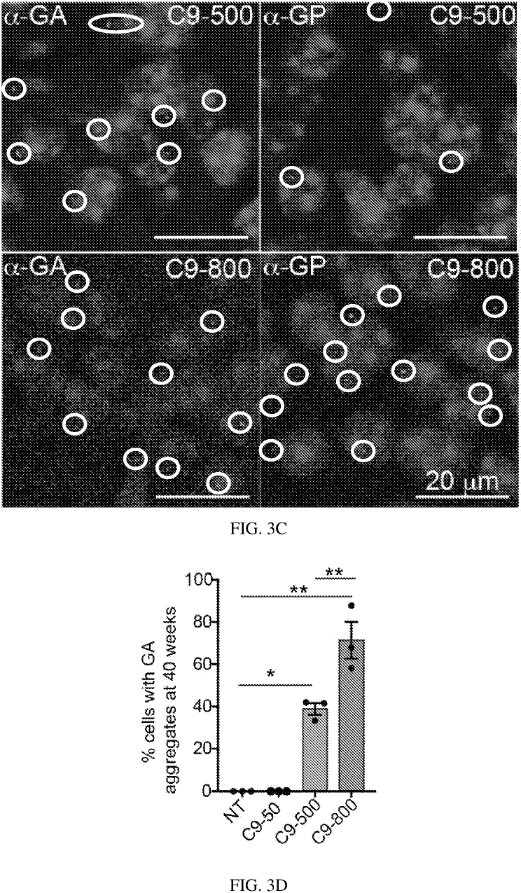

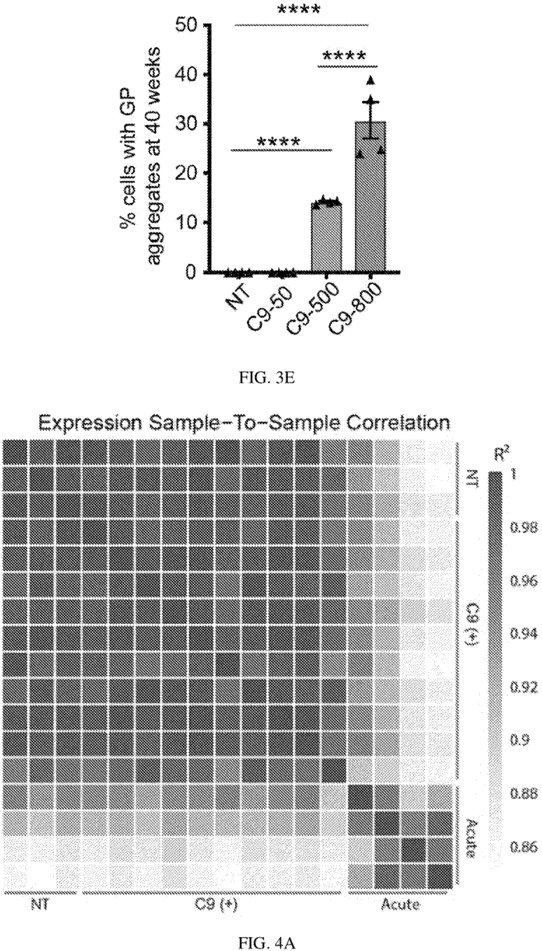

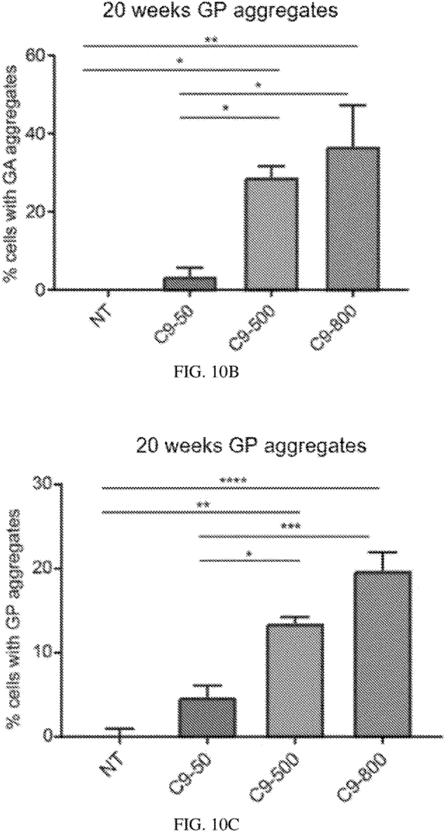

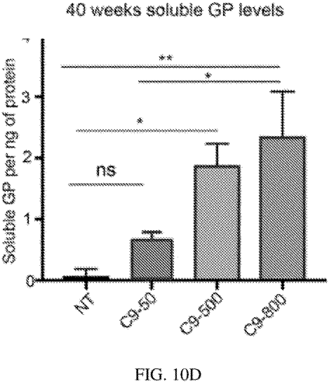

Next, a series of experiments was performed to understand the molecular changes associated with increases in repeat length. At 20 weeks of age, there was a significant increase in sense and antisense foci in the C9-800 dentate gyrus compared to the C9-500 line (FIGS. 3A, 3B). Similar to data on mice with shorter repeat tracts (29-37 repeats), sense and antisense RNA foci were not detected in animals in the C9-50 line (FIGS. 3A, 3B). Next, RAN protein aggregates in the retrosplenial cortex were compared by immunohistochemistry (IHC) or immunofluorescence (IF) using human a-GAi and a-GP₁ antibodies (FIGS. 3C, 10B). At 40 weeks of age, there was a significant increase in percentage of cells with polyGA aggregates (71% vs. 40%) and polyGP aggregates (22% vs. 15%) detected by IHC or IF in the C9-800 compared to the C9-500 mice (FIGS. 3C-3E). FIG. 10A shows representative images of GA and GP aggregates in the retrosplenial cortex of C9 BAC mice. No GA or GP aggregates were detected in the C9-50 sub-line, consistent with the decreased penetrance in this sub-line. IF studies performed at 20 weeks of age showed similar trends with significantly higher levels of aggregates between the C9-800 vs. non-transgenic (NT) and C9-500 vs. NT, but no difference between these groups (FIGS. 10B, 10C). The levels of soluble GP protein measured by MSD showed similar trends at 40 weeks of age (FIG. 10D).

In summary, these data demonstrate that repeat length is a modifier of disease in the C9-BAC transgenic mouse model. Mice with longer G₄C₂ repeat tracts show increased disease penetrance and earlier ages of onset and are characterized by increased levels of RNA foci and RAN protein aggregates.

### Example 7: Neuroinflammatory Transcriptome Changes Predominate at End-Stage

RNA dysregulation is thought to play a role in C9orf72 ALS/FTD, but little is known about how the transcriptome is affected early in the course of disease or how these changes progress over time. To look for transcriptomic abnormalities that occur during disease progression, RNA sequencing was performed on frontal cortex samples from ten female C9-500 mice at 20 weeks of age with no overt cage behavior abnormalities (C9+ pre-symptomatic), four C9+ animals that developed acute rapidly progressive phenotypes (20-22 weeks old) (acute), and three non-transgenic (NT) controls.

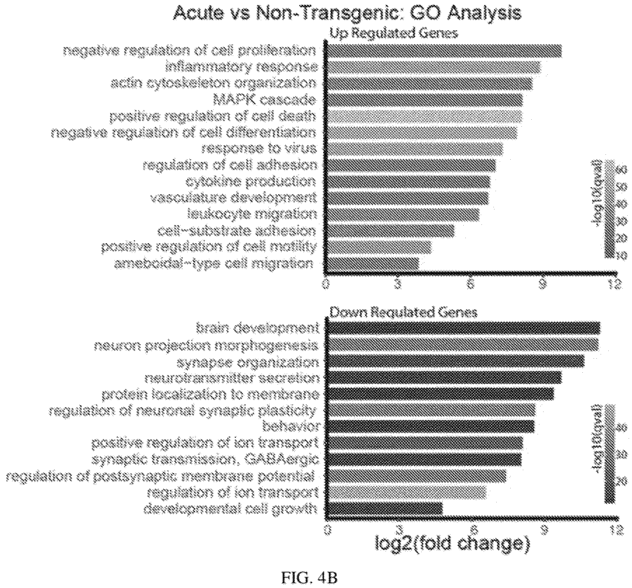

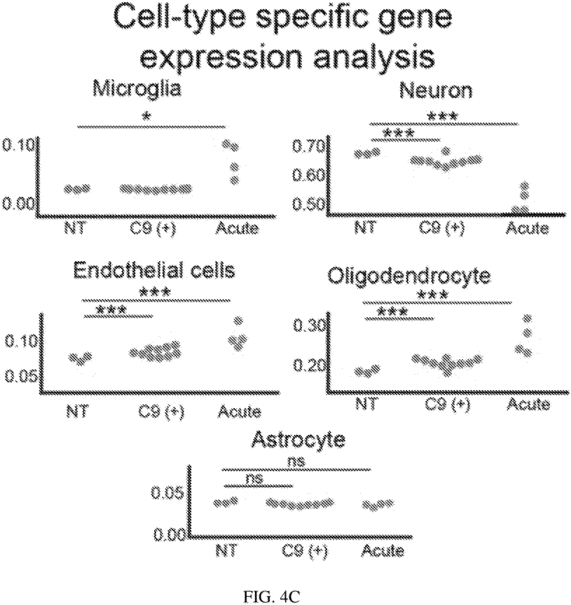

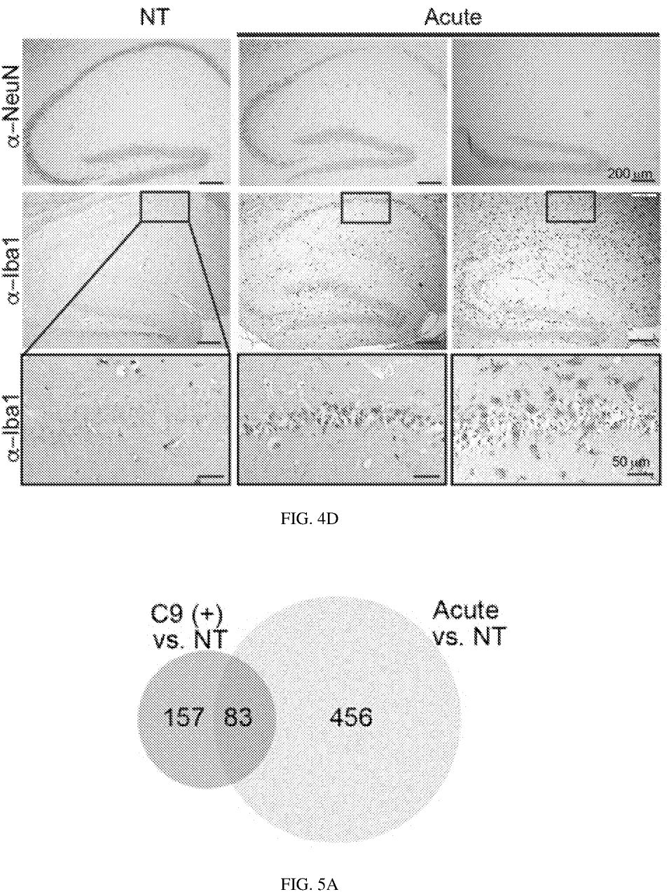

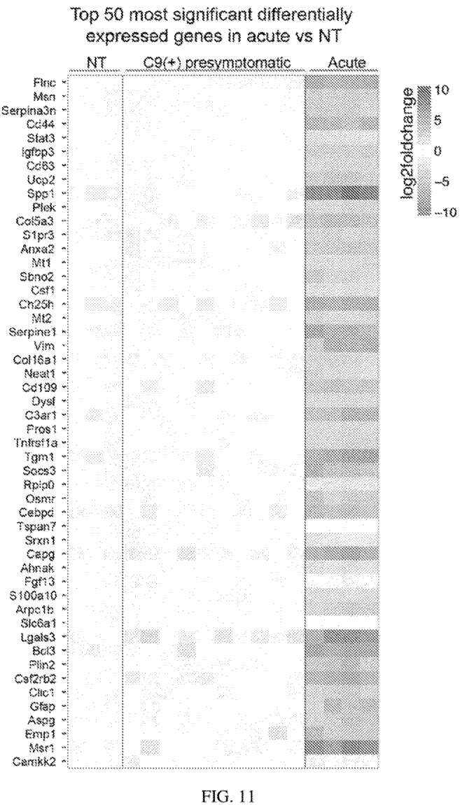

Sample to sample correlation based on differential gene expression measured using DeSeq2 shows that the acute cohort of C9-500 mice have unique gene expression profiles. Additionally, global gene expression changes in the NT animals are similar to the C9(+) animals, but acute animals were significantly different from both the NT and C9(+) pre-symptomatic animals (FIG. 4A). In the acute mice, 2,514 upregulated and 2,921 downregulated genes were observed, compared to NT animals, with a false discovery rate (FDR) < 0.05 (FIG. 4A). Heat maps of the top 50 significant genes in the acute vs. NT mice are shown in FIG. 11. Gene ontology (GO) analyses in the acute vs. NT mice show that the upregulated pathways include negative regulation of cell proliferation, inflammatory response, and actin cytoskeleton organization (p values indicated on the left) (FIG. 4B). Significantly downregulated pathways include brain development, synaptic organization, and neuron projection development (FIG. 4B).

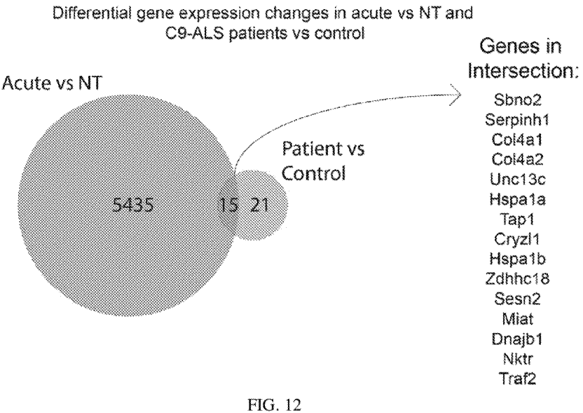

Since the acute C9-BAC mice mimic the neuropathology seen in end-stage C9-ALS patients, the gene expression profiles of the cortex from acute mice and C9-ALS patients were compared. RNA sequencing data obtained from a prior study was reanalyzed using STAR for alignment Kallisto to obtain transcript per million values. Using these parameters, 36 genes were identified that were consistently dysregulated between C9-ALS and unaffected individuals in this dataset. Of these, 15 of the 36 differentially expressed genes were also dysregulated in the acute mice, including Serpinhl (FIG. 12). This gene belongs to the serine protease inhibitor family, and several members of this gene family, including SerpinA3 and SerpinA1 are hypothesized to disrupt neuronal function and have been found to be differentially expressed in C9orf72 ALS patient autopsy tissue.

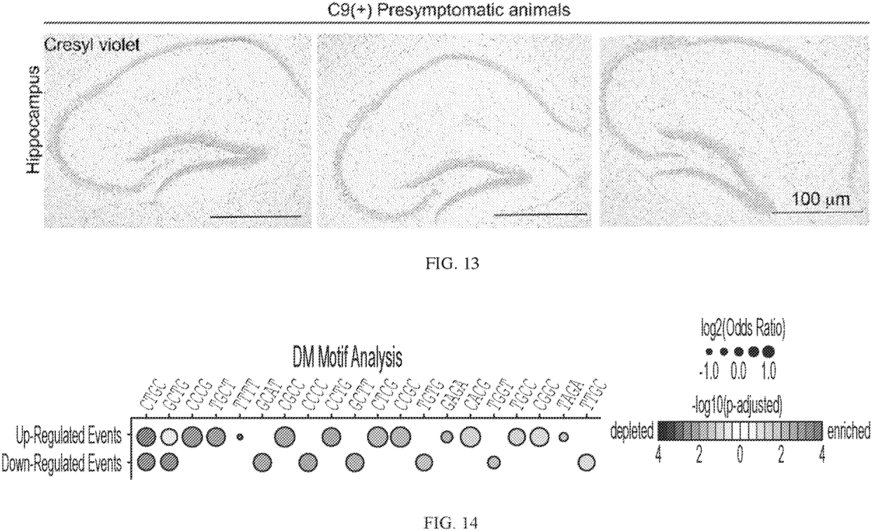

Since there were a large number of gene expression changes in the acute cohort that are likely changed in response to neuroinflammatory and neurodegenerative processes, the proportion of cell types present in each sample was estimated using a publicly available data set that shows the relative distribution of genes across seven different cell types-neurons, microglia, astrocytes, endothelial cells, oligodendrocyte precursor cells, myelinating oligodendrocytes and newly formed oligodendrocytes-in the mouse brain. Because there is considerable overlap between the expression profiles of oligodendrocyte precursor cells, myelinating oligodendrocytes and newly formed oligodendrocytes, these cell types were combined into a single category called “oligodendrocytes”. Using this data set as an index, an increase in the estimated proportion of microglial, oligodendrocyte and endothelial cells in acute vs NT mice, and a decrease in estimated proportion of neurons in Acute vs NT mice, were observed (FIG. 4C, Table 3). Additionally, significant, albeit small, differences were also observed between C9(+) pre-symptomatic and NT mice in the estimated proportion of endothelial, oligodendrocyte, and neuronal cells, but not in microglia (FIG. 4C, Table 3). Consistent with these findings, IHC shows overt loss of immunoreactivity to the neuronal marker NeuN and increased staining of the microglial marker Ibal in the acute mice compared to NT mice (FIG. 4D). Cresyl violet staining of C9(+) pre-symptomatic animals showed no overt pathology in the hippocampus (FIG. 13). No significant differences were observed in the estimated proportion of astrocytes in acute or C9(+) mice compared to NT (FIG. 4C).

Taken together, robust changes in gene expression were seen in acute mice compared to non-transgenic (NT) littermates. These changes are consistent with the neuronal loss and increased numbers of microglia in acute compared to NT mice. Similarly, pre-symptomatic C9(+) animals showed modest changes in cell type specific genes associated with neurons, endothelial cells and oligodendrocytes compared to NT mice.

### Table 3: Cell Type Analysis on Transcriptome Data From NT, C9(+) Pre-Symptomatic, and Acute Mice

Astrocytes

| | Samples | Mean 1 | Mean 2 | Statistics | P value | P adj |
|---|---|---|---|---|---|---|
| 1 | NT vs C9(+) | 0.037 | 0.035 | 0.651 | 0.5 | 1.000 |
| 2 | NT vs Acute | 0.037 | 0.034 | 3.575 | <0.0001 | 0.700 |
| 3 | C9(+) vs Acute | 0.035 | 0.034 | 4.256 | <0.0001 | 0.04 |

Endothelial cells

| | Samples | Mean 1 | Mean 2 | Statistics | P value | P adj |
|---|---|---|---|---|---|---|
| 1 | NT vs C9(+) | 0.075 | 0.084 | -13.735 | <0.0001 | <0.0001 |
| 2 | NT vs Acute | 0.075 | 0.106 | -31.664 | <0.0001 | <0.0001 |
| 3 | C9(+) vs Acute | 0.084 | 0.106 | -27.118 | <0.0001 | <0.0001 |

Microglia

| | Samples | Mean 1 | Mean 2 | Statistics | P value | P adj |
|---|---|---|---|---|---|---|
| 1 | NT vs C9(+) | 0.025 | 0.025 | -2.220 | 0.02 | 1 |
| 2 | NT vs Acute | 0.025 | 0.075 | -52.523 | <0.0001 | <0.0001 |
| 3 | C9(+) vs Acute | 0.025 | 0.075 | -53.598 | <0.0001 | <0.0001 |

Neurons

| | Samples | Mean 1 | Mean 2 | Statistics | P value | P adj |
|---|---|---|---|---|---|---|
| 1 | NT vs C9(+) | 0.68 | 0.65 | 25.62 | <0.0001 | <0.0001 |
| 2 | NT vs Acute | 0.68 | 0.52 | 53.21 | <0.0001 | <0.0001 |
| 3 | C9(+) vs Acute | 0.65 | 0.52 | 54.08 | <0.0001 | <0.0001 |

Oligodendrocytes

| | Samples | Mean 1 | Mean 2 | Statistics | P value | P adj |
|---|---|---|---|---|---|---|
| 1 | NT vs C9(+) | 0.186 | 0.207 | -18.742 | <0.0001 | <0.0001 |
| 2 | NT vs Acute | 0.186 | 0.269 | -46.016 | <0.0001 | <0.0001 |
| 3 | C9(+) vs Acute | 0.207 | 0.269 | -44.485 | <0.0001 | <0.0001 |

### Example 8: Abundant Alternative Splicing Changes Characterize Disease States in ALS/FTD

Changes in alternative splicing and alternative polyadenylation occur in C9-ALS, but not C9-negative sporadic, ALS cases. Alternative splicing changes in the mouse model were examined using MISO analyses to identify changes that occur during disease progression. Although few differentially expressed genes were detected during in pre-symptomatic animals, 240 and 539 genes showed alternative splicing changes in the C9(+) pre-symptomatic vs. NT, and acute vs. non-transgenic (NT) cohorts, respectively (FIG. 5A). These results demonstrate that alternative splicing abnormalities are an early molecular signature of C9orf72 ALS/FTD.

Eighty-three of these alternative splicing changes were shared between the acute and C9(+) pre-symptomatic cohorts (FIG. 5A), and the percent spliced in (hereinafter “psi”) values of these shared events increase with disease progression (FIG. 5B; “Intersection Events”). In contrast, psi values of the acute-only events are not altered in pre-symptomatic C9 mice (FIG. 5B; “Acute Only Events”). Elavl2, an RNA binding protein enriched in the neurons that affect neuronal excitability, was found to be alternatively spliced in both C9(+) pre-symptomatic animals and acute animals, and the psi value increased with disease progression (FIG. 5C). A summary of the alternatively spliced events found in both C9(+) pre-symptomatic and acute animals is shown in FIG. 5D. Because the psi values of these 83 genes are also changed in pre-symptomatic mice, and the psi values continue to increase with disease progression, these genes may be useful as biomarkers to monitor disease progression in C9-ALS/FTD patients (FIG. 5D, FIG. 11, Table 4).

GO analyses were used to better understand the categories of alternatively spliced genes that show changes in pre-symptomatic and acute mice, and changes common to pre-symptomatic and acutely affected animals (FIG. 5E). MISO was also performed to detect alternative splicing changes in previously published RNAseq data from human autopsy tissue (FIG. 5E). Splicing changes occurring in both acutely affected mice and end-stage C9-ALS patients were enriched for several similar GO categories, including genes with alternative splicing abnormalities that are normally involved in neuronal death, oxidative stress, cytoskeletal pathways, and inflammation. In contrast, pathways dysregulated in pre-symptomatic mice include synaptic transmission and membrane localization. FIG. 5F shows a motif analysis of alternative splicing events in pre-symptomatic C9(+) mice, acute C9(+) mice, and C9-ALS patients that demonstrates enrichment of motifs. In summary, it is shown herein that alternative splicing perturbations occur early in disease and progressively worsen with increase in disease severity.

TABLE 4

Common alternative splicing events in C9(+) pre-symptomatic and Acute mice

| | | | NT | | |
|---|---|---|---|---|---|
| #Event | gene | symb | 638 mean | 645 mean | 1196 mean |
| chr2:38514581:38514658:+@chr2:38514733:38514864:+@chr2:38550553:38550671:+ | NM_021606 | Nek6 | 0.63 | 0.49 | 0.58 |
| chr15:93410692:93410783:+@chr15:93420284:93420490:+@chr15:93424014:93424075:+ | NM_001285864 | Pphln1 | 0.32 | 0.51 | 0.44 |
| chr3:81036416:81037554:+@chr3:81131018:81131076:+@chr3:81141405:81141600:+ | NM_019971 | Pdgfc | 0.51 | 0.64 | 0.6 |
| chr2:32244234:32244389:+@chr2:32244786:32244950:+@chr2:32245547:32245677:+ | NM_145145 | Pomt1 | 0.57 | 0.56 | 0.6 |
| chr19:40344356:40344439:-@chr19:40340045:40340146:-@chr19:40336986:40337035:- | NM_001034964 | Sorbs 1 | 0.84 | 0.84 | 0.85 |
| chr2:79657147:79658349:+@chr2:79658670:79658846:+@chr2:79660233:79660310:+ | NM_080558 | Ssfa2 | 0.38 | 0.52 | 0.49 |
| chr19:4161068:4161215:-@chr19:4159566:4159705:-@chr19:4159403:4159460:- | NM_021485 | Rps6kb2 | 0.73 | 0.7 | 0.77 |
| chrX:74018130:74018366:-@chrX:74017901:74018017:-@chrX:74017130:74017511:- | NM_001177976 | Irak1 | 0.31 | 0.27 | 0.23 |
| chrll:31908825:31908875:+@chrll:31918801:31918824:+@chrll:31920409:31920582:+ | NM_001290676 | Cpeb4 | 0.73 | 0.82 | 0.73 |
| chr6:29361294:29361487:+@chr6:29361560:29361753:+@chr6:29366040:29366206:+ | NM_001285412 | Calu | 0.73 | 0.67 | 0.62 |
| chr5:5556065:5556156:-@chr5:5555238:5555406:-@chr5:5546353:5546405:- | NM_153116 | Gtpbp 10 | 0.6 | 0.58 | 0.49 |
| chr8:127409556:127409707:+@chr8:127410729:127410773:+@chr8:127415570:127415797:+ | NM_033620 | Pard3 | 0.7 | 0.73 | 0.67 |
| chr17:56686006:56686061:+@chr17:56692521:56692721:+@chr17:56696602:56696638:+ | NM_027933 | Ranbp3 | 0.42 | 0.45 | 0.35 |
| chr7:27507947:27508103:+@chr7:27515251:27515447:+@chr7:27516040:27520041:+ | NM_019412 | Prx | 0.59 | 0.73 | 0.73 |
| chr6:112644588:112644747:-@chr6:112628462:112628524:-@chr6:112619851:112620904:- | NM_001167730 | Rad18 | 0.69 | 0.77 | 0.6 |
| chr6:149310322:149310377:+@chr6:149324732:149324800:+@chr6:149325405:149329784:+ | NM_026054 | 2810474 O19Rik | 0.33 | 0.51 | 0.4 |
| chr15:34117880:34118110:+@chr15:34121423:34121512:+@chr15:34123668:34123766:+ | NM_026002 | Mtdh | 0.31 | 0.33 | 0.49 |
| chr2:124298575:124298655:+@chr2:124400243:124400365:+@chr2:124489985:124490012:+ | NM_001290997 | Sema6d | 0.14 | 0.13 | 0.05 |
| chr16:59618795:59618850:-@chr16:59618666:59618678:-@chrl6:59613321:59613844:- | NM_019665 | Arl6 | 0.28 | 0.42 | 0.27 |
| chr17:15499888:15500024:+@chr17:15501154:15501202:+@chr17:15502962:15503128:+ | NM_013684 | Tbp | 0.52 | 0.52 | 0.6 |
| chr16:3984991:3987176:-@chr16:3982979:3983242:-@chr16:3981585:3981687:- | NM_177472 | Slx4 | 0.25 | 0.3 | 0.24 |
| chr2:22957018:22957118:-@chr2:22950184:22950270:-@ chr2:22946576:22946761:- | NM_001077190 | Abi1 | 0.16 | 0.24 | 0.27 |
| chr18:12862107:12862713:-@chr18:12841184:12841346:-@chrl8:12819505:12819661:- | NM_001252492 | Osbp11a | 0.85 | 0.88 | 0.87 |
| chr10: 19904563: 19904713:-@chr10:19894725:19894802:-@chr10:19894250:19894358:- | NM_001161825 | Pex7 | 0.85 | 0.88 | 0.91 |
| chr1:193362013:193362133:-@chr1:193361580: 193361667:-@chr1:193359719:193359847:- | NM_144817 | Camk1g | 0.15 | 0.4 | 0.12 |
| chr13:58160152:58160238:+@chr13:58162838:58162881:+@chr13:58163433:58164693:+ | NM_198004 | Idnk | 0.27 | 0.3 | 0.15 |
| chr8:69744461:69744587:-@chr8:69744192:69744252:-@chr8:69742859:69743269:- | NM_001200023 | Zfp963 | 0.35 | 0.32 | 0.34 |
| chrX:50573224:50573351:-@chrX:50573015:50573141:-@chrX:50569277:50570291:- | NR_015505 | Firre | 0.53 | 0.55 | 0.47 |
| chr5:5557213:5557406:-@chr5:5555238:5555382:-@chr5:5547927:5548000:- | NM_153116 | Gtpbp10 | 0.64 | 0.55 | 0.59 |
| chr16:87914154:87914404:-@chr16:87896809:87896895:-@chrl6:87895897:87896351:- | NM_010348 | Grikl | 0.64 | 0.49 | 0.48 |
| chr14:47704187:47704277:+@chr14:47704420:47704488:+@chr14:47705763:47705800:+ | NM_001293636 | Ktn1 | 0.06 | 0.13 | 0.15 |
| chr10:14760289:14760710:+@chr10:14766677:14767025:+@chr10:14770155:14770556:+ | NM_008703 | Nmbr | 0.89 | 0.76 | 0.83 |
| chr11:4118625:4118729:-@chr11:4116681:4116756:-@chrl1:4111894:4111937:- | NM_144520 | Sec1412 | 0.66 | 0.65 | 0.77 |
| chr19:4811172:4811634:-@chr19:4793926:4794009:-@chr19:4792398:4792820:- | NM_001290127 | Gm21992 | 0.31 | 0.34 | 0.32 |
| chr17:56011866:56011962:+@chr17:56012271:56012492:+@chr17:56014941:56015109:+ | NM_026530 | Mpnd | 0.58 | 0.82 | 0.82 |
| chr4:91260761:91260986:-@chr4:91254141:91254179:-@chr4:91250767:91253570:- | NM_001177883 | Elavl2 | 0.64 | 0.71 | 0.76 |
| chrX:71376887:71376910:+@chrX:71379014:71379064:+@chrX:71383200:71383275:+ | NM_016985 | Mtmr1 | 0.2 | 0.26 | 0.22 |
| chr17:85615594:85615743:-@chr17:85614648:85614727:-@chr17:85609090:85611748:- | NR_015387 | Six3os1 | 0.66 | 0.75 | 0.58 |
| chr7:96694770:96695025:+@ chr7:96702642:96702740:+@ chr7:96703926:96704161:+ | NM_011858 | Tenm4 | 0.36 | 0.3 | 0.38 |
| chr6:42350946:42351148:+@chr6:42351270:42351362:+@chr6:42351475:42351969:+ | NM_001289618 | Zyx | 0.33 | 0.49 | 0.44 |
| chr8:3606267:3606406:+@chr8:3606504:3606820:+@chr8:3607791:3607890:+ | NM_001163749 | Camsap3 | 0.66 | 0.77 | 0.54 |
| chr5:140713053:140713172:+@chr5:140714293:140714384:+@chr5:140714567:140714685:+ | NM_001276287 | Brat1 | 0.49 | 0.52 | 0.6 |
| chr11:102224734:102224955:-@chr11:102219145:102219281:-@chr11:102218416:102218781:- | NM_001284249 | Hdac5 | 0.17 | 0.25 | 0.25 |
| chr15:99319131:99319254:-@chr15:99318918:99319017:-@chr15:99317223:99318372:- | NM_011711 | Fmn13 | 0.54 | 0.54 | 0.55 |
| chr2:124298575:124298655:+@chr2:124400243:124400364:+@chr2:124489985:124490012:+ | NM_001290997 | Sema6d | 0.16 | 0.04 | 0.05 |
| chr5:32785549:32785626:-@chr5:32774396:32774466:-@chr5:32764773:32764948:- | NM_177298 | Pisd | 0.86 | 0.86 | 0.9 |
| chrX:60295189:60295292:-@chrX:60293607:60293657:-@chrX:60292319:60292383:- | NM_001037863 | Atp11c | 0.67 | 0.53 | 0.48 |
| chr7:4010466:4010503:-@chr7:4010251:4010300:-@chr7:4007073:4009834:- | NM_001302675 | Lair1 | 0.49 | 0.37 | 0.55 |
| chr4:21746656:21746774:+@chr4:21747483:21747576:+@chr4:21748137:21750546:+ | NM_001290420 | Ccnc | 0.3 | 0.33 | 0.31 |
| chr2:157560448:157560535:+@chr2:157561213:157561293:+@chr2:157561542:157562519:+ | NM_010923 | Nnat | 0.33 | 0.28 | 0.31 |
| chr19:12577090:12577170:+@chr19:12583923:12584122:+@chr19:12586835:12589696:+ | NM_026640 | Fam111a | 0.5 | 0.52 | 0.45 |
| chr17:94747617:94748417:-@chr17:94743932:94743994:-@chr17:94740386:94740755:- | NM_176917 | Mett14 | 0.39 | 0.42 | 0.37 |
| chr12:101082451:101084025:+@chr12:101084284:101084499:+@chr12:101088042:101088927:+ | NR_038049 | D130020 L05Rik | 0.77 | 0.79 | 0.81 |
| chr6:145211170:145211244:+@chr6:145214522:145214784:+@chr6:145215184:145215271:+ | NM_001163628 | Etfrf1 | 0.27 | 0.33 | 0.43 |
| chr7:73514090:73514151:-@chr7:73507806:73507913:-@chr7:73504988:73505128:- | NM_001081345 | Chd2 | 0.65 | 0.79 | 0.61 |
| chr10:128645721:128645993:-@chr10:128643875:128643968:-@chr10:128642629:128642733:- | NM_011772 | Ikzf4 | 0.63 | 0.78 | 0.82 |
| chr8:85841002:85841103:+@chr8:85843021:85843112:+@chr8:85875634:85875723:+ | NM_199446 | Phkb | 0.62 | 0.63 | 0.67 |
| chr18:59062381:59062562:+@chr18:59065835:59065885:+@chr18:59072193:59072417:+ | NR_033765 | A730017 C20Rik | 0.66 | 0.76 | 0.85 |
| chr7:123179717:123179859:+@chr7:123180249:123180395:+@chr7:123180540:123180677:+ | NM_144925 | Tnrc6a | 0.56 | 0.69 | 0.78 |
| chr11:95046921:95047046:-@chr11:95046449:95046590:-@chrl 1:95044482:95045763:- | NM_00130607 1 | Itga3 | 0.26 | 0.14 | 0.27 |
| chrX: 135751945:135752017:+@chrX: 135796142:135796336:+@chrX: 13 5797553:135797629:+ | NM_026081 | Gprasp1 | 0.41 | 0.44 | 0.42 |
| chr4:118234959:118235103:- @ chr4:118231486:118231791:-@ chr4:118230695:1182308 8 8: - | NM_011213 | Ptprf | 0.16 | 0.2 | 0.2 |
| chr16:45408856:45409053:- @ chr16:45400222:45400303:-@chr16:45396969:45397295:- | NM_010818 | Cd200 | 0.93 | 0.95 | 0.95 |
| chr2:71228600:71228690:+@ chr2:71233649:71233708:+@ chr2:71235915:71236030:+ | NM_010064 | Dyncli2 | 0.86 | 0.9 | 0.86 |
| chrX: 151835224:151835292:+@chrX: 151836222:151836329:+@chrX: 15 1840056:151840154:+ | NM_021523 | Huwel | 0.27 | 0.24 | 0.26 |
| chr9:31157695:31157847:-@chr9:31156190:31156225:-@ chr9:3 1153414:3 1153499: - | NM_00 110245 5 | Aplp2 | 0.86 | 0.88 | 0.84 |
| chr3:146458743:146458840:+@ chr3:146459679:146459840:+@ chr3:146 463899:146465771:+ | NM_00129367 2 | Ctbs | 0.76 | 0.78 | 0.77 |
| chr 11: 70609166:70609254: + @ chr 11: 70609408:7060943 1: + @ chr 11: 7060 9554:70609717:+ | NM_016713 | Minkl | 0.93 | 0.96 | 0.95 |
| chr4:11287475:11287633:-@chr4:11285078:11285173:-@ chr4:1128 1004:1128 1124: - | NM_00108120 1 | Dpyl914 | 0.88 | 0.9 | 0.89 |
| chr11:28434081:28434157:-@chr11:28396046:28396165:-@chrl 1:28385692:28388948:- | NM_00116666 1 | Ccdc85a | 0.75 | 0.74 | 0.61 |
| chr4:9635901:9635969: - @ chr4:9624360:962440 1: -@ chr4:9610843:961093 8:- | NM_023066 | Asph | 0.16 | 0.22 | 0.21 |
| chr8:77155202:77155318:+@ chr8:77187520:77187664:+@ chr8:7721064 4:77210774:+ | NM_00108390 6 | Nr3c2 | 0.87 | 0.91 | 0.91 |
| chr13: 107022012: 107022114:-@chr13:106996747:106997129:-@chrl3:106993848:106993942:- | NM_00114577 9 | Kif2a | 0.1 | 0.04 | 0.04 |
| chr7:109960074:109960422:-@chr7:109935482:109935553:-@ chr7:109934693:109934802:- | NM_021494 | Dennd5a | 0.46 | 0.48 | 0.45 |
| chr10:81314992:81315118:+@chr10:81317326:81317403:+@chr10:8131 | NM_00114668 | Pip5klc | 0.8 | 0.75 | 0.82 |
| 7766:81319974:+ | 7 | | | | |
| chrX:74337911:74338054:+@chrX:74338134:74338368:+@chrX:743384 59:74338601:+ | NM_008883 | Plxna3 | 0.74 | 0.79 | 0.76 |
| chrX:95166424:95166486:-@chrX:95165593:95165970:@chrX:95130836:95131015:- | NM_00129038 5 | Arhgef9 | 0.96 | 0.96 | 0.96 |
| chrX:152178964:152179090:+@chrX:152180997:152181092:+@chrX: 15 2202908:152203169:+ | NM_00111466 4 | Iqsec2 | 0.07 | 0.1 | 0.04 |
| chr16:32898878:32898939:+@chr16:32900633:32900707:+@chr16:3290 2419:32902545:+ | NM_00115934 9 | Fyttd1 | 0.92 | 0.93 | 0.96 |
| chr11:75349669:75349814:+@chr11:75382250:75382394:+@chr11:7538 7411:75387500:+ | NM_00110261 1 | Smyd4 | 0.83 | 0.83 | 0.79 |
| chr4:129600983:129601134:+@chr4:129601407:129601533:+@chr4:129 602195:129602261:+ | NM_029748 | Tmem23 4 | 0.27 | 0.25 | 0.23 |
| chr2:31950529:31950590:+@chr2:31962215:31962281:+@chr2:3196963 0:31969792:+ | NM_145144 | Aifll | 0.72 | 0.77 | 0.72 |
| chr5:81771584:81771680:+@chr5:81792361:81792412:+@chr5:8179426 4:81795347:+ | NM_198702 | Adgrl3 | 0.13 | 0.09 | 0.17 |

TABLE 4, continued.

| | | | Pre-symptomatic C9 | | | | | | | | | |
|---|---|---|---|---|---|---|---|---|---|---|---|---|
| #Event | gene | symb | 620_ mea | 621_ mea | 636_ mea | 637_ mea | 644_ mea | 646_ mea | 648_ mea | 660_ mea | 668_ mea | 673_ mea |
| | | | n | n | n | n | n | n | n | n | n | n |
| chr2:38514581:38514658:+@chr2:38 514733:38514864:+@chr2:38550553: 38550671:+ | NM_02 1606 | Nek6 | 0.41 | 0.31 | 0.49 | 0.46 | 0.44 | 0.34 | 0.43 | 0.29 | 0.35 | 0.55 |
| chr15:93410692:93410783:+@chr15: 93420284:93420490:+@chr15:934240 14:93424075:+ | NM_00 128586 4 | Pphln1 | 0.13 | 0.13 | 0.07 | 0.11 | 0.24 | 0.19 | 0.38 | 0.11 | 0.21 | 0.17 |
| chr3:81036416:81037554:+@chr3:81 131018:81131076:+@chr3:81141405: 81141600:+ | NM_01 9971 | Pdgfc | 0.38 | 0.46 | 0.29 | 0.44 | 0.34 | 0.46 | 0.26 | 0.34 | 0.17 | 0.23 |
| chr2:32244234:32244389:+@chr2:32 244786:32244950:+@chr2:32245547: 32245677:+ | NM_14 5145 | Pomt1 | 0.41 | 0.18 | 0.24 | 0.33 | 0.56 | 0.53 | 0.53 | 0.24 | 0.38 | 0.24 |
| chr19:40344356:40344439:-@chr19:40340045:40340146:-@chr19:40336986:40337035:- | NM_00 103496 4 | Sorbs1 | 0.83 | 0.75 | 0.69 | 0.62 | 0.82 | 0.68 | 0.74 | 0.75 | 0.76 | 0.75 |
| chr2:79657147:79658349:+@chr2:79 658670:79658846:+@chr2:79660233: 79660310:+ | NM_08 0558 | Ssfa2 | 0.31 | 0.31 | 0.29 | 0.34 | 0.19 | 0.45 | 0.31 | 0.3 | 0.22 | 0.37 |
| chr19:4161068:4161215:-@chr19:4159566:4159705:-@chr19:4159403:4159460:- | NM_02 1485 | Rps6k b2 | 0.63 | 0.58 | 0.58 | 0.63 | 0.47 | 0.57 | 0.6 | 0.66 | 0.51 | 0.54 |
| chrX:74018130:74018366:-@chrX:74017901:74018017:-@chrX:74017130:74017511:- | NM_00 117797 6 | Irak1 | 0.07 | 0.06 | 0.12 | 0.08 | 0.14 | 0.07 | 0.14 | 0.27 | 0.09 | 0.12 |
| chr11:31908825:31908875:+@chr11: 31918801:31918824:+@chr11:319204 09:31920582:+ | NM_00 129067 6 | Cpeb4 | 0.8 | 0.53 | 0.6 | 0.67 | 0.71 | 0.68 | 0.47 | 0.3 | 0.56 | 0.72 |
| chr6:29361294:29361487:+@chr6:29 361560:29361753:+@chr6:29366040: 29366206:+ | NM_00 128541 2 | Calu | 0.49 | 0.52 | 0.49 | 0.41 | 0.41 | 0.63 | 0.44 | 0.45 | 0.43 | 0.39 |
| chr5:5556065:5556156:-@chr5:5555238:5555406:-@chr5:5546353:5546405:- | NM_15 3116 | Gtpbp 10 | 0.56 | 0.74 | 0.69 | 0.64 | 0.7 | 0.67 | 0.79 | 0.62 | 0.63 | 0.75 |
| chr8:127409556:127409707:+@chr8: 127410729:127410773:+@chr8:12741 5570:127415797:+ | NM_03 3620 | Pard3 | 0.58 | 0.63 | 0.41 | 0.56 | 0.51 | 0.48 | 0.41 | 0.49 | 0.64 | 0.44 |
| chr17:56686006:56686061:+@chr17: 56692521:56692721:+@chr17:566966 02:56696638:+ | NM_02 7933 | Ranbp 3 | 0.23 | 0.19 | 0.22 | 0.09 | 0.21 | 0.45 | 0.18 | 0.23 | 0.11 | 0.14 |
| chr7:27507947:27508103:+@chr7:27 515251:27515447:+@chr7:27516040: 27520041:+ | NM_01 9412 | Prx | 0.72 | 0.34 | 0.49 | 0.44 | 0.58 | 0.43 | 0.38 | 0.56 | 0.33 | 0.63 |
| chr6:112644588:112644747:- | NM_00 | Rad18 | 0.6 | 0.33 | 0.64 | 0.33 | 0.54 | 0.67 | 0.39 | 0.5 | 0.29 | 0.56 |
| @chr6:112628462:112628524:-@chr6:112619851:112620904:- | 116773 0 | | | | | | | | | | | |
| chr6:149310322:149310377:+@chr6: 149324732:149324800:+@chr6:14932 5405:149329784:+ | NM_02 6054 | 28104 74019 Rik | 0.28 | 0.47 | 0.22 | 0.15 | 0.28 | 0.15 | 0.19 | 0.35 | 0.21 | 0.29 |
| chr15:34117880:34118110:+@chr15: 34121423:34121512:+@chr15:341236 68:34123766:+ | NM_02 6002 | Mtdh | 0.32 | 0.26 | 0.3 | 0.26 | 0.29 | 0.18 | 0.22 | 0.3 | 0.19 | 0.19 |
| chr2:124298575:124298655:+@chr2: 124400243:124400365:+@chr2:12448 9985:124490012:+ | NM_00 129099 7 | Sema6 d | 0.24 | 0.25 | 0.38 | 0.12 | 0.17 | 0.26 | 0.15 | 0.33 | 0.19 | 0.22 |
| chr16:59618795:59618850:-@chr16:59618666:59618678:-@chr16:59613321:59613844:- | NM_01 9665 | Arl6 | 0.12 | 0.29 | 0.13 | 0.11 | 0.2 | 0.19 | 0.1 | 0.07 | 0.14 | 0.18 |
| chr17:15499888:15500024:+@chr17: 15501154:15501202:+@chr17:155029 62:15503128:+ | NM_01 3684 | Tbp | 0.41 | 0.43 | 0.35 | 0.23 | 0.34 | 0.3 | 0.26 | 0.4 | 0.54 | 0.49 |
| chr16:3984991:3987176:-@chr16:3982979:3983242:-@chr16:3981585:3981687:- | NM_17 7472 | Slx4 | 0.1 | 0.08 | 0.05 | 0.04 | 0.21 | 0.11 | 0.2 | 0.24 | 0.06 | 0.17 |
| chr2:22957018:22957118:-@chr2:22950184:22950270:- | NM_00 107719 | Abil | 0.59 | 0.39 | 0.3 | 0.41 | 0.4 | 0.55 | 0.32 | 0.23 | 0.45 | 0.28 |
| @chr2:22946576:22946761:- | 0 | | | | | | | | | | | |
| chr18:12862107:12862713:-@chr18:12841184:12841346:-@chr18:12819505:12819661:- | NM_00 125249 2 | Osbpl 1a | 0.74 | 0.68 | 0.83 | 0.76 | 0.85 | 0.72 | 0.8 | 0.76 | 0.76 | 0.65 |
| chr10:19904563:19904713:-@chr10:19894725:19894802:-@chr10:19894250:19894358:- | NM_00 116182 5 | Pex7 | 0.68 | 0.65 | 0.7 | 0.74 | 0.88 | 0.84 | 0.86 | 0.8 | 0.79 | 0.83 |
| chr1:193362013:193362133:-@chr1:193361580:193361667:-@chr1:193359719:193359847:- | NM_14 4817 | Camk 1g | 0.07 | 0.06 | 0.1 | 0.05 | 0.06 | 0.08 | 0.15 | 0.04 | 0.1 | 0.11 |
| chr13:58160152:58160238:+@chr13: 58162838:58162881:+@chr13:581634 33:58164693:+ | NM_19 8004 | Idnk | 0.17 | 0.1 | 0.15 | 0.06 | 0.11 | 0.15 | 0.11 | 0.13 | 0.15 | 0.14 |
| chr8:69744461:69744587:-@chr8:69744192:69744252:-@chr8:69742859:69743269:- | NM_00 120002 3 | Zfp96 3 | 0.35 | 0.63 | 0.55 | 0.35 | 0.36 | 0.43 | 0.38 | 0.6 | 0.39 | 0.43 |
| chrX:50573224:50573351:-@chrX:50573015:50573141:-@chrX:50569277:50570291:- | NR_01 5505 | Firre | 0.66 | 0.68 | 0.55 | 0.83 | 0.73 | 0.62 | 0.55 | 0.68 | 0.59 | 0.61 |
| chr5:5557213:5557406:-@chr5:5555238:5555382:-@ chr5:5547927:5548000:- | NM_15 3116 | Gtpbp 10 | 0.81 | 0.67 | 0.67 | 0.65 | 0.8 | 0.62 | 0.72 | 0.56 | 0.71 | 0.79 |
| chr16:87914154:87914404:-@chr16:87896809:87896895:-@chr16:87895897:87896351:- | NM_01 0348 | Grik1 | 0.15 | 0.17 | 0.46 | 0.19 | 0.28 | 0.41 | 0.35 | 0.47 | 0.52 | 0.33 |
| chr14:47704187:47704277:+@chr14: 47704420:47704488:+@chr14:477057 63:47705800:+ | NM_00 129363 6 | Ktn1 | 0.16 | 0.16 | 0.22 | 0.23 | 0.17 | 0.38 | 0.11 | 0.19 | 0.33 | 0.19 |
| chr10:14760289:14760710:+@chr10: 14766677:14767025:+@chr10:147701 55:14770556:+ | NM_00 8703 | Nmbr | 0.76 | 0.74 | 0.69 | 0.59 | 0.77 | 0.68 | 0.6 | 0.48 | 0.62 | 0.66 |
| chr11:4118625:4118729:-@chr11:4116681:4116756:-@chr11:4111894:4111937:- | NM_14 4520 | Sec141 2 | 0.85 | 0.85 | 0.82 | 0.82 | 0.85 | 0.79 | 0.74 | 0.74 | 0.85 | 0.78 |
| chr19:4811172:4811634:-@chr19:4793926:4794009:-@chr19:4792398:4792820:- | NM_00 129012 7 | Gm21 992 | 0.48 | 0.43 | 0.31 | 0.5 | 0.61 | 0.52 | 0.63 | 0.39 | 0.38 | 0.34 |
| chr17:56011866:56011962:+@chr17: 56012271:56012492:+@chr17:560149 41:56015109:+ | NM_02 6530 | Mpnd | 0.94 | 0.87 | 0.77 | 0.94 | 0.87 | 0.86 | 0.88 | 0.94 | 0.92 | 0.86 |
| chr4:91260761:91260986:-@chr4:91254141:91254179:-@chr4:91250767:91253570:- | NM_00 117788 3 | Elavl2 | 0.92 | 0.83 | 0.79 | 0.89 | 0.74 | 0.78 | 0.84 | 0.89 | 0.73 | 0.81 |
| chrX:71376887:71376910:+@chrX:71 | NM_01 | Mtmr1 | 0.31 | 0.62 | 0.48 | 0.5 | 0.38 | 0.49 | 0.21 | 0.3 | 0.44 | 0.42 |
| 379014:71379064:+@chrX:71383200: 71383275:+ | 6985 | | | | | | | | | | | |
| chr17:85615594:85615743:-@chr17:85614648:85614727:-@chr17:85609090:85611748:- | NR_01 5387 | Six3os 1 | 0.38 | 0.27 | 0.41 | 0.43 | 0.32 | 0.44 | 0.35 | 0.58 | 0.51 | 0.47 |
| chr7:96694770:96695025:+@chr7:96 702642:96702740:+@chr7:96703926: 96704161:+ | NM_01 1858 | Tenm4 | 0.57 | 0.5 | 0.43 | 0.58 | 0.7 | 0.6 | 0.4 | 0.59 | 0.56 | 0.41 |
| chr6:42350946:42351148:+@chr6:42 351270:42351362:+@chr6:42351475: 42351969:+ | NM_00 128961 8 | Zyx | 0.76 | 0.56 | 0.56 | 0.52 | 0.55 | 0.79 | 0.59 | 0.67 | 0.72 | 0.52 |
| chr8:3606267:3606406:+@chr8:3606 504:3606820:+@chr8:3607791:36078 90:+ | NM_00 116374 9 | Camsa p3 | 0.85 | 0.88 | 0.79 | 0.9 | 0.87 | 0.71 | 0.88 | 0.77 | 0.74 | 0.85 |
| chr5:140713053:140713172:+@chr5: 140714293:140714384:+@chr5:14071 4567:140714685:+ | NM_00 127628 7 | Brat1 | 0.72 | 0.79 | 0.67 | 0.75 | 0.76 | 0.73 | 0.85 | 0.84 | 0.76 | 0.81 |
| chr11:102224734:102224955:-@chr11:102219145:102219281:-@chr11:102218416:102218781:- | NM_00 128424 9 | Hdac5 | 0.26 | 0.39 | 0.44 | 0.45 | 0.41 | 0.51 | 0.37 | 0.47 | 0.49 | 0.24 |
| chr15:99319131:99319254:-@chr15:99318918:99319017:- | NM_01 1711 | Fmnl3 | 0.72 | 0.75 | 0.68 | 0.72 | 0.6 | 0.49 | 0.75 | 0.56 | 0.81 | 0.76 |
| @chr15:99317223:99318372:- | | | | | | | | | | | | |
| chr2:124298575:124298655:+@chr2: 124400243:124400364:+@chr2:12448 9985:124490012:+ | NM_00 129099 7 | Sema6 d | 0.33 | 0.23 | 0.54 | 0.09 | 0.26 | 0.18 | 0.16 | 0.34 | 0.18 | 0.34 |
| chr5:32785549:32785626:-@chr5:32774396:32774466:-@chr5:32764773:32764948:- | NM_17 7298 | Pisd | 0.92 | 0.85 | 0.82 | 0.8 | 0.79 | 0.59 | 0.8 | 0.51 | 0.55 | 0.41 |
| chrX:60295189:60295292:-@chrX:60293607:60293657:-@chrX:60292319:60292383:- | NM_00 103786 3 | Atp11 c | 0.77 | 0.77 | 0.82 | 0.61 | 0.71 | 0.75 | 0.78 | 0.51 | 0.8 | 0.75 |
| chr7:4010466:4010503:-@chr7:4010251:4010300:-@chr7:4007073:4009834:- | NM_00 130267 5 | Lair1 | 0.65 | 0.71 | 0.48 | 0.56 | 0.58 | 0.65 | 0.66 | 0.7 | 0.62 | 0.55 |
| chr4:21746656:21746774:+@chr4:21 747483:21747576:+@chr4:21748137: 21750546:+ | NM_00 129042 0 | Ccnc | 0.47 | 0.48 | 0.55 | 0.53 | 0.49 | 0.32 | 0.67 | 0.58 | 0.6 | 0.56 |
| chr2:157560448:157560535:+@chr2: 157561213:157561293:+@chr2:15756 1542:157562519:+ | NM_01 0923 | Nnat | 0.65 | 0.52 | 0.32 | 0.6 | 0.42 | 0.51 | 0.35 | 0.49 | 0.56 | 0.38 |
| chr19:12577090:12577170:+@chr19: 12583923:12584122:+@chr19:125868 35:12589696:+ | NM_02 6640 | Fam11 1a | 0.74 | 0.77 | 0.75 | 0.76 | 0.75 | 0.61 | 0.41 | 0.71 | 0.57 | 0.56 |
| chr17:94747617:94748417:-@chr17:94743932:94743994:-@chr17:94740386:94740755:- | NM_17 6917 | Mettl4 | 0.78 | 0.49 | 0.78 | 0.59 | 0.84 | 0.54 | 0.5 | 0.65 | 0.5 | 0.69 |
| chr12:101082451:101084025:+@chr1 2:101084284:101084499:+@chr12:10 1088042:101088927:+ | NR_03 8049 | D1300 20L05 Rik | 0.17 | 0.76 | 0.48 | 0.53 | 0.47 | 0.4 | 0.38 | 0.42 | 0.55 | 0.74 |
| chr6:145211170:145211244:+@chr6: 145214522:145214784:+@chr6:14521 5184:145215271:+ | NM_00 116362 8 | Etfrf1 | 0.81 | 0.39 | 0.51 | 0.35 | 0.69 | 0.69 | 0.48 | 0.8 | 0.63 | 0.65 |
| chr7:73514090:73514151:-@chr7:73507806:73507913:-@chr7:73504988:73505128:- | NM_00 108134 5 | Chd2 | 0.43 | 0.6 | 0.32 | 0.57 | 0.51 | 0.56 | 0.56 | 0.57 | 0.69 | 0.57 |
| chr10:128645721:128645993:-@chr10:128643875:128643968:-@chr10:128642629:128642733:- | NM_01 1772 | Ikzf4 | 0.42 | 0.54 | 0.73 | 0.69 | 0.73 | 0.61 | 0.61 | 0.43 | 0.48 | 0.59 |
| chr8:85841002:85841103:+@chr8:85 843021:85843112:+@chr8:85875634: 85875723:+ | NM_19 9446 | Phkb | 0.58 | 0.63 | 0.5 | 0.44 | 0.2 | 0.53 | 0.5 | 0.55 | 0.38 | 0.53 |
| chr18:59062381:59062562:+@chr18: 59065835:59065885:+@chr18:590721 93:59072417:+ | NR_03 3765 | A7300 17C20 Rik | 0.36 | 0.65 | 0.58 | 0.46 | 0.7 | 0.63 | 0.76 | 0.49 | 0.32 | 0.65 |
| chr7:123179717:123179859:+@chr7: | NM_14 | Tnrc6a | 0.76 | 0.43 | 0.42 | 0.44 | 0.68 | 0.33 | 0.29 | 0.48 | 0.5 | 0.51 |
| 123180249:123180395:+@chr7:12318 0540:123180677:+ | 4925 | | | | | | | | | | | |
| chr11:95046921:95047046:-@chr11:95046449:95046590:-@chr11:95044482:95045763:- | NM_00 130607 1 | Itga3 | 0.43 | 0.59 | 0.4 | 0.18 | 0.69 | 0.59 | 0.6 | 0.28 | 0.34 | 0.35 |
| chrX:135751945:135752017:+@chrX: 135796142:135796336:+@chrX: 1357 97553:135797629:+ | NM_02 6081 | Gpras p1 | 0.21 | 0.35 | 0.33 | 0.29 | 0.32 | 0.35 | 0.5 | 0.33 | 0.4 | 0.36 |
| chr4:118234959:118235103:-@chr4:118231486:118231791:-@chr4:118230695:118230888:- | NM_01 1213 | Ptprf | 0.29 | 0.28 | 0.15 | 0.25 | 0.21 | 0.24 | 0.24 | 0.23 | 0.31 | 0.21 |
| chr16:45408856:45409053:-@chr16:45400222:45400303:-@chr16:45396969:45397295:- | NM_01 0818 | Cd200 | 0.92 | 0.94 | 0.86 | 0.93 | 0.91 | 0.86 | 0.91 | 0.88 | 0.9 | 0.91 |
| chr2:71228600:71228690:+@chr2:71 233649:71233708:+@chr2:71235915: 71236030:+ | NM_01 0064 | Dync1 i2 | 0.85 | 0.78 | 0.77 | 0.81 | 0.83 | 0.82 | 0.77 | 0.83 | 0.9 | 0.79 |
| chrX:151835224:151835292:+@chrX: 151836222:151836329:+@chrX:1518 40056:151840154:+ | NM_02 1523 | Huwe 1 | 0.22 | 0.25 | 0.21 | 0.23 | 0.2 | 0.22 | 0.25 | 0.22 | 0.19 | 0.17 |
| chr9:31157695:31157847:-@chr9:31156190:31156225:- | NM_00 110245 | Aplp2 | 0.85 | 0.8 | 0.81 | 0.8 | 0.82 | 0.85 | 0.82 | 0.79 | 0.82 | 0.84 |
| @chr9:31153414:31153499:- | 5 | | | | | | | | | | | |
| chr3:146458743:146458840:+@chr3: 146459679:146459840:+@chr3:14646 3899:146465771:+ | NM_00 129367 2 | Ctbs | 0.9 | 0.84 | 0.81 | 0.85 | 0.85 | 0.9 | 0.79 | 0.79 | 0.69 | 0.88 |
| chr11:70609166:70609254:+@chr11: 70609408:70609431:+@chr11:706095 54:70609717:+ | NM_01 6713 | Mink1 | 0.94 | 0.92 | 0.87 | 0.88 | 0.93 | 0.92 | 0.77 | 0.81 | 0.87 | 0.86 |
| chr4:11287475:11287633:-@chr4:11285078:11285173:-@chr4:11281004:11281124:- | NM_00 108120 1 | Dpy19 14 | 0.84 | 0.81 | 0.78 | 0.82 | 0.74 | 0.88 | 0.82 | 0.89 | 0.85 | 0.81 |
| chr11:28434081:28434157:-@chr11:28396046:28396165:-@chr11:28385692:28388948:- | NM_00 116666 1 | Ccdc8 5a | 0.82 | 0.75 | 0.75 | 0.74 | 0.84 | 0.84 | 0.82 | 0.76 | 0.75 | 0.81 |
| chr4:9635901:9635969:-@chr4:9624360:9624401:-@chr4:9610843:9610938:- | NM_02 3066 | Asph | 0.12 | 0.09 | 0.15 | 0.15 | 0.1 | 0.1 | 0.18 | 0.07 | 0.2 | 0.2 |
| chr8:77155202:77155318:+@chr8:77 187520:77187664:+@chr8:77210644: 77210774:+ | NM_00 108390 6 | Nr3c2 | 0.85 | 0.82 | 0.76 | 0.72 | 0.89 | 0.87 | 0.82 | 0.83 | 0.81 | 0.9 |
| chr13:107022012:107022114:-@chr13:106996747:106997129:-@chr13:106993848:106993942:- | NM_00 114577 9 | Kif2a | 0.12 | 0.08 | 0.13 | 0.11 | 0.11 | 0.11 | 0.18 | 0.07 | 0.24 | 0.05 |
| chr7:109960074:109960422:-@chr7:109935482:109935553:-@chr7:109934693:109934802:- | NM_02 1494 | Dennd 5a | 0.51 | 0.44 | 0.56 | 0.49 | 0.53 | 0.55 | 0.71 | 0.61 | 0.51 | 0.54 |
| chr10:81314992:81315118:+@chr10: 81317326:81317403:+@chr10:813177 66:81319974:+ | NM_00 114668 7 | Pip5k1 c | 0.76 | 0.77 | 0.66 | 0.68 | 0.72 | 0.76 | 0.69 | 0.69 | 0.74 | 0.64 |
| chrX:74337911:74338054:+@chrX:74 338134:74338368:+@chrX:74338459: 74338601:+ | NM_00 8883 | Plxna3 | 0.81 | 0.83 | 0.83 | 0.87 | 0.89 | 0.89 | 0.86 | 0.86 | 0.83 | 0.76 |
| chrX:95166424:95166486:-@chrX:95165593:95165970:-@chrX:95130836:95131015:- | NM_00 129038 5 | Arhgef 9 | 0.9 | 0.87 | 0.89 | 0.94 | 0.91 | 0.87 | 0.77 | 0.92 | 0.95 | 0.88 |
| chrX:152178964:152179090:+@chrX: 152180997:152181092:+@chrX: 1522 02908:152203169:+ | NM_00 111466 4 | Iqsec2 | 0.17 | 0.18 | 0.15 | 0.1 | 0.16 | 0.29 | 0.09 | 0.09 | 0.19 | 0.19 |
| chr16:32898878:32898939:+@chr16: 32900633:32900707:+@chr16:329024 19:32902545:+ | NM_00 115934 9 | Fyttd1 | 0.85 | 0.84 | 0.74 | 0.88 | 0.85 | 0.95 | 0.78 | 0.92 | 0.83 | 0.85 |
| chr11:75349669:75349814:+@chr11: 75382250:75382394:+@chr11:753874 11:75387500:+ | NM_00 110261 1 | Smyd4 | 0.63 | 0.65 | 0.64 | 0.83 | 0.75 | 0.71 | 0.68 | 0.78 | 0.77 | 0.68 |
| chr4:129600983:129601134:+@chr4: | NM_02 | Tmem | 0.41 | 0.36 | 0.45 | 0.25 | 0.47 | 0.36 | 0.28 | 0.33 | 0.51 | 0.27 |
| 129601407:129601533:+@chr4:12960 2195:129602261:+ | 9748 | 234 | | | | | | | | | | |
| chr2:31950529:31950590:+@chr2:31 962215:31962281:+@chr2:31969630: 31969792:+ | NM_14 5144 | Aifll | 0.75 | 0.69 | 0.71 | 0.56 | 0.69 | 0.51 | 0.44 | 0.59 | 0.36 | 0.72 |
| chr5:81771584:81771680:+@chr5:81 792361:81792412:+@chr5:81794264: 81795347:+ | NM_19 8702 | Adgr13 | 0.37 | 0.21 | 0.21 | 0.41 | 0.29 | 0.26 | 0.32 | 0.26 | 0.34 | 0.35 |

TABLE 4, continued.

| | | | Acute | | | |
|---|---|---|---|---|---|---|
| #Event | gene | symb | 2567_mean | 2786_mean | 2898_mean | 2977_mean |
| chr2:38514581:38514658:+@chr2:38514733:38514864:+@chr2:38 550553:38550671:+ | NM_02160 6 | Nek6 | 0.2 | 0.14 | 0.18 | 0.21 |
| chr15:93410692:93410783:+@chr15:93420284:93420490:+@chr15 :93424014:93424075:+ | NM_00128 5864 | Pphln1 | 0.04 | 0.26 | 0.16 | 0.08 |
| chr3:81036416:81037554:+@chr3:81131018:81131076:+@chr3:81 141405:81141600:+ | NM_01997 1 | Pdgfc | 0.37 | 0.3 | 0.35 | 0.17 |
| chr2:32244234:32244389:+@chr2:32244786:32244950:+@chr2:32 245547:32245677:+ | NM_14514 5 | Pomt1 | 0.34 | 0.21 | 0.17 | 0.45 |
| chr19:40344356:40344439:-@chr19:40340045:40340146:-@chr19:40336986:40337035:- | NM_00103 4964 | Sorbs 1 | 0.79 | 0.53 | 0.51 | 0.45 |
| chr2:79657147:79658349:+@chr2:79658670:79658846:+@chr2:79 660233:79660310:+ | NM_08055 8 | Ssfa2 | 0.17 | 0.27 | 0.24 | 0.12 |
| chr19:4161068:4161215:-@chr19:4159566:4159705:-@chr19:4159403:4159460:- | NM_02148 5 | Rps6kb2 | 0.5 | 0.57 | 0.39 | 0.43 |
| chrX:74018130:74018366:-@chrX:74017901:74018017:-@chrX:74017130:74017511:- | NM_00117 7976 | Irak1 | 0.16 | 0.11 | 0.17 | 0.08 |
| chr11:31908825:31908875:+@chr11:31918801:31918824:+@chr11 :31920409:31920582:+ | NM_00129 0676 | Cpeb4 | 0.48 | 0.54 | 0.49 | 0.55 |
| chr6:29361294:29361487:+@chr6:29361560:29361753:+@chr6:29 366040:29366206:+ | NM_00128 5412 | Calu | 0.38 | 0.56 | 0.4 | 0.38 |
| chr5:5556065:5556156:-@chr5:5555238:5555406:-@chr5:5546353:5546405:- | NM_15311 6 | Gtpbp10 | 0.72 | 0.62 | 0.63 | 0.71 |
| chr8:127409556:127409707:+@chr8:127410729:127410773:+@chr 8:127415570:127415797:+ | NM_03362 0 | Pard3 | 0.48 | 0.58 | 0.35 | 0.44 |
| chr17:56686006:56686061:+@chr17:56692521:56692721:+@chr17 :56696602:56696638:+ | NM_02793 3 | Ranbp3 | 0.17 | 0.12 | 0.19 | 0.24 |
| chr7:27507947:27508103:+@chr7:27515251:27515447:+@chr7:27 516040:27520041:+ | NM_01941 2 | Prx | 0.41 | 0.37 | 0.57 | 0.48 |
| chr6:112644588:112644747:-@chr6:112628462:112628524:-@chr6:112619851:112620904:- | NM_00116 7730 | Rad18 | 0.47 | 0.46 | 0.51 | 0.5 |
| chr6:149310322:149310377:+@chr6:149324732:149324800:+@chr 6:149325405:149329784:+ | NM_02605 4 | 2810474 O19Rik | 0.13 | 0.23 | 0.22 | 0.29 |
| chr15:34117880:34118110:+@chr15:34121423:34121512:+@chr15 :34123668:34123766:+ | NM_02600 2 | Mtdh | 0.21 | 0.19 | 0.2 | 0.17 |
| chr2:124298575:124298655:+@chr2:124400243:124400365:+@chr 2:124489985:124490012:+ | NM_00129 0997 | Sema6d | 0.33 | 0.18 | 0.22 | 0.17 |
| chr16:59618795:59618850:-@chr16:59618666:59618678:-@chr16:59613321:59613844:- | NM_01966 5 | Arl6 | 0.15 | 0.13 | 0.12 | 0.16 |
| chr17:15499888:15500024:+@chr17:15501154:15501202:+@chr17 : 15502962:15503128:+ | NM_01368 4 | Tbp | 0.45 | 0.3 | 0.3 | 0.43 |
| chr16:3984991:3987176:-@chr16:3982979:3983242:-@chr16:3981585:3981687:- | NM_17747 2 | Slx4 | 0.13 | 0.17 | 0.09 | 0.05 |
| chr2:22957018:22957118:-@chr2:22950184:22950270:-@chr2:22946576:22946761:- | NM_00107 7190 | Abil | 0.37 | 0.42 | 0.38 | 0.36 |
| chr18:12862107:12862713:-@chr18:12841184:12841346:-@chr18:12819505:12819661:- | NM_00125 2492 | Osbplla | 0.77 | 0.69 | 0.64 | 0.78 |
| chr10:19904563:19904713:-@chr10:19894725:19894802:-@chr10:19894250:19894358:- | NM_00116 1825 | Pex7 | 0.8 | 0.62 | 0.78 | 0.74 |
| chr1:193362013:193362133:-@chr1:193361580:193361667:-@chr1:193359719:193359847:- | NM_14481 7 | Camklg | 0.08 | 0.06 | 0.11 | 0.07 |
| chr13:58160152:58160238:+@chr13:58162838:58162881:+@chr13 :58163433:58164693:+ | NM_19800 4 | Idnk | 0.11 | 0.07 | 0.11 | 0.11 |
| chr8:69744461:69744587:-@chr8:69744192:69744252:-@chr8:69742859:69743269:- | NM_00120 0023 | Zfp963 | 0.44 | 0.47 | 0.46 | 0.42 |
| chrX:50573224:50573351:-@chrX:50573015:50573141:-@chrX:50569277:50570291:- | NR_015505 | Firre | 0.66 | 0.59 | 0.63 | 0.77 |
| chr5:5557213:5557406:-@chr5:5555238:5555382:-@ chr5:5547927:5548000:- | NM_15311 6 | Gtpbp10 | 0.75 | 0.72 | 0.79 | 0.73 |
| chr16:87914154:87914404:-@chr16:87896809:87896895:-@chr16:87895897:87896351:- | NM_01034 8 | Grik1 | 0.34 | 0.38 | 0.34 | 0.36 |
| chr14:47704187:47704277:+@chr14:47704420:47704488:+@chr14 :47705763:47705800:+ | NM_00129 3636 | Ktn1 | 0.29 | 0.36 | 0.28 | 0.16 |
| chr10:14760289:14760710:+@chr10:14766677:14767025:+@chr10 : 14770155:14770556:+ | NM_00870 3 | Nmbr | 0.59 | 0.75 | 0.74 | 0.73 |
| chr11:4118625:4118729:-@chr11:4116681:4116756:-@chr11:4111894:4111937:- | NM_14452 0 | Sec1412 | 0.85 | 0.94 | 0.81 | 0.84 |
| chr19:4811172:4811634:-@chr19:4793926:4794009:-@chr19:4792398:4792820:- | NM_00129 0127 | Gm2199 2 | 0.5 | 0.35 | 0.46 | 0.69 |
| chr17:56011866:56011962:+@chr17:56012271:56012492:+@chr17 :56014941:56015109:+ | NM_02653 0 | Mpnd | 0.96 | 0.93 | 0.9 | 0.89 |
| chr4:91260761:91260986:-@chr4:91254141:91254179:-@chr4:91250767:91253570:- | NM_00117 7883 | Elavl2 | 0.9 | 0.92 | 0.91 | 0.86 |
| chrX:71376887:71376910:+@chrX:71379014:71379064:+@chrX:7 1383200:71383275:+ | NM_01698 5 | Mtmr1 | 0.4 | 0.41 | 0.37 | 0.58 |
| chr17:85615594:85615743:-@chr17:85614648:85614727:-@chr17:85609090:85611748:- | NR_015387 | Six3os1 | 0.44 | 0.57 | 0.47 | 0.48 |
| chr7:96694770:96695025:+@ chr7:96702642:96702740:+@ chr7:96 703926:96704161:+ | NM_01185 8 | Tenm4 | 0.42 | 0.65 | 0.56 | 0.44 |
| chr6:42350946:42351148:+@chr6:42351270:42351362:+@chr6:42 351475:42351969:+ | NM_00128 9618 | Zyx | 0.57 | 0.71 | 0.56 | 0.73 |
| chr8:3606267:3606406:+@chr8:3606504:3606820:+@chr8:360779 1:3607890:+ | NM_00116 3749 | Camsap3 | 0.89 | 0.93 | 0.83 | 0.87 |
| chr5:140713053:140713172:+@chr5:140714293:140714384:+@chr 5:140714567:140714685:+ | NM_00127 6287 | Brat1 | 0.76 | 0.8 | 0.62 | 0.9 |
| chr11:102224734:102224955:-@chr11:102219145:102219281:-@chr11:102218416:102218781:- | NM_00128 4249 | Hdac5 | 0.42 | 0.42 | 0.45 | 0.63 |
| chr15:99319131:99319254:-@chr15:99318918:99319017:-@chr15:99317223:99318372:- | NM_01171 1 | Fmnl3 | 0.9 | 0.91 | 0.86 | 0.56 |
| chr2:124298575:124298655:+@chr2:124400243:124400364:+@chr 2:124489985:124490012:+ | NM_00129 0997 | Sema6d | 0.27 | 0.17 | 0.17 | 0.17 |
| chr5:32785549:32785626:-@chr5:32774396:32774466:-@chr5:32764773:32764948:- | NM_17729 8 | Pisd | 0.81 | 0.76 | 0.77 | 0.69 |
| chrX:60295189:60295292:-@chrX:60293607:60293657:-@chrX:60292319:60292383:- | NM_00103 7863 | Atpllc | 0.83 | 0.89 | 0.79 | 0.82 |
| chr7:4010466:4010503:-@chr7:4010251:4010300:-@chr7:4007073:4009834:- | NM_00130 2675 | Lair1 | 0.65 | 0.78 | 0.82 | 0.83 |
| chr4:21746656:21746774:+@chr4:21747483:21747576:+@chr4:21 748137:21750546:+ | NM_00129 0420 | Ccnc | 0.58 | 0.58 | 0.67 | 0.64 |
| chr2:157560448:157560535:+@chr2:157561213:157561293:+@chr 2:157561542:157562519:+ | NM_01092 3 | Nnat | 0.48 | 0.75 | 0.61 | 0.61 |
| chr19:12577090:12577170:+@chr19:12583923:12584122:+@chr19 : 12586835:12589696:+ | NM_02664 0 | Famllla | 0.66 | 0.81 | 0.85 | 0.88 |
| chr17:94747617:94748417:-@chr17:94743932:94743994:-@chr17:94740386:94740755:- | NM_17691 7 | Mettl4 | 0.89 | 0.7 | 0.74 | 0.76 |
| chr12:101082451:101084025:+@chr12:101084284:101084499:+@c hr12:101088042:101088927:+ | NR_038049 | D130020 L05Rik | 0.54 | 0.6 | 0.66 | 0.67 |
| chr6:145211170:145211244:+@chr6:145214522:145214784:+@chr 6:145215184:145215271:+ | NM_00116 3628 | Etfrf1 | 0.77 | 0.6 | 0.74 | 0.74 |
| chr7:73514090:73514151:-@chr7:73507806:73507913:-@chr7:73504988:73505128:- | NM_00108 1345 | Chd2 | 0.49 | 0.53 | 0.52 | 0.29 |
| chr10: 128645721: 128645993:-@chr10:128643875:128643968:-@chr10:128642629:128642733:- | NM_01177 2 | Ikzf4 | 0.55 | 0.24 | 0.54 | 0.42 |
| chr8:85841002:85841103:+@chr8:85843021:85843112:+@chr8:85 875634:85875723:+ | NM_19944 6 | Phkb | 0.54 | 0.26 | 0.57 | 0.59 |
| chr18:59062381:59062562:+@chr18:59065835:59065885:+@chr18 :59072193:59072417:+ | NR_033765 | A730017 C20Rik | 0.62 | 0.43 | 0.53 | 0.64 |
| chr7:123179717:123179859:+@chr7:123180249:123180395:+@chr 7:123180540:123180677:+ | NM_14492 5 | Tnrc6a | 0.31 | 0.43 | 0.34 | 0.26 |
| chr11:95046921:95047046:-@chr11:95046449:95046590:-@chr11:95044482:95045763:- | NM_00130 6071 | Itga3 | 0.51 | 0.4 | 0.7 | 0.34 |
| chrX:135751945:135752017:+@chrX:135796142:135796336:+@ch rX: 135797553:135797629:+ | NM_02608 1 | Gprasp1 | 0.18 | 0.2 | 0.16 | 0.18 |
| chr4:118234959:118235103:-@chr4:118231486:118231791:-@chr4:118230695:118230888:- | NM_01121 3 | Ptprf | 0.25 | 0.28 | 0.44 | 0.51 |
| chr16:45408856:45409053:-@chr16:45400222:45400303:-@chr16:45396969:45397295:- | NM_01081 8 | Cd200 | 0.83 | 0.82 | 0.83 | 0.69 |
| chr2:71228600:71228690:+@chr2:71233649:71233708:+@chr2:71 235915:71236030:+ | NM_01006 4 | Dyncli2 | 0.78 | 0.75 | 0.74 | 0.64 |
| chrX: 151835224:151835292:+@chrX: 151836222:151836329:+@ch rX: 151840056:151840154:+ | NM_02152 3 | Huwel | 0.08 | 0.17 | 0.12 | 0.15 |
| chr9:31157695:31157847:-@chr9:31156190:31156225:-@chr9:31153414:31153499:- | NM_00110 2455 | Aplp2 | 0.76 | 0.71 | 0.67 | 0.8 |
| chr3:146458743:146458840:+@chr3:146459679:146459840:+@chr 3:146463899:146465771:+ | NM_00129 3672 | Ctbs | 0.92 | 0.86 | 0.94 | 0.91 |
| chr11:70609166:70609254:+@chr11:70609408:70609431:+@chr11 :70609554:70609717:+ | NM_01671 3 | Mink1 | 0.85 | 0.9 | 0.72 | 0.73 |
| chr4:11287475:11287633:-@chr4:11285078:11285173:-@chr4:11281004:11281124:- | NM_00108 1201 | Dpyl914 | 0.74 | 0.78 | 0.69 | 0.82 |
| chr11:28434081:28434157:-@chr11:28396046:28396165:-@chr11:28385692:28388948:- | NM_00116 6661 | Ccdc85a | 0.83 | 0.82 | 0.87 | 0.89 |
| chr4:9635901:9635969:-@chr4:9624360:9624401:-@chr4:9610843:9610938:- | NM_02306 6 | Asph | 0.11 | 0.04 | 0.05 | 0.09 |
| chr8:77155202:77155318:+@chr8:77187520:77187664:+@chr8:77 210644:77210774:+ | NM_00108 3906 | Nr3c2 | 0.7 | 0.83 | 0.69 | 0.84 |
| chr13:107022012:107022114:-@chr13:106996747:106997129:-@chr13:106993848:106993942:- | NM_00114 5779 | Kif2a | 0.17 | 0.22 | 0.19 | 0.14 |
| chr7:109960074:109960422:-@chr7:109935482:109935553:-@chr7:109934693:109934802:- | NM_02149 4 | Dennd5a | 0.56 | 0.59 | 0.69 | 0.57 |
| chr10:81314992:81315118:+@chr10:81317326:81317403:+@chr10 :81317766:81319974:+ | NM_00114 6687 | Pip5klc | 0.62 | 0.7 | 0.62 | 0.68 |
| chrX:74337911:74338054:+@chrX:74338134:74338368:+@chrX:7 4338459:74338601:+ | NM_00888 3 | Plxna3 | 0.92 | 0.9 | 0.9 | 0.83 |
| chrX:95166424:95166486:-@chrX:95165593:95165970:-@chrX:95130836:95131015:- | NM_00129 0385 | Arhgef9 | 0.71 | 0.93 | 0.85 | 0.9 |
| chrX:152178964:152179090:+@chrX:152180997:152181092:+@ch rX: 152202908:152203169:+ | NM_00111 4664 | Iqsec2 | 0.16 | 0.2 | 0.18 | 0.2 |
| chr16:32898878:32898939:+@chr16:32900633:32900707:+@chr16 :32902419:32902545:+ | NM_00115 9349 | Fyttd1 | 0.69 | 0.88 | 0.9 | 0.86 |
| chr11:75349669:75349814:+@chr11:75382250:75382394:+@chr11 :75387411:75387500:+ | NM_00110 2611 | Smyd4 | 0.76 | 0.74 | 0.74 | 0.69 |
| chr4:129600983:129601134:+@chr4:129601407:129601533:+@chr 4:129602195:129602261:+ | NM_02974 8 | Tmem23 4 | 0.34 | 0.36 | 0.31 | 0.34 |
| chr2:31950529:31950590:+@chr2:31962215:31962281:+@chr2:31 969630:31969792:+ | NM_14514 4 | Aifll | 0.6 | 0.7 | 0.71 | 0.65 |
| chr5:81771584:81771680:+@chr5:81792361:81792412:+@chr5:81 794264:81795347:+ | NM_19870 2 | Adgrl3 | 0.25 | 0.19 | 0.18 | 0.3 |

TABLE 4, continued.

| #Event | gene | symb | Acute Δpsi | Acute Stat | Acute Pval | c9 Δpsi | c9Stat | C9Pval |
|---|---|---|---|---|---|---|---|---|
| chr2:38514581:38514658:+@chr2:38514733:38514864: +@chr2:38550553:38550671:+ | NM_0 21606 | Nek6 | - 0.3841 66667 | - 2.1213 20344 | 0.0338 94854 | - 0.1596 66667 | - 2.2819 16488 | 0.0224 94271 |
| chr15:93410692:93410783:+@chr15:93420284:9342049 0:+@chr15:93424014:93424075:+ | NM_0 01285 864 | Pphln 1 | -0.288333333 | -2.121320344 | 0.033894854 | -0.249333333 | -2.366431913 | 0.017960478 |
| chr3:81036416:81037554:+@chr3:81131018:81131076: +@chr3:81141405:81141600:+ | NM_0 19971 | Pdgfc | -0.285833333 | -2.121320344 | 0.033894854 | -0.246333333 | -2.535462764 | 0.011229887 |
| chr2:32244234:32244389:+@chr2:32244786:32244950: +@chr2:32245547:32245677:+ | NM_1 45145 | Pomt 1 | -0.284166667 | -2.121320344 | 0.033894854 | -0.212666667 | -2.450947339 | 0.01424808 |
| chr19:40344356:40344439:-@chr19:40340045:40340146:- | NM_0 01034 | Sorbs 1 | -0.2733 | -2.1213 | 0.033894854 | -0.1043 | -2.5354 | 0.011229887 |
| @chr19:40336986:40337035:- | 964 | | 33333 | 20344 | | 33333 | 62764 | |
| chr2:79657147:79658349:+@chr2:79658670:79658846: +@chr2:79660233:79660310:+ | NM_0 80558 | Ssfa2 | -0.263333333 | -2.121320344 | 0.033894854 | -0.154333333 | -2.366431913 | 0.017960478 |
| chr19:4161068:4161215:-@chr19:4159566:4159705:-@chr19:4159403:4159460:- | NM_0 21485 | Rps6 kb2 | -0.260833333 | -2.121320344 | 0.033894854 | -0.156333333 | -2.535462764 | 0.011229887 |
| chrX:74018130:74018366:-@chrX:74017901:74018017:-@chrX:74017130:74017511:- | NM_0 01177 976 | Irak1 | -0.14 | 2.121320344 | 0.033894854 | -0.154 | 2.281916488 | 0.022494271 |
| chr11:31908825:31908875:+@chr11:31918801:3191882 4:+@chr11:31920409:31920582:+ | NM_0 01290 676 | Cpeb 4 | -0.245 | - 2.121320344 | 0.033894854 | -0.156 | - 2.197401062 | 0.027991815 |
| chr6:29361294:29361487:+@chr6:29361560:29361753: +@chr6:29366040:29366206:+ | NM_0 01285 412 | Calu | 0.243333333 | - 2.121320344 | 0.033894854 | - 0.207333333 | - 2.366431913 | 0.017960478 |
| chr5:5556065:5556156:-@chr5:5555238:5555406:-@chr5:5546353:5546405:- | NM_1 53116 | Gtpb p10 | 0.113333333 | 2.121320344 | 0.033894854 | 0.122333333 | 2.197401062 | 0.027991815 |
| chr8:127409556:127409707:+@chr8:127410729:127410 773:+@chr8:127415570:127415797:+ | NM_0 33620 | Pard 3 | 0.2375 | -2.121320344 | 0.033894854 | -0.185 | - 2.535462764 | 0.011229887 |
| chr17:56686006:56686061:+@chr17:56692521:5669272 1:+@chr17:56696602:56696638:+ | NM_0 27933 | Ranb p3 | - 0.226666667 | -2.121320344 | 0.033894854 | - 0.201666667 | -2.112885637 | 0.034610558 |
| chr7:27507947:27508103:+@chr7:27515251:27515447: +@chr7:27516040:27520041:+ | NM_0 19412 | Prx | -0.225833333 | -2.121320344 | 0.033894854 | -0.193333333 | -2.197401062 | 0.027991815 |
| chr6:112644588:112644747:-@chr6:112628462:112628524:-@chr6:112619851:112620904:- | NM_0 01167 730 | Rad1 8 | 0.201666667 | -2.121320344 | 0.033894854 | -0.201666667 | -2.112885637 | 0.034610558 |
| chr6:149310322:149310377:+@chr6:149324732:149324 800:+@chr6:149325405:149329784:+ | NM_0 26054 | 2810 474O 19Rik | -0.195833333 | -2.121320344 | 0.033894854 | -0.154333333 | -2.028370211 | 0.042522478 |
| chr15:34117880:34118110:+@chr15:34121423:3412151 2:+@chr15:34123668:34123766:+ | NM_0 26002 | Mtdh | -0.184166667 | -2.121320344 | 0.033894854 | -0.125666667 | -2.366431913 | 0.017960478 |
| chr2:124298575:124298655:+@chr2:124400243:124400 365:+@chr2:124489985:124490012:+ | NM_0 01290 997 | Sema 6d | 0.118333333 | 2.121320344 | 0.033894854 | 0.124333333 | 2.197401062 | 0.027991815 |
| chr16:59618795:59618850:-@chr16:59618666:59618678:-@chr16:59613321:59613844:- | NM_0 19665 | Arl6 | -0.183333333 | -2.121320344 | 0.033894854 | -0.170333333 | -2.197401062 | 0.027991815 |
| chr17:15499888:15500024:+@chr17:15501154:1550120 2:+@chr17:15502962:15503128:+ | NM_0 13684 | Tbp | -0.176666667 | -2.121320344 | 0.033894854 | -0.171666667 | -2.197401062 | 0.027991815 |
| chr16:3984991:3987176:-@chr16:3982979:3983242:-@chr16:3981585:3981687:- | NM_1 77472 | Slx4 | - 0.1533 33333 | - 2.1213 20344 | 0.0338 94854 | - 0.1373 33333 | - 2.4509 47339 | 0.0142 4808 |
| chr2:22957018:22957118:-@chr2:22950184:22950270:-@chr2:22946576:22946761:- | NM_0 01077 190 | Abil | 0.1591 66667 | 2.1213 20344 | 0.0338 94854 | 0.1686 66667 | 2.1974 01062 | 0.0279 91815 |
| chr18:12862107:12862713:-@chr18:12841184:12841346:-@chr18:12819505:12819661:- | NM_0 01252 492 | Osbpl 1a | - 0.1466 66667 | - 2.1213 20344 | 0.0338 94854 | - 0.1116 66667 | - 2.4509 47339 | 0.0142 4808 |
| chr10:19904563:19904713:-@chr10:19894725:19894802:-@chr10:19894250:19894358:- | NM_0 01161 825 | Pex7 | -0.145 | -2.121320344 | 0.033894854 | -0.103 | -2.112885637 | 0.034610558 |
| chr1:193362013:193362133:-@chr1:193361580:193361667:-@chr1:193359719:193359847:- | NM_1 44817 | Camk 1g | -0.143333333 | -2.121320344 | 0.033894854 | -0.141333333 | -2.281916488 | 0.022494271 |
| chr13:58160152:58160238:+@chr13:58162838:5816288 1:+@chr13:58163433:58164693:+ | NM_1 98004 | Idnk | -0.14 | -2.121320344 | 0.033894854 | -0.113 | -2.112885637 | 0.034610558 |
| chr8:69744461:69744587:-@chr8:69744192:69744252:-@chr8:69742859:69743269:- | NM_0 01200 023 | Zfp96 3 | 0.110833333 | 2.121320344 | 0.033894854 | 0.110333333 | 2.366431913 | 0.017960478 |
| chrX:50573224:50573351:-@chrX:50573015:50573141:-@chrX:50569277:50570291:- | NR_0 15505 | Firre | 0.145833333 | 2.121320344 | 0.033894854 | 0.133333333 | 2.366431913 | 0.017960478 |
| chr5:5557213:5557406:-@chr5:5555238:5555382:-@ chr5:5547927:5548000:- | NM_1 53116 | Gtpb p10 | 0.154166667 | 2.121320344 | 0.033894854 | 0.106666667 | 2.028370211 | 0.042522478 |
| chr16:87914154:87914404:-@chr16:87896809:87896895:-@chr16:87895897:87896351:- | NM_0 10348 | Grik1 | -0.181666667 | -2.121320344 | 0.033894854 | -0.203666667 | -2.197401062 | 0.027991815 |
| chr14:47704187:47704277:+@chr14:47704420:4770448 8:+@chr14:47705763:47705800:+ | NM_0 01293 636 | Ktn1 | 0.159166667 | 2.121320344 | 0.033894854 | 0.100666667 | 2.197401062 | 0.027991815 |
| chr10:14760289:14760710:+@chr10:14766677:1476702 5:+@chr10:14770155:14770556:+ | NM_0 08703 | Nmbr | -0.124166667 | -2.121320344 | 0.033894854 | -0.167666667 | -2.281916488 | 0.022494271 |
| chr11:4118625:4118729:-@chr11:4116681:4116756:-@chr11:4111894:4111937:- | NM_1 44520 | Sec14 12 | 0.166666667 | 2.121320344 | 0.033894854 | 0.115666667 | 2.197401062 | 0.027991815 |
| chr19:4811172:4811634:-@chr19:4793926:4794009:-@chr19:4792398:4792820:- | NM_0 01290 127 | Gm2 1992 | 0.176666667 | 2.121320344 | 0.033894854 | 0.135666667 | 2.028370211 | 0.042522478 |
| chr17:56011866:56011962:+@chr17:56012271:5601249 2:+@chr17:56014941:56015109:+ | NM_0 26530 | Mpnd | 0.18 | 2.121320344 | 0.033894854 | 0.145 | 2.197401062 | 0.027991815 |
| chr4:91260761:91260986:-@chr4:91254141:91254179:-@chr4:91250767:91253570:- | NM_0 01177 883 | Elav12 | 0.194166667 | 2.121320344 | 0.033894854 | 0.118666667 | 2.197401062 | 0.027991815 |
| chrX:71376887:71376910:+@chrX:71379014:71379064: +@chrX:71383200:71383275:+ | NM_0 16985 | Mtmr 1 | 0.213333333 | 2.121320344 | 0.033894854 | 0.188333333 | 2.197401062 | 0.027991815 |
| chr17:85615594:85615743:-@chr17:85614648:85614727:-@chr17:85609090:85611748:- | NR_0 15387 | Six3o s1 | - 0.173333333 | - 2.121320344 | 0.033894854 | - 0.247333333 | - 2.450947339 | 0.01424808 |
| chr7:96694770:96695025:+@chr7:96702642:96702740: +@chr7:96703926:96704161:+ | NM_0 11858 | Tenm 4 | 0.170833333 | 2.121320344 | 0.033894854 | 0.187333333 | 2.535462764 | 0.011229887 |
| chr6:42350946:42351148:+@chr6:42351270:42351362: +@chr6:42351475:42351969:+ | NM_001289618 | Zyx | 0.2225 | 2.121320344 | 0.033894854 | 0.204 | 2.535462764 | 0.011229887 |
| chr8:3606267:3606406:+@chr8:3606504:3606820:+@ch r8:3607791:3607890:+ | NM_001163749 | Camsap3 | 0.223333333 | 2.121320344 | 0.033894854 | 0.167333333 | 2.112885637 | 0.034610558 |
| chr5:140713053:140713172:+@chr5:140714293:140714 384:+@chr5:140714567:140714685:+ | NM_001276287 | Brat1 | 0.2333 33333 | 2.1213 20344 | 0.0338 94854 | 0.2313 33333 | 2.5354 62764 | 0.0112 29887 |
| chr11:102224734:102224955:-@chr11:102219145:102219281:-@chr11:102218416:102218781:- | NM_0 01284 249 | Hdac 5 | 0.2566 66667 | 2.1213 20344 | 0.0338 94854 | 0.1796 66667 | 2.1974 01062 | 0.0279 91815 |
| chr15:99319131:99319254:-@chr15:99318918:99319017:-@chr15:99317223:99318372:- | NM_0 11711 | Fmn13 | 0.264166667 | 2.121320344 | 0.033894854 | 0.140666667 | 2.028370211 | 0.042522478 |
| chr2:124298575:124298655:+@chr2:124400243:124400 364:+@chr2:124489985:124490012:+ | NM_0 01290 997 | Sema 6d | 0.111666667 | 2.121320344 | 0.033894854 | 0.181666667 | 2.281916488 | 0.022494271 |
| chr5:32785549:32785626:-@chr5:32774396:32774466:-@chr5:32764773:32764948:- | NM_1 77298 | Pisd | 0.115833333 | 2.121320344 | 0.033894854 | 0.169333333 | 2.028370211 | 0.042522478 |
| chrX:60295189:60295292:-@chrX:60293607:60293657:-@chrX:60292319:60292383:- | NM_001037863 | Atp11c | 0.2725 | 2.121320344 | 0.033894854 | 0.167 | 2.028370211 | 0.042522478 |
| chr7:4010466:4010503:-@chr7:4010251:4010300:-@chr7:4007073:4009834:- | NM_001302675 | Lair1 | 0.3 | 2.121320344 | 0.033894854 | 0.146 | 2.112885637 | 0.034610558 |
| chr4:21746656:21746774:+@chr4:21747483:21747576: +@chr4:21748137:21750546:+ | NM_001290420 | Ccnc | 0.304166667 | 2.121320344 | 0.033894854 | 0.211666667 | 2.366431913 | 0.017960478 |
| chr2:157560448:157560535:+@chr2:157561213:157561 293:+@chr2:157561542:157562519:+ | NM_010923 | Nnat | 0.305833333 | 2.121320344 | 0.033894854 | 0.173333333 | 2.366431913 | 0.017960478 |
| chr19:12577090:12577170:+@chr19:12583923:1258412 2:+@chr19:12586835:12589696:+ | NM_0 26640 | Fam111a | 0.31 | 2.121320344 | 0.033894854 | 0.173 | 2.028370211 | 0.042522478 |
| chr17:94747617:94748417:-@chr17:94743932:94743994:-@chr17:94740386:94740755:- | NM_1 76917 | Mett1 4 | 0.379166667 | 2.121320344 | 0.033894854 | 0.242666667 | 2.535462764 | 0.011229887 |
| chr12:101082451:101084025:+@chr12:101084284:1010 84499:+@chr12:101088042:101088927:+ | NR_0 38049 | D130 020L 05Rik | - 0.1725 | - 2.121320344 | 0.033894854 | -0.3 | - 2.535462764 | 0.011229887 |
| chr6:145211170:145211244:+@chr6:145214522:145214 784:+@chr6:145215184:145215271:+ | NM_0 01163 628 | Etfrf1 | 0.369166667 | 2.121320344 | 0.033894854 | 0.256666667 | 2.197401062 | 0.027991815 |
| chr7:73514090:73514151:-@chr7:73507806:73507913:-@chr7:73504988:73505128:- | NM_0 01081 345 | Chd2 | -0.225833333 | -2.121320344 | 0.033894854 | -0.145333333 | -2.197401062 | 0.027991815 |
| chr10:128645721:128645993:-@chr10:128643875:128643968:-@chr10:128642629:128642733:- | NM_0 11772 | Ikzf4 | -0.305833333 | -2.121320344 | 0.033894854 | -0.160333333 | -2.028370211 | 0.042522478 |
| chr8:85841002:85841103:+@chr8:85843021:85843112: +@chr8:85875634:85875723:+ | NM_1 99446 | Phkb | -0.15 | -2.121320344 | 0.033894854 | -0.156 | -2.281916488 | 0.022494271 |
| chr18:59062381:59062562:+@chr18:59065835:5906588 5:+@chr18:59072193:59072417:+ | NR_0 33765 | A730 017C 20Rik | -0.201666667 | -2.121320344 | 0.033894854 | -0.196666667 | -2.112885637 | 0.034610558 |
| chr7:123179717:123179859:+@chr7:123180249:123180 395:+@chr7:123180540:123180677:+ | NM_1 44925 | Tnrc6 a | -0.341666667 | -2.121320344 | 0.033894854 | -0.192666667 | -2.028370211 | 0.042522478 |
| chr11:95046921:95047046:-@chr11:95046449:95046590:-@chr11:95044482:95045763:- | NM_0 01306 071 | Itga3 | 0.264166667 | 2.121320344 | 0.033894854 | 0.221666667 | 2.197401062 | 0.027991815 |
| chrX:135751945:135752017:+@chrX: 135796142:13579 6336:+@chrX: 135797553:135797629:+ | NM_0 26081 | Gpras p1 | -0.243333333 | -2.121320344 | 0.033894854 | 0.079333333 | -2.028370211 | 0.042522478 |
| chr4:118234959:118235103:-@chr4:118231486:118231791:-@chr4:118230695:118230888:- | NM_0 11213 | Ptprf | 0.183333333 | 2.121320344 | 0.033894854 | 0.054333333 | 2.028370211 | 0.042522478 |
| chr16:45408856:45409053:-@chr16:45400222:45400303:-@chr16:45396969:45397295:- | NM_010818 | Cd200 | -0.150833333 | -2.121320344 | 0.033894854 | 0.041333333 | -2.281916488 | 0.022494271 |
| chr2:71228600:71228690:+@chr2:71233649:71233708: +@chr2:71235915:71236030:+ | NM_0 10064 | Dync 1i2 | -0.145833333 | -2.121320344 | 0.033894854 | 0.058333333 | -2.112885637 | 0.034610558 |
| chrX:151835224:151835292:+@chrX: 151836222:15183 6329:+@chrX: 151840056:151840154:+ | NM_0 21523 | Huwe 1 | -0.126666667 | -2.121320344 | 0.033894854 | 0.040666667 | -2.197401062 | 0.027991815 |
| chr9:31157695:31157847:-@chr9:31156190:31156225:-@chr9:31153414:31153499:- | NM_0 01102 455 | Aplp 2 | -0.125 | - 2.121320344 | 0.033894854 | -0.04 | - 2.112885637 | 0.034610558 |
| chr3:146458743:146458840:+@chr3:146459679:146459 840:+@chr3:146463899:146465771:+ | NM_0 01293 672 | Ctbs | 0.1375 | 2.121320344 | 0.033894854 | 0.06 | 2.028370211 | 0.042522478 |
| chr11:70609166:70609254:+@chr11:70609408:7060943 1:+@chr11:70609554:70609717:+ | NM_0 16713 | Mink 1 | -0.146666667 | -2.121320344 | 0.033894854 | -0.069666667 | -2.281916488 | 0.022494271 |
| chr4:11287475:11287633:-@chr4:11285078:11285173:-@chr4:11281004:11281124:- | NM_001081201 | Dpy1914 | -0.1325 | -2.121320344 | 0.033894854 | -0.066 | -2.197401062 | 0.027991815 |
| chr11:28434081:28434157:-@chr11:28396046:28396165:-@chr11:28385692:28388948:- | NM_0 01166 661 | Ccdc 85a | 0.1525 | 2.121320344 | 0.033894854 | 0.088 | 2.028370211 | 0.042522478 |
| chr4:9635901:9635969:-@chr4:9624360:9624401:-@chr4:9610843:9610938:- | NM_0 23066 | Asph | -0.124166667 | -2.121320344 | 0.033894854 | -0.060666667 | -2.028370211 | 0.042522478 |
| chr8:77155202:77155318:+@chr8:77187520:77187664: +@chr8:77210644:77210774:+ | NM_0 01083 906 | Nr3c 2 | -0.131666667 | -2.121320344 | 0.033894854 | -0.069666667 | -2.112885637 | 0.034610558 |
| chr13:107022012:107022114:-@chr13:106996747:106997129:-@chr13:106993848:106993942:- | NM_0 01145 779 | Kif2a | 0.12 | 2.121320344 | 0.033894854 | 0.06 | 2.028370211 | 0.042522478 |
| chr7:109960074:109960422:-@chr7:109935482:109935553:-@chr7:109934693:109934802:- | NM_0 21494 | Denn d5a | 0.139166667 | 2.121320344 | 0.033894854 | 0.081666667 | 2.028370211 | 0.042522478 |
| chr10:81314992:81315118:+@chr10:81317326:8131740 3:+@chr10:81317766:81319974:+ | NM_0 01146 687 | Pip5k 1c | -0.135 | -2.121320344 | 0.033894854 | -0.079 | -2.028370211 | 0.042522478 |
| chrX:74337911:74338054:+@chrX:74338134:74338368: +@chrX:74338459:74338601:+ | NM_0 08883 | Plxna 3 | 0.124166667 | 2.121320344 | 0.033894854 | 0.079666667 | 2.281916488 | 0.022494271 |
| chrX:95166424:95166486:-@chrX:95165593:95165970:-@chrX:95130836:95131015:- | NM_0 01290 385 | Arhg ef9 | - 0.1125 | - 2.121320344 | 0.033894854 | -0.07 | - 2.535462764 | 0.011229887 |
| chrX:152178964:152179090:+@chrX: 152180997:15218 1092:+@chrX:152202908:152203169:+ | NM_0 01114 664 | Iqsec 2 | 0.115 | 2.121320344 | 0.033894854 | 0.091 | 2.112885637 | 0.034610558 |
| chr16:32898878:32898939:+@chr16:32900633:3290070 7:+@chr16:32902419:32902545:+ | NM_0 01159 349 | Fyttd 1 | - 0.104166667 | - 2.121320344 | 0.033894854 | - 0.087666667 | - 2.112885637 | 0.034610558 |
| chr11:75349669:75349814:+@chr11:75382250:7538239 4:+@chr11:75387411:75387500:+ | NM_0 01102 611 | Smyd 4 | -0.084166667 | -2.121320344 | 0.033894854 | -0.104666667 | -2.197401062 | 0.027991815 |
| chr4:129600983:129601134:+@chr4:129601407:129601 533:+@chr4:129602195:129602261:+ | NM_0 29748 | Tme m234 | 0.0875 | 2.121320344 | 0.033894854 | 0.119 | 2.197401062 | 0.027991815 |
| chr2:31950529:31950590:+@chr2:31962215:31962281: +@chr2:31969630:31969792:+ | NM_1 45144 | Aifll | - 0.071666667 | -2.121320344 | 0.033894854 | -0.134666667 | -2.028370211 | 0.042522478 |
| chr5:81771584:81771680:+@chr5:81792361:81792412: +@chr5:81794264:81795347:+ | NM_1 98702 | Adgr1 3 | 0.1 | 2.121320344 | 0.033894854 | 0.172 | 2.535462764 | 0.011229887 |

### Example 9: Sequestration of RNA Binding Proteins by Expansion Transcripts Was Not Observed in C9orf72 ALS/FTD.

In myotonic dystrophy, a well-established RNA gain-of-function disease, CUG or CCUG expansion RNAs sequester MBNL proteins into intranuclear foci preventing their normal function in regulating posttranscriptional processing, including alternative splicing and polyadenylation. As expected, sequence analyses of abnormally spliced exons from DM1 skeletal muscle are most significantly enriched for MBNL YGCY binding motifs (FIG. 14).

Sense and antisense nuclear RNA foci and the abundant alternative splicing events found in both C9-ALS patients and in the C9-500 mice suggest that sense and antisense G₄C₂ and G₂C₄ expansion RNAs sequester one or more RNA binding proteins (hereinafter “RBPs”). In contrast to DM1, sequence analyses of abnormal splicing events in C9(+) pre-symptomatic mice and acute mice show the enrichment of a much larger number and more diverse set of tetramer motifs, including AT and multiple types of GC-rich repeat motifs (FIG. 5E). A diverse set of repeat motifs are also enriched in the C9-ALS patient splicing data (FIG. 5E). In contrast to the prominent involvement of a single category of RBP in DM1, the multiple types of RNA binding motifs found in the dysregulated genes in C9orf72 ALS/FTD are consistent with the published literature and indicate that C9orf72 RNA dysregulation may involve a larger group of RNA binding proteins, or may reflect concurrent processes that prevent unambiguous identification of a single RBP driver of disease (FIG. 5E).

Taken together, these data are consistent with a model in which acute C9-BAC mice show widespread neurodegeneration and neuroinflammation which is characterized by global changes in gene expression and alternative splicing. The changes found in the C9-BAC mice are similar to those found in C9-ALS patients at end-stage disease. Additionally, C9(+) pre-symptomatic mice show widespread changes in neuropathology and/or gene expression. Alternative splicing changes found in acute C9-BAC mice are also found early in disease (FIG. 6). Since the acute C9-BAC mice mimic C9-ALS patients in the aforementioned ways, alternative splicing changes identified in C9(+) pre-symptomatic mice may be useful as biomarkers for predicting early disease in humans.

In summary, it is shown that alternative splicing changes are found in C9-BAC mice and increase with disease severity and hence may provide useful biomarkers and tools to understand disease progression. In contrast to DM1, motif enrichment analyses shows that the alternative splicing changes found in the C9-BAC mice are unlikely to be caused by sequestration of a single category of RBP.

### Discussion

More than a dozen independent mouse models have been developed to understand the complex molecular mechanisms of C9orf72 ALS/FTD. Among these, a BAC transgenic mouse model of C9orf72 ALS/FTD exists that is unique because it develops the behavioral, neuropathological, and molecular features of disease. To further understand disease in this model, whole genome sequencing was performed, and it was shown that four independent lines of these BAC mice have unique integration sites. RNA sequencing identified alternative splicing changes that affect RNA processing and degradation pathways as an early molecular signature of disease, which worsens with disease progression. Similar to C9orf72 ALS/FTD patients, gene expression changes in neuroinflammatory and neurodegenerative pathways predominate in severely affected end-stage animals. Finally, isogenic sub-lines generated from the single copy C9-500 line with 800 repeats (C9-800) show increased RNA foci and RAN protein aggregates as well as earlier ages of onset and increased disease penetrance. These data demonstrate that phenotypes in the C9-BAC mouse model are caused by the repeat expansion, occur independent of transgene integration sites, and that longer repeat lengths decrease age of onset and increase disease penetrance.

Patients with C9orf72 expansion mutations show remarkable heterogeneity in clinical presentation and age of onset, but the role of repeat length as modifier has been difficult to determine for a number of reasons. First, there is substantial somatic repeat instability and repeat lengths in blood are likely to be substantially shorter than those found in affected brain tissue. Southern blotting is the most common way to measure repeat length but somatic instability and the variation of repeat lengths within a single individual make comparisons of repeat length and age of onset in patients inaccurate. Additionally, GC-rich expansion mutations are difficult to amplify by PCR and to sequence making small-pool PCR strategies to accurately measure the distributions of repeat lengths challenging. The demonstration herein that repeat length modifies both age of onset and disease penetrance in an isogenic allelic series of FVB C9-BAC mice demonstrates that repeat length is an important modifier in C9 ALS/FTD. These results, combined with the somatic instability seen in patients, suggest that targeting DNA repair pathways may be a viable approach for mitigating disease in C9orf72 ALS/FTD. Additionally, the phenotypes seen here in animals with 50 repeats combined with previously published phenotypes in animals with 4 copies of the transgene containing 36-29 repeats suggest that additional human studies are needed to understand the relative risks of shorter repeat expansion mutations and the potential role of somatic instability. This may be particularly relevant for families with sporadic cases of C9orf72 ALS/FTD.

Ectopic cytoplasmic localization of mutant TDP43, FUS and MATR3 or overexpression of wildtype TDP43, FUS and MATR3 protein has been shown to be associated with ALS phenotypes in patients and model systems respectively. C9orf72, a DENN (differentially expressed in normal and neoplastic cells) domain containing protein, has been shown to play a role in autophagy and immune-regulatory functions. Recently, it has been shown that a dose dependent increase in motor deficits was observed when the single copy C9-500 mice disclosed herein are crossed with C9orf72 heterozygous and homozygous mice, suggesting that both loss of function and gain of function effects may contribute to C9orf72 ALS/FTD. Additionally, it has been proposed that overexpression of C9orf72 protein may lead to toxicity in mouse models of disease. The data disclosed herein, showing that there are no significant differences between C9-500 and NT mice combined with allelic series data, indicate that repeat length, and not C9orf72 overexpression, is the primary driver of the ALS/FTD phenotypes found in the mice disclosed herein.

Substantial data suggest RNA gain-of-function effects contribute to C9orf72 ALS/FTD and a large number of RNA binding proteins have been proposed to be sequestered by the repeats. Circular dichroism studies performed on short stretches of sense repeats G₄C₂ show that the repeat can adopt G quadruplex, R loop, or hairpin conformations, and the antisense repeats likely adopt a hairpin conformation. The variability and complexity in secondary structures formed by the repeats may make it more likely that multiple RNA binding proteins are sequestered by these repeat motifs, a possibility consistent with the present data showing that genes with abnormal splicing events have a variety of RNA recognition motifs. Since several of these predicted RNA binding proteins also contain low complexity domains, it is possible that multiple RNA binding proteins interact with repeat RNAs and form dynamic liquid-droplet like structures that may be complicated to resolve.

Transcriptomic data from the acute C9-BAC mice disclosed herein are consistent with gene expression changes caused by ongoing apoptotic processes involved in cell death and inflammation found at end-stage disease. In contrast, transcriptomic profiles of pre-symptomatic mice showed minimal changes in gene expression, but abundant changes in alternative splicing. Robust alternative splicing changes are also detected in neurons differentiated from C9orf72 patient iPSCs (iPSN), but show few gene expression differences compared to C9orf72 patient autopsy tissue. In the mice disclosed here, 240 genes are mis-spliced in C9(+) pre-symptomatic animals, and the psi values of 83 of these gene increase further in end-stage acute animals. These genes include Elavl2, which was shown to be pathogenic in a yeast functional screen. Elavl2 has an RNA recognition motif (RRM) and has been predicted to play a role in ALS because of its similarity to TDP-43 and FUS. Pard3, regulates neuronal polarity has also been shown to be mis-spliced in ALS patient autopsy tissue. These data, combined with the 83 other alternative spliced genes whose psi values increase with disease progression, highlight these genes as a novel and early molecular signature of C9orf72 ALS/FTD.

The role of RAN proteins in disrupting alternative splicing has also been proposed. In cell culture experiments, ~5,000 mis-spliced events were observed when astrocytes were treated with PR. Additionally, it has been shown that in cell culture systems, GR and PR associate with low complexity domains of RNA binding proteins and can cause mis-splicing in a U2snRNP-dependent manner. These low complexity domain proteins are typically involved in the formation of membrane-less organelles such as stress granules and neuronal speckles. Since splicing factors normally localize to the neuronal speckles, it is possible that RAN proteins interacting with RNA binding proteins containing low complexity domains may disrupt splicing globally, thereby causing an increase in overall alternatively spliced events rather than a specific set of events mediated by the sequestration of a single RNA binding protein as is seen in myotonic dystrophy. These data, combined with the present data showing that phenotypic C9-500 mice have more RAN protein aggregates compared to asymptomatic C9-500, suggest that RAN proteins could contribute to the early RNA splicing abnormalities found in the mice used here. Because longer repeat lengths result in the expression of both longer expansion RNAs and increased RAN protein aggregates, additional work will be needed to clarify the relative roles of RNA gain of function effects and RAN proteins in disease. It has recently been shown that antibodies that decrease sense RAN proteins in C9-BAC mice rescue behavior and survival in these mice, without changing the levels of their corresponding expansion RNAs, highlighting the therapeutic potential of targeting RAN proteins.

Since the discovery of the C9orf72 expansion mutation, research has focused on understanding disease mechanisms and identifying therapeutic targets. Transgenic mouse models have contributed to these efforts, but ectopic overexpression and transgene integration can affect the phenotypes that these mice develop and the suitability of these animals to test potential therapies. This issue has been shown to occur in the widely used R6/2 mouse model of Huntington disease. The C9-500 BAC transgenic mice disclosed herein are a valuable tool both for understanding the disease and to test novel therapeutic strategies. First, the phenotypes in the C9-500 mice, which contain the full-length C9orf72 gene and substantial flanking sequences, are not affected by integration site. Second, the C9-500 line carries one copy of the transgene expressed at levels comparable to the endogenous C9orf72 ortholog in mice. Third, a group of alternative splicing changes were identified that worsen with disease progression and may be useful biomarkers of disease. Finally, it is demonstrated here that repeat length increases disease penetrance and that the phenotypes in the mice are caused by a gain of function of the repeat expansion. Taken together, these data provide mechanistic insight into C9orf72 ALS/FTD and demonstrate that the C9-500 mice are a robust tool to test therapeutic strategies for disease intervention.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method for identifying a subject as having pre-symptomatic amyotrophic lateral sclerosis (ALS), the method comprising:
(i) detecting levels of two or more isoforms of one or more genes selected from: Nek6, Pphlnl, Pdgfc, Pomt1, Sorbs1, Ssfa2, Rps6kb2, Irak1, Cpeb4, Calu, GtpbplO, Pard3, Ranbp3, Prx, Rad18, 2810474O19Rik, Mtdh, Sema6d, Arl6, Tbp, Slx4, Abi1, Osbpl1a, Pex7, Camklg, Idnk, Zfp963, Firre, Grik1, Ktnl, Nmbr, Sec14l2, Gm21992, Mpnd, Elavl2, Mtmrl, Six3os1, Tenm4, Zyx, Camsap3, Brat1, Hdac5, Fmnl3, Pisd, Atp11c, Lair1, Ccnc, Nnat, Fam111a, Mettl4, D130020L05Rik, Etfrfl, Chd2, Ikzf4, Phkb, A730017C20Rik, Tnrc6a, Itga3, Gpraspl, Ptprf, Cd200, Dync1i2, Huwel, Aplp2, Ctbs, Minkl, Dpy1914, Ccdc85a, Asph, Nr3c2, Kif2a, Dennd5a, Pip5k1c, Plxna3, Arhgef9, Iqsec2, Fyttdl, Smyd4, Tmem234, Aif1l, Adgrl3, 0610037L13Rik, 1600014C10Rik, 1700001L05Rik, 3010001F23Rik, 6430573F11Rik, 9030617O03Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfdl, Cacnalc, Cadml, Cadps, Calcrl, Camkk2, Ccdc43, Ccdc66, Cd180, Cdk14, Celf2, Cep350, Cep89, Cfhr2, Chd5, Chl1, Ckmt1, Claspl, Clcn3, Clec16a, Clip2, Clk4, Clta, Cltb, Cmc4, Cnnm2, Cnot10, Cntn4, Coblll, Copg2, Coro6, Cp, Cpeb2, Cpne5, Crtc2, Csppl, Csrnp3, Ctnnd1, Cwc22, Dab2, Dcaf17, Dclkl, Dgkb, Dhrs3, Disc1, Dkc1, Dlgl, Dlg3, Dlgap4, Dnm2, Donson, Dst, Dtnb, Eefld, Efcab6, Eif2ak4, Eif4g1, Eml4, Entpd5, Eprs, Erbb4, Ercl, Ets1, Ewsr1, Exosc10, Fam107b, Fam13a, Fam149b, Fam151b, Fam227a, Fam3a, Fancc, Fbln2, Fbrsll, Fbxo34, Fcgr2b, Fgfrlop2, Flna, Fnl, Fxr1, Fyn, G3bp2, Galns, Glt8dl, Gm20319, Gm28042, Gng12, Golgbl, Gphn, Gpm6b, Gprin1, Gramd3, Gsn, H13, H2-Q7, H2-T22, Hist1h2bq, Hist1h2br, Hnrnpa2b1, Hrasls, Hrhl, Hsd3b3, Hsf4, Ifi27, Il10rb, Il15ra, Inf2, Inpp4a, Inppll, Ints10, Invs, Itgam, Itgb5, Itpr2, Itsnl, Itsn2, Kansll, Kantr, Kars, Kcnk2, Kcnt1, Klc4, Las1l, Lifr, Limk2, Lin54, Lin7a, Lpin1, Lrif1, Lrrc14, Lrrfipl, Map2k3, Map4, Map4k4, Map7d1, Mapkbpl, Mark4, Mast2, Mast4, Matk, Mbnl2, Med15, Med17, Med27, Megf11, Metapld, Mid1, Mknk1, Morf4l2, Morn1, Mpv17l2, Mthfsl, Myole, Myo6, Myrf, Myt1, N6amt1, Naa35, Nav3, Nckap5, Ncoal, Ncorl, Ndorl, Necap2, Nemp2, Nfl, Nfasc, Nfia, Nful, Nin, Nit1, Nkain4, Nkirasl, Nktr, Nme5, Nprl3, Nptn, Nrbpl, Nrcam, Nrf1, Nron, Nrxn1, Nrxn2, Nrxn3, Nsd2, Nsun4, Nup54, Osbpl8, Oscpl, Oxnad1, Patz1, Pbx3, Pcdh15, Pdzd9, Pecam1, Pex2, Phc3, Phldb1, Picalm, Pign, Pkp4, Plcd4, Plchl, Plcxd3, Plec, Pml, Pnkd, Pnpla6, Polr3gl, Porcn, Ppfial, Ppp1r16a, Ppp4r1l-ps, Prkabl, Prpsap2, Prr13, Prr14, Prrc2c, Prss53, Psap, Ptbpl, Ptbp3, Ptpre, Ptprk, Ptprt, R3hdm2, Rablla, Rabep2, Ralgapal, Raly, Rapgef1, Rassf2, Rbm10, Rbm12, Rbm39, Rbms3, Rcbtb2, Retreg3, Rffl, Rhobtbl, Rhoj, Ripor2, Rpain, Rpgr, Rps24, Rps6kal, Rtell, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1, Snap23, Snrpal, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd52l2, Trp53i13, Trpc1, Ttc17, Ttc21b, Ttll5, Tysnd1, Ube2cbp, Ubn2, Ubxn7, Uhrflbpl, Unc13b, Uqccl, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrfl, Zmat1, Zprl, Zranb3, Flnc, Msn, Serpina3n, Cd44, Stat3, Igfbp3, Cd63, Ucp2, Sppl, Plek, Col5a3, S1pr3, Anxa2, Mt1, Sbno2, Csf1, Ch25h, Mt2, Serpinel, Vim, Col16a1, Neat1, Cd109, Dysf, C3arl, Pros1, Tnfrsfla, Tgm1, Socs3, Rplp0, Osmr, Cebpd, Tspan7, Srxn1, Capg, Ahnak, Fgf13, S100a10, Arpc1b, Slc6a1, Lgals3, Bcl3, Plin2, Csf2rb2, Clic1, Gfap, Aspg, Empl, and/or Msr1 in a biological sample obtained from a subject;
(ii) calculating psi (Ψ) score for each of the one or more genes based on the detecting of (i);
(iii) comparing each Ψ score of (ii) to a Ψ score of the same gene from a control sample to produce a delta psi (ΔΨ) score; and,
(iv) identifying the subject as having pre-symptomatic ALS if the absolute value of the ΔΨ score is not zero (0).

2. The method of claim 1, wherein the biological sample is a blood sample, serum sample, or a tissue sample, optionally wherein the tissue sample is a CNS tissue sample or a cerebrospinal fluid (CSF) sample.

3. The method of claim 1 or 2, wherein the detecting comprises performing RNA sequencing (RNAseq) on the biological sample.

4. The method of any one of claims 1 to 3, wherein the calculating comprises performing a mixture of isoforms (MISO) analysis.

5. The method of any one of claims 1 to 4, wherein the control sample is a biological sample obtained from a healthy subject.

6. The method of any one of claims 1 to 4, wherein the control sample is a biological sample obtained from the same subject at an earlier point in time.

7. The method of any one of claims 1 to 6, wherein the subject is a mammal, optionally wherein the mammal is a human or a mouse.

8. The method of any one of claims 1 to 7, wherein the subject is characterized as having a C9orf72 expansion repeat greater than 30 repeats, or wherein the subject expresses one or more RAN proteins from a C9orf72 expansion repeat.

9. The method of any one of claims 1 to 8, wherein if the gene is Nek6, Pphlnl, Pdgfc, Pomt1, Sprbsl, Ssfa2, Rps6kb2, Cpeb4, Calu, Pard3, Ranpb3, Prx, 2810474O19Rik, Mtdh, Arl6, Tbp. Slx4, Osbplla, Pex7, Camklg, or Idnk, the delta psi (ΔΨ) score is a negative value.

10. The method of any one of claims 1 to 8, wherein if the gene is Zfp963, Firre, Gtpbp10, Ktnl, Sec1412, Gm21992, Mpnd, Elavl2, Mtmr1, Zyx, Camsap3, Brat1, Hdac5, Fmnl3, Atp11c, Liar1, Ccnc, Nnat, Fam111a, or Mettl4, the delta psi (ΔΨ) score is a positive value.

11. A method for monitoring disease progression in a subject, the method comprising:
(i) detecting in a first biological sample obtained from a subject (e.g., a subject that is genopositive for a C9orf72 expansion repeat) a first psi (Ψ) score of one or more genes selected from: Nek6, Pphlnl, Pdgfc, Pomt1, Sorbs1, Ssfa2, Rps6kb2, Irak1, Cpeb4, Calu, Gtpbp10, Pard3, Ranbp3, Prx, Rad18, 2810474O19Rik, Mtdh, Sema6d, Arl6, Tbp, Slx4, Abi1, Osbpl1a, Pex7, Camklg, Idnk, Zfp963, Firre, Grik1, Ktnl, Nmbr, Sec14l2, Gm21992, Mpnd, Elavl2, Mtmrl, Six3osl, Tenm4, Zyx, Camsap3, Brat1, Hdac5, Fmnl3, Pisd, Atp11c, Lair1, Ccnc, Nnat, Fam111a, Mettl4, D130020L05Rik, Etfrfl, Chd2, Ikzf4, Phkb, A730017C20Rik, Tnrc6a, Itga3, Gpraspl, Ptprf, Cd200, Dync1i2, Huwel, Aplp2, Ctbs, Minkl, Dpy1914, Ccdc85a, Asph, Nr3c2, Kif2a, Dennd5a, Pip5k1c, Plxna3, Arhgef9, Iqsec2, Fyttdl, Smyd4, Tmem234, Aif1l, Adgrl3, 0610037L13Rik, 1600014C10Rik, 1700001L05Rik, 3010001F23Rik, 6430573F11Rik, 9030617O03Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfdl, Cacnalc, Cadml, Cadps, Calcrl, Camkk2, Ccdc43, Ccdc66, Cd180, Cdk14, Celf2, Cep350, Cep89, Cfhr2, Chd5, Chl1, Ckmt1, Claspl, Clcn3, Clec16a, Clip2, Clk4, Clta, Cltb, Cmc4, Cnnm2, Cnot10, Cntn4, Coblll, Copg2, Coro6, Cp, Cpeb2, Cpne5, Crtc2, Csppl, Csrnp3, Ctnnd1, Cwc22, Dab2, Dcaf17, Dclkl, Dgkb, Dhrs3, Disc1, Dkc1, Dlgl, Dlg3, Dlgap4, Dnm2, Donson, Dst, Dtnb, Eefld, Efcab6, Eif2ak4, Eif4gl, Eml4, Entpd5, Eprs, Erbb4, Ercl, Ets1, Ewsr1, Exosc10, Fam107b, Fam13a, Fam149b, Fam151b, Fam227a, Fam3a, Fancc, Fbln2, Fbrsll, Fbxo34, Fcgr2b, Fgfrlop2, Flna, Fnl, Fxr1, Fyn, G3bp2, Galns, Glt8dl, Gm20319, Gm28042, Gng12, Golgbl, Gphn, Gpm6b, Gprin1, Gramd3, Gsn, H13, H2-Q7, H2-T22, Hist1h2bq, Hist1h2br, Hnrnpa2b1, Hrasls, Hrhl, Hsd3b3, Hsf4, Ifi27, Il10rb, Il15ra, Inf2, Inpp4a, Inppll, Ints10, Invs, Itgam, Itgb5, Itpr2, Itsnl, Itsn2, Kansll, Kantr, Kars, Kcnk2, Kcntl, Klc4, Las1l, Lifr, Limk2, Lin54, Lin7a, Lpin1, Lrifl, Lrrc14, Lrrfipl, Map2k3, Map4, Map4k4, Map7d1, Mapkbpl, Mark4, Mast2, Mast4, Matk, Mbnl2, Med15, Med17, Med27, Megf11, Metapld, Mid1, Mknk1, Morf4l2, Morn1, Mpv17l2, Mthfsl, Myo1e, Myo6, Myrf, Myt1, N6amt1, Naa35, Nav3, Nckap5, Ncoal, Ncorl, Ndorl, Necap2, Nemp2, Nfl, Nfasc, Nfia, Nful, Nin, Nit1, Nkain4, Nkiras1, Nktr, Nme5, Nprl3, Nptn, Nrbpl, Nrcam, Nrf1, Nron, Nrxn1, Nrxn2, Nrxn3, Nsd2, Nsun4, Nup54, Osbpl8, Oscpl, Oxnad1, Patz1, Pbx3, Pcdh15, Pdzd9, Pecam1, Pex2, Phc3, Phldbl, Picalm, Pign, Pkp4, Plcd4, Plchl, Plcxd3, Plec, Pml, Pnkd, Pnpla6, Polr3gl, Porcn, Ppfial, Ppp1r16a, Ppp4r1l-ps, Prkabl, Prpsap2, Prr13, Prr14, Prrc2c, Prss53, Psap, Ptbpl, Ptbp3, Ptpre, Ptprk, Ptprt, R3hdm2, Rablla, Rabep2, Ralgapal, Raly, Rapgef1, Rassf2, Rbm10, Rbm12, Rbm39, Rbms3, Rcbtb2, Retreg3, Rffl, Rhobtbl, Rhoj, Ripor2, Rpain, Rpgr, Rps24, Rps6kal, Rtell, Rtn4, Rubcn, Runx1t1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1, Snap23, Snrpal, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Ijap1, Tle3, Tlr4, Tlr7, Tmem134, Tmem209, Tmem25, Tmem44, Tpd52, Tpd52l2, Trp53i13, Trpc1, Ttc17, Ttc21b, Ttll5, Tysndl, Ube2cbp, Ubn2, Ubxn7, Uhrflbpl, Unc13b, Uqccl, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrfl, Zmat1, Zprl, Zranb3, Flnc, Msn, Serpina3n, Cd44, Stat3, Igfbp3, Cd63, Ucp2, Sppl, Plek, Col5a3, Slpr3, Anxa2, Mt1, Sbno2, Csf1, Ch25h, Mt2, Serpinel, Vim, Col16a1, Neat1, Cd109, Dysf, C3arl, Pros1, Tnfrsfla, Tgm1, Socs3, Rplp0, Osmr, Cebpd, Tspan7, Srxn1, Capg, Ahnak, Fgf13, S100a10, Arpc1b, Slc6al, Lgals3, Bcl3, Plin2, Csf2rb2, Clic1, Gfap, Aspg, Empl, and/or Msr1;
(ii) detecting in a second biological sample obtained from the subject a second psi (Ψ) score of one or more genes selected from: Nek6, Pphlnl, Pdgfc, Pomt1, Sorbs1, Ssfa2, Rps6kb2, Irak1, Cpeb4, Calu, Gtpbp10, Pard3, Ranbp3, Prx, Rad18, 2810474O19Rik, Mtdh, Sema6d, Arl6, Tbp, Slx4, Abi1, Osbpl1a, Pex7, Camklg, Idnk, Zfp963, Firre, Grik1, Ktn1, Nmbr, Sec14l2, Gm21992, Mpnd, Elavl2, Mtmrl, Six3osl, Tenm4, Zyx, Camsap3, Brat1, Hdac5, Fmnl3, Pisd, Atp11c, Lair1, Ccnc, Nnat, Fam111a, Mettl4, D130020L05Rik, Etfrfl, Chd2, Ikzf4, Phkb, A730017C20Rik, Tnrc6a, Itga3, Gpraspl, Ptprf, Cd200, Dync1i2, Huwel, Aplp2, Ctbs, Minkl, Dpy1914, Ccdc85a, Asph, Nr3c2, Kif2a, Dennd5a, Pip5k1c, Plxna3, Arhgef9, Iqsec2, Fyttdl, Smyd4, Tmem234, Aif1l, Adgrl3, 0610037L13Rik, 1600014C10Rik, 1700001L05Rik, 3010001F23Rik, 6430573F11Rik, 9030617O03Rik, 9430015G10Rik, 9530077C05Rik, A430005L14Rik, Aasdhppt, Abcb1b, Abhd5, Ablim2, Acin1, Acsl1, Adam22, Adam23, Aebp1, Afdn, Ago3, Ak4, Akap2, Alg11, Ankrd16, Ankrd24, Ankrd33b, Anks6, Anxa7, Ap4b1, Ap4e1, Apbb2, Aqp11, Arap1, Arfgef2, Arhgef1, Arhgef26, Arhgef7, Arid3b, Arpp21, Asb7, Atp11a, Atp13a5, Atrx, Atxn2, Bag6, Bbof1, Bcas3, Bend5, Bnc2, Brd2, Brd4, Btbd17, Cacfd1, Cacna1c, Cadm1, Cadps, Calcrl, Camkk2, Ccdc43, Ccdc66, Cd180, Cdk14, Celf2, Cep350, Cep89, Cfhr2, Chd5, Chl1, Ckmt1, Clasp1, Clcn3, Clec16a, Clip2, Clk4, Clta, Cltb, Cmc4, Cnnm2, Cnot10, Cntn4, Cobll1, Copg2, Coro6, Cp, Cpeb2, Cpne5, Crtc2, Csppl, Csrnp3, Ctnnd1, Cwc22, Dab2, Dcaf17, Dclk1, Dgkb, Dhrs3, Disc1, Dkc1, Dlgl, Dlg3, Dlgap4, Dnm2, Donson, Dst, Dtnb, Eefld, Efcab6, Eif2ak4, Eif4g1, Eml4, Entpd5, Eprs, Erbb4, Erc1, Ets1, Ewsr1, Exosc10, Fam107b, Fam13a, Fam149b, Fam151b, Fam227a, Fam3a, Fancc, Fbln2, Fbrsl1, Fbxo34, Fcgr2b, Fgfrlop2, Flna, Fn1, Fxr1, Fyn, G3bp2, Galns, Glt8d1, Gm20319, Gm28042, Gng12, Golgb1, Gphn, Gpm6b, Gprin1, Gramd3, Gsn, H13, H2-Q7, H2-T22, Hist1h2bq, Hist1h2br, Hnrnpa2b1, Hrasls, Hrh1, Hsd3b3, Hsf4, Ifi27, Il10rb, Il15ra, Inf2, Inpp4a, Inppl1, Ints10, Invs, Itgam, Itgb5, Itpr2, Itsn1, Itsn2, Kansl1, Kantr, Kars, Kcnk2, Kcnt1, Klc4, Lasl1, Lifr, Limk2, Lin54, Lin7a, Lpin1, Lrif1, Lrrc14, Lrrfip1, Map2k3, Map4, Map4k4, Map7d1, Mapkbp1, Mark4, Mast2, Mast4, Matk, Mbnl2, Med15, Med17, Med27, Megf11, Metap1d, Mid1, Mknk1, Morf4l2, Morn1, Mpv17l2, Mthfsl, Myo1e, Myo6, Myrf, Myt1, N6amt1, Naa35, Nav3, Nckap5, Ncoa1, Ncor1, Ndor1, Necap2, Nemp2, Nf1, Nfasc, Nfia, Nfu1, Nin, Nit1, Nkain4, Nkiras1, Nktr, Nme5, Nprl3, Nptn, Nrbp1, Nrcam, Nrf1, Nron, Nrxn1, Nrxn2, Nrxn3, Nsd2, Nsun4, Nup54, Osbpl8, Oscp1, Oxnad1, Patz1, Pbx3, Pcdh15, Pdzd9, Pecam1, Pex2, Phc3, Phldb1, Picalm, Pign, Pkp4, Plcd4, Plch1, Plcxd3, Plec, Pml, Pnkd, Pnpla6, Polr3gl, Porcn, Ppfia1, Ppp1r16a, Ppp4r1l-ps, Prkab1, Prpsap2, Prr13, Prr14, Prrc2c, Prss53, Psap, Ptbp1, Ptbp3, Ptpre, Ptprk, Ptprt, R3hdm2, Rab11a, Rabep2, Ralgapa1, Raly, Rapgef1, Rassf2, Rbm10, Rbm12, Rbm39, Rbms3, Rcbtb2, Retreg3, Rffl, Rhobtb1, Rhoj, Ripor2, Rpain, Rpgr, Rps24, Rps6ka1, Rtel1, Rtn4, Rubcn, Runxlt1, Sbf1, Scpep1, Scrn3, Sema4d, Sema6a, Serpinb6a, Serpinh1, Setd3, Sez6l, Sh3pxd2a, Shank3, Shisa6, Sidt2, Sipa1l3, Sirt1, Skil, Slain2, Slc16a5, Slc18a1, Slc22a23, Slc25a25, Slc25a40, Slc29a1, Slc31a2, Slc38a10, Slc39a13, Slc39a9, Slc4a7, Slc50a1, Smoc1 Snap23, Snrpal, Snx21, Spint2, Spire1, Ssr1, St3gal3, St3gal6, Stard13, Stoml2, Stx2, Stx4a, Stxbp5l, Sugp2, Synpo, Taf12, Tcte3, Terf1, Tex30, Thrap3, Thyn1, Tjap1, Tle3, Tlr4, Tlr7, Tmem134, Tmem209, Tmem25, Tmem44, Tpd52, Tpd52l2, Trp53i13, Trpc1, Ttc17, Ttc21b, Ttll5, Tysnd1, Ube2cbp, Ubn2, Ubxn7, Uhrflbp1, Unc13b, Uqcc1, Usp15, Usp37, Vav2, Vps13c, Wdr33, Wdr35, Wdr4, Wdr54, Wdr81, Wnk1, Yap1, Yeats2, Zbtb20, Zbtb34, Zbtb49, Zfp317, Zfp362, Zfp60, Zfp708, Zfp788, Zfp821, Zfp827, Zfp949, Zfyve28, Zgrf1, Zmat1, Zpr1, Zranb3, Flnc, Msn, Serpina3n, Cd44, Stat3, Igfbp3, Cd63, Ucp2, Sppl, Plek, Col5a3, S1pr3, Anxa2, Mt1, Sbno2, Csf1, Ch25h, Mt2, Serpinel, Vim, Col16a1, Neat1, Cd109, Dysf, C3ar1, Pros1, Tnfrsf1a, Tgm1, Socs3, Rplp0, Osmr, Cebpd, Tspan7, Srxn1, Capg, Ahnak, Fgf13, S100a10, Arpc1b, Slc6a1, Lgals3, Bcl3, Plin2, Csf2rb2, Clic1, Gfap, Aspg, Emp1, and/or Msr1, and
(ii) diagnosing the subject as having progressed from a pre-symptomatic ALS disease state to an acute ALS disease state if the value of the second Ψ score is increased or decreased relative to the first Ψ score.

12. The method of claim 11, wherein the biological sample is a blood sample, serum sample, or a tissue sample, optionally wherein the tissue sample is a CNS tissue sample or a cerebrospinal fluid (CSF) sample.

13. The method of claim 11 or 12, wherein the subject is a mammalian subject, optionally wherein the subject is a human or a mouse.

14. The method of any one of claims 11-13, wherein the detecting comprises performing RNA sequencing (RNAseq) on the biological sample.

15. The method of any one of claims 11-14, wherein the calculating comprises performing a mixture of isoforms (MISO) analysis.

16. The method of any one of claims 11-15, wherein the control sample is a biological sample obtained from a healthy subject.

17. The method of any one of claims 11-16, further comprising a step of administering a therapeutic agent to the subject.

18. The method of claim 17, wherein the therapeutic agent is a peptide, protein, nucleic acid, or small molecule.

19. The method of claim 18, wherein the peptide is a peptide vaccine that targets a RAN protein.

20. The method of claim 18, wherein the protein is an antibody, optionally wherein the antibody is an anti-RAN protein antibody.

21. The method of claim 20, wherein the anti-RAN protein antibody binds to a di-amino acid repeat region of a RAN protein.

22. The method of claim 17, wherein the therapeutic agent targets a DNA repair pathway gene or gene product.

23. A method for treating pre-symptomatic ALS in a subject, the method comprising:
administering to the subject a therapeutic agent, wherein the subject has been characterized as having pre-symptomatic ALS by the method of any one of claims 1 to 10.

24. The method of claim 23, wherein the subject is a mammal, optionally wherein the mammal is a human or a mouse.

25. The method of claim 23 or 24, wherein the subject is characterized as having a C9orf72 expansion repeat greater than 30 repeats, or wherein the subject expresses one or more RAN proteins from a C9orf72 expansion repeat.

26. The method of any one of claims 23 to 25, wherein the therapeutic agent is a peptide, protein, nucleic acid, or small molecule.

27. The method of claim 26, wherein the peptide is a peptide vaccine that targets a RAN protein.

28. The method of claim 26, wherein the protein is an antibody, optionally wherein the antibody is an anti-RAN protein antibody, optionally wherein the anti-RAN protein antibody binds to a di-amino acid repeat region of a RAN protein.

29. The method of claim 26, wherein the therapeutic agent targets a DNA repair pathway gene or gene product.

30. The method of any one of claims 1-10, further comprising a step of administering a therapeutic agent to the subject identified as having pre-symptomatic ALS.

31. The method of claim 30, wherein the therapeutic agent is a peptide, protein, nucleic acid, or small molecule.

32. The method of claim 31, wherein the peptide is a peptide vaccine that targets a RAN protein.

33. The method of claim 31, wherein the protein is an antibody, optionally wherein the antibody is an anti-RAN protein antibody, optionally wherein the anti-RAN protein antibody binds to a di-amino acid repeat region of a RAN protein.

34. The method of claim 31, wherein the therapeutic agent targets a DNA repair pathway gene or gene product.

35. The method of any one of claims 17-18, 23, 26, or 30, wherein the therapeutic agent targets one or more gene(s) identified by the method of any one of claims 1 to 10.

36. The method of any one of claims 17-18, 23, 26, or 30, wherein the therapeutic agent targets a protein or RNA which is expressed or encoded by one or more gene(s) identified by the method of any one of claims 1-10.
